(19) United States
(12) Patent Application Publication
(10) Pub. No.: US 2017/0296697 A1
(43) Pub. Date: Oct. 19, 2017

(54) USE OF REGENERATIVE CELLS IN MITIGATING BURN PROGRESSION AND IMPROVING SKIN GRAFT INCORPORATION AND HEALING

(71) Applicant: Cytori Therapeutics, Inc., San Diego, CA (US)

(72) Inventors: John K. Fraser, San Diego, CA (US); Philippe Foubert, San Diego, CA (US); Zeni Alfonso, Temecula, CA (US)

(21) Appl. No.: 15/515,870

(22) PCT Filed: Oct. 2, 2015
(86) PCT No.: PCT/US15/53856

§ 371 Date: Mar. 30, 2017

Related U.S. Application Data
(60) Provisional application No. 62/059,773, filed on Oct. 3, 2014.

(51) Int. Cl. A61L 27/24; C12N 5/077; A61L 27/38; A61L 27/56; C12N 5/0775; A61L 27/60; C12N 5/00; A61L 27/00

(52) U.S. Cl. CPC A61L 27/24; C12N 5/0667; C12N 5/0653; A61L 27/60; A61L 27/3834; A61L 27/56; A61L 27/00; C12N 5/00

(57) ABSTRACT

Described herein are compositions and methods for the mitigation of burn progression. In particular, the described herein are compositions including regenerative cells for use in preventing and reducing burn progression. Also described are compositions and methods for improving graft take and healing, and preventing and/or treating hypertrophic scars.

### STATEMENT REGARDING FEDERALLY SPONSORED R&D

The present invention was made with government support under the following contract: HHS0100201200008C awarded by the Department of Health and Human Services. The United States government has certain rights in the invention.

### BACKGROUND

Skin, or “cutis” is a bilayer organ that includes an outer, epidermal layer, and the inner, dermal layer. The epidermal layer itself comprises an outer layer of dead cells and keratin, and a basal layer of multiplying keratinocytes. The epidermal layer provides a physical barrier to toxins (e.g., bacterial, and environmental), prevents loss of moisture, and maintains body temperature. The inner, dermal layer is located between the epidermal layer and subcutaneous tissues. The dermal layer is divided into the papillary dermis, which is composed of collagen fibers, and the reticular dermis, which is composed of collagen fibers as well as cells including fibroblasts, macrophages, mast cells and adipocytes. The dermal layer also contains the microcirculation, a complex vascular plexus of arterioles, venules, and capillaries. The dermis functions to provide support for the epidermal layer, cushion the body from stress and strain, provide nutrients to and remove waste from, the epidermis and dermal layers.

Cutaneous burns are one of the most destructive insults to the skin, causing damage, scarring and even death of cutaneous (and, in some cases, subcutaneous) tissue. Burns account for over 2 million medical procedures every year in the United States. Of these, 150,000 subjects are hospitalized and as many as 10,000 subjects die (Bronzino, 1995, The Biomedical Engineering Handbook (CRC Press: Florida)).

Burns are classified depending on the lesion severity into four categories: (1) superficial or first degree (2) partial thickness or second degree burns (3) full-thickness or third degree burns, wherein the lesion involves the subcutaneous layer, and which associated with no sensitivity and white coloring; and (4) subdermal or fourth-degree burns. Partial thickness burns are further subdivided into (a) superficial partial thickness burns (b) mid partial thickness burns, and (c) deep partial thickness burns. Superficial/first-degree burns affect only the epidermis, and resolve without intervention in 3-5 days without scarring. Superficial partial-thickness burns extend through the epidermis into the papillary dermis. Superficial partial-thickness burns initially appear red and blister and are characterized by hypersensitivity and pain. Typically, superficial partial thickness burns are not associated with scarring. Deep partial-thickness burns extend into the reticular layer of the dermis. Deep partial thickness burns appear yellow or white, and may exhibit blistering. In contrast to superficial partial thickness burns, deep partial-thickness burns are associated with scarring and contracture, and often require excision and grafting. Full thickness burns extend through the entire dermal layer. Full thickness burns are characterized by scarring and contractures. Burn excision (and in some rare cases amputation), is standard in full-thickness burns. Subdermal or fourth degree burns extend through epidermal and dermal layers and into underlying fat, muscle and bone.

Primary tissue loss in burn injury arises from protein denaturation following thermal, chemical, electrical, friction, or radiation-induced burns. Post-burn, in partial and full thickness burns, necrosis occurs at the focal point of the burn source, and becomes progressively less severe at the periphery. The burn area is categorized into three zones: the zone of coagulation, the zone of stasis and the zone of hyperemia. The zone of coagulation/necrosis refers to the nonviable burn eschar nearest to the burn source. The zone of stasis surrounds the zone of coagulation, and is characterized by decreased tissue perfusion, a mixture of viable and non-viable cells, capillary vasoconstriction and ischemia. The zone of hyperemia, which surrounds the zone of stasis, comprises non-injured tissue that is characterized by increased blood flow as a compensatory reaction to the burn. Tissue in the zone of hyperemia invariably recovers. Tissue in zone of stasis is potentially salvageable, given proper intervention. If not properly treated, however, the tissue in the zone of stasis dies (e.g., as a result of necrosis and/or apoptosis), as release of inflammatory mediators, tissue edema, and/or infection further compromises blood flow to already critically injured/ischemic tissues.

The three zones of a burn are three dimensional, and loss of tissue in the zone of stasis will lead to the wound deepening as well as widening. This phenomenon is referred to as burn progression” or “burn conversion.” Hence, a burn that initially is assessed as partial thickness may progress to full-thickness with time. Both apoptosis (an active process requiring protein synthesis, i.e., energy dependent process) and necrosis (energy independent. “passive” process leading to cell death) are observed in the conversion of tissue in the zone of ischemia to non-viable tissue. See, Singer, et al (2008) Academic Emergency Medicine 15:549-554.

Tangential excision of burn wounds, escharectomy, or debridement, is regarded as the standard of care for burns that are not anticipated to heal within 3 weeks. Such burns include deep partial thickness burns and full thickness burns. Choi, et al. (2008) J Craniofac. Surg. 19:1056-60. Tissue that is already non-viable, or that is expected to become non-viable is excised in order to reduce the likelihood of infection, as non-viable, non-perfused tissue is a nidus for bacteria and fungi. Wound debridement is also widely used outside of the burn context, e.g., in cases dead, damaged, or infected tissue is present, in order to improve the healing potential of the remaining healthy tissue. As loss of the epidermal layer that normally functions to shield the individual from exposure to bacteria, fungi, and environmental toxins, the risk of infection in burn subjects is extremely high. Non-viable cells and cell debris are also a source of toxic products, thereby inciting an inflammatory response. Burn debridement has been demonstrated to reduce mortality, reduce hospital stay, and is associated with improved rates of wound healing, and reduction in subsequent scarring.

Using debridement alone, the risk of infection is still extremely high. As such, skin grafts are often used to promote healing of, and to prevent contracture and scarring of, the debrided area. Ideally, skin grafts are taken from the patient's own skin (donor sites). However, in cases where a large sized graft is needed, or where the patient is not stable, autografts may not be feasible. Furthermore, obtaining donor skin is painful, and involves risks such as infection, and destabilization of a subject whose overall health is already compromised due to the initial burn injury. In such cases, allografts (i.e., taken from other subjects of the same species), xenografts (i.e., taken from different species), and synthetic grafts are used as alternatives. Other potential complications with skin grafts include: graft failure; rejection of the skin graft; infections at donor or recipient sites; or autograft donor sites oozing fluid and blood as they heal. Certain of these complications (e.g., graft failure and rejection of the skin graft) may be somewhat mitigated by using an autograft instead of an allograft or a xenograft.

Depending upon the depth and severity of the wound/burn, the either a full thickness skin graft or a split thickness skin graft is recommended. Split-thickness skin grafts, or “STSGs” contain the epidermis and only a portion of the underlying dermis. Full thickness skin grafts contain the epidermis and the entire thickness of the dermis. Split-thickness flaps are hampered by the low degree of surgical “take.” Typically, only about 20% to 40% of the transplanted skin successfully reestablishes itself in its new position. Full-thickness flaps are even more difficult to reestablish in a new site. See, U.S. Pat. No. 4,810,693. Graft failure can arise as a consequence of one or several reasons, including inadequate excision of the wound bed, that results in non-viable tissue beneath the skin graft; inadequate vascular supply to the wound bed; hematomas and seromas forming a barrier between the bed and skin graft; shearing or displacement of the graft that prevents revascularization of the graft; and infection, which can give rise to disintegration of the graft or excessive exudate that prevents the graft from adhering to the bed. Wounds that develop secondary to radiation are less likely to support split-thickness skin grafts (STSGs) and often require adjunctive measures to optimize survival. Likewise, subjects with diabetes and other conditions that compromise the vascular system (e.g., peripheral vascular disease and the like) are also more likely to have lower skin graft “take” compared to subjects not affected by conditions that compromise the vascular system. In addition to the inherent risks associated with skin grafting, skin grafts are expensive and often are limited in supply. Accordingly, it is highly desirable to minimize (or even eliminate) tissue excision, and to minimize the amount of graft tissue used in the procedure. Burn wound progression creates a “moving target” situation in which the total body surface area (“TBSA”) of necrotic tissue requiring excision and grafting can progressively increase in the first several days after thermal trauma. On top of this, once the extent of burn requiring excision and closure is demarcated, due to the limited supply of various skin grafts, expansion of the area in which graft is required further prolongs time to complete definitive wound closure. The need for therapies that minimize burn wound progression/conversion and/or enhance skin graft incorporation and healing is evident, as reducing conversion/progression would minimize and/or prevent the need for tissue excision altogether, thereby enhancing wound closure success rates and accelerating recovery and decreasing the morbidity and mortality of burn patients. Furthermore, reducing or eliminating burn wound progression will minimize the amount of skin graft material required. Finally, the desirability of improving graft “take,” in the context of burns or other wounds which require grafting, is evident, as improved graft take improves the subject's outcome, and minimizes risks and further expenses associated with failed grafts and the need for secondary repeat graft harvest and application.

Another major concern in wound healing (e.g., healing of burn and other types of wounds) and the healing of skin grafts is the development of pathological scars, such as hypertrophic scars. Hypertrophic scarring is a cutaneous condition characterized by deposits of excessive amounts of collagen which gives rise to a raised scar. Hypertrophic scarring generally develops after thermal or traumatic injury that involves the deep layers of the dermis. When present over joints, hypertrophic scarring can cause severe joint contracture and eventually lead to erosion of the underlying bone, secondary to disuse. See, Aarabi, et al. PLOS Medicine (2007) 4(9):1464-1470. Efforts to limit scar formation, e.g., in burn patients have relied largely on immediate skin replacement with human split-thickness autografts or allografts or with synthetic dermal analogs such as Integra™. Even with skin grafting, however, clinicians recognize that hypertrophic scarring remains a terrible clinical problem. See, e.g., Sheridan, et al. (2004) J Am. Col. Surg. 198:243-263.

Clinical experience suggests that hypertrophic scarring is an aberrant form of the normal processes of wound healing. Singer, et al. (1999) N Engl J Med. 341:738-746. However, the etiology of the overexuberant fibrosis is unknown. The pathophysiology of hypertrophic scar formation involves a constitutively active proliferative phase of wound healing and disordered production of collagen (for example, excessive production and disorganized orientation of collagen). Scar tissue has a unique structural makeup that is highly vascular, with inflammatory cells and fibroblasts contributing to an abundant and disorganized matrix structure. Although the pathogenesis is not well understood, high expression of TIMP-1 and inhibition of MMP-1 activity have been implicated in causing a decrease in the degradation of collagen during wound repair, and are thought to contribute to the formation of hypertrophic scars. The net result is that the original skin defect is replaced by a dysfunctional mass of tissue. For example, while the scar may maintain, to a sufficient extent, the barrier function of normal skin, it does not maintain the flexibility and softness required to permit normal motion of the underlying and adjacent structures. This can lead to sequelae such as debilitating limited range of motion of a joint and to facial immobility and associated inability to achieve facial expressions. The ratio of type III collagen to type I collagen has also been reported to be altered/elevated in hypertrophic scars when compared to non-pathological scars. Oliviera, et al. (2009) Int. Wound J. 6(6):445-452. Another hallmark of hypertrophic scars is elevated levels of a-smooth muscle actin (α-SMA). In contrast to non-pathological scars and keloid scars, hypertrophic scars have characteristic prominent, vertical vessels present in the scar tissue. Given the adverse consequences resulting from hypertrophic scarring—including loss of function, restriction of movement, disfigurement and the like, preventative and therapeutic options are desirable.

### SUMMARY

Disclosed herein are compositions and methods useful for the treatment of wounds. In one aspect, the embodiments disclosed herein relate to the treatment of burns. Accordingly, some embodiments relate to compositions and methods for preventing or mitigating wound progression. In such embodiments, a subject having a burn, and at risk of developing burn progression can be identified. A therapeutically effective amount of a composition comprising regenerative cells sufficient to mitigate progression of the burn can be administered to the subject. The methods can also include the step of debriding or performing an escharectomy on the burn, and/or measuring or calculating burn progression.

In a second aspect, the embodiments disclosed herein relate to compositions and methods for enhancing incorporation of a skin graft into a recipient wound site. Such embodiments can include the steps of providing a skin graft, administering to the skin graft a composition comprising regenerative cells to create a fortified skin graft; and applying the fortified skin graft to the recipient wound site. In an alternative embodiment, the methods can include the steps of providing a skin graft, administering a composition comprising regenerative cells systemically to the subject and/or locally to the wound site. The skin graft can be applied to the recipient wound site either before or after application of the regenerative cells.

A third aspect of the embodiments disclosed herein relate to compositions and methods for preventing or minimizing the formation of hypertrophic scar in a deep partial thickness or full thickness wound. Such embodiments can include the steps of identifying a subject having a deep partial thickness or full thickness wound, and administering to the subject, e.g., systemically or locally to the deep partial thickness or full thickness wound, a composition comprising regenerative cells.

In a fourth aspect, the embodiments disclosed herein provide compositions and methods of reducing or eliminating a hypertrophic scar. The methods can include the step of identifying a subject having a hypertrophic scar; and administering a composition comprising regenerative cells to the subject, e.g., systemically and/or locally to the hypertrophic scar. The methods can include the further steps of debriding the scar tissue prior to administration of the composition comprising regenerative cells.

In a fifth aspect, the embodiments disclosed herein relate to compositions and methods of treating contracture in a subject in need thereof. A subject with a joint or muscle contracture can be identified, and a composition comprising regenerative cells can be administered to the subject, thereby treating the contracture. The methods can include the steps of assessing range of motion, scarring, and the like.

### BRIEF DESCRIPTION OF THE DRAWINGS

FIG. 1 is a chart showing the experimental process flow for the combined radiation and thermal injury experiments described in Example 1, below.

FIG. 2 is an illustration depicting local injection sites into burn tissue, as performed in the experiments in Example 1, below.

FIG. 3 is an image showing the areas of (a) contraction (the total area not covered by unwounded skin); and (b) epithelialization (the area within the wound showing evidence of neo-epithelialization), of an exemplary burn wound analyzed in Example 1. The thick, solid line indicates the area of biopsy. The inner dotted line indicates the boundary of re-epithelialization. The outer dotted line shows the wound boundary for assessment of contraction.

FIG. 4 is an illustration outlining the scheduling and processing of burn wounds (immunohistochemistry [IHC] or snap-freezing for molecular analysis) for wound biopsy (2 or 4 biopsy collection configuration), as performed in the experiments in Example 1, below. FIGS. 5A-5C are graphs showing measurement of hematology parameters over time in control animals (Gr1a), animals receiving locally administered adipose-derived regenerative cells (Gr1b), and animals receiving intravenously administered adipose-derived regenerative cells (Gr1c), as described in Example 1. FIG. 5A shows absolute white blood cell count. FIG. 5B shows absolute neutrophil count. FIG. 5C shows absolute platelet count. FIG. 5D shows absolute lymphocyte count.

FIGS. 6A-6B show phase contrast photomicrographs (magnification 100×) of porcine adipose-derived regenerative cells plated under angiogenic conditions as described in Example 1. Micrograph of cells from study animal #5341010 (FIG. 6A). Micrograph of cells from animal #5344302 (FIG. 6B). The arrows point to tube-like structures.

FIGS. 7A and 7B are representative phase contrast micrographs (magnification 100×) showing cells from animal #5341010 prior to (FIG. 7A) and after (FIG. 7B) oil red O staining, in the adipogenesis assay described in Example 1.

FIG. 8 is a graph showing percent wound contraction at various time points for animals in Group 1a (control) Group 1b (locally delivered adipose-derived regenerative cells) and Group 1C (intravenously delivered adipose-derived regenerative cells) as described in Example 1.

FIGS. 9A-9D are bar graphs showing the percent re-epithelialization 7 days post-injury (FIG. 9A); the percent epithelial coverage 7 days post-injury (FIG. 9B); the activated epithelium area (μm²) (FIG. 9C); and the percent proliferating epithelium (FIG. 9D) in animals in Group 1a (LR control), Group 1b (local adipose-derived regenerative cell delivery), and Group 1c (intravenous adipose-derived regenerative cell delivery), as described in Example 1.

FIG. 10A is a photograph of masson trichrome staining on biopsy of a sample of eschar as described in Example 2. FIGS. 10B and 10C are details of FIG. 10A. The black arrows indicate hemorrhage in adipose tissue.

FIG. 11 Is a photograph (magnification 200×) of Oil Red O staining on adipose-derived regenerative cells from eschar tissue subjected to the adipogenesis assay as described in Example 2.

FIGS. 12A-12C are photographs of immunostained vessel-like structures formed in angiogenic cultures of adipose derived regenerative cells isolated from an exemplary eschar sample as described in Example 2.

FIG. 13 is a chart showing the experimental process flow for the combined radiation and thermal injury experiments described in Example 3, below.

FIG. 14 is a graph showing the percentage of open wound area of individual wounds at day 14 post-burn induction in Group D control (LR) and test (ADRC)-treated wounds, as described in Example 3, below.

FIG. 15 is a scatter plot showing the percentage of wound epithelialization for all wounds in LR and ADRC-treated animals in Group D at study day 14. N=16 wounds per control and test cohorts, as described in Example 3, below.

FIGS. 16A-16D: FIGS. 16A and D: representative images showing neovascularization of deep granulation tissue at day 14 and 21, respectively, in animals in Group D receiving vehicle alone. FIGS. 16B and 16D are representative images showing neovascularization of deep granulation tissue at day 14 and 21, respectively, in animals in Group D receiving ADRCs. Wound biopsies collected from animals receiving vehicle alone or local ADRCs were stained with CD31 (endothelial marker). Arrows show CD31-positive blood vessels. Scale bar=300 μm. Bottom panel: quantification of microvessel density at day 14 and 21 in LR- and ADRCs-treated animals. n=4 animals per group; 6 wounds total each treatment condition, as described in Example 3, below.

FIG. 17 is a graph showing epithelial thickness in LR- and ADRCs-treated wounds of Group D animals, as described in Example 3, below.

FIGS. 18A-B Histological Assessment of Granulation Tissue Maturation. FIG. 18A depicts the scale used for biopsy histology, used in the experiments described in Example 3, below. FIG. 18B is a graph of tissue organization over time in wounds treated with INTEGRA® or INTEGRA® supplemented with ADRC. FIG. 18C is a graph showing granulation tissue thickness over time in wounds treated with INTEGRA® or INTEGRA® supplemented with ADRC. Mean granulation tissue thickness was greater ADRC+Integra treated wounds by Day 21 than Integra controls.

FIGS. 19A and 19B are graphs showing the microvessel density at days 14 and 21 in wounds treated with INTEGRA® or INTEGRA® supplemented with ADRC. FIGS. 19C and 19D are graphs showing the total CD312 stain at days 14 and 21 in wounds treated with INTEGRA® or INTEGRA® supplemented with ADRC. FIGS. 19E and 19F are graphs showing the total lumen area at days 14 and 21 in wounds treated with INTEGRA® or INTEGRA® supplemented with ADRC.

FIGS. 20A and 20B are graphs showing the percent of INTEGRA® matrix filled and the number of cells per mm² in wounds treated with INTEGRA® or INTEGRA® supplemented with ADRC. FIG. 20C is a graph showing the number of vessels/mm² in wounds treated with INTEGRA® or INTEGRA® supplemented with ADRC.

FIG. 21 is a graph showing epithelial coverage on biopsies collected at day 21 in Group C, as described in Example 3 below (n=4 animals per group; 6 wounds/group).

FIG. 22 is a graph showing quantification of Microvessel Density (MVD) at day 7, 14 and 21 in animals receiving TISSEEL®+vehicle or TISSEEL®+ADRCs within superficial granulation tissue, as described in Example 3, below (n=4 animals per group; 6 wounds/group).

FIG. 23 scattergram from sample #E5 showing the scatter distribution of cells regarding CD34 vs CD90 staining as described in Example 2.

### DETAILED DESCRIPTION

The embodiments disclosed herein are based, in part, upon the discovery that compositions that include regenerative cells can function to mitigate, reduce and prevent burn progression/conversion, and/or secondary injury and scarring arising from burn. The embodiments also are based, in part, upon the finding that regenerative cells could be readily obtained from adipose tissue from subjects suffering from thermal burn injury, including the adipose from eschar tissue. The embodiments are further based, in part, upon the finding that regenerative cells could be readily obtained from adipose tissue from subjects suffering from radiation injury. Finally, the embodiments disclosed herein are also based, in part, upon the discovery that compositions that include regenerative cells are useful in preventing and/or treating pathological scarring, e.g., hypertrophic scarring following a deep-partial thickness or full thickness wound (such as a burn or the like).

Definitions

As used herein, the term “about,” when referring to a stated numeric value, indicates a value within plus or minus 10% of the stated numeric value.

As used herein, the term “derived” means isolated from or otherwise purified or separated from. For example, adipose-derived stern and other regenerative cells are isolated from adipose tissue. Similarly, the term “derived” does not encompass cells that are extensively cultured (e.g., placed in culture conditions in which the majority of dividing cells undergo 3, 4, 5 or less, cell doublings), from cells isolated directly from a tissue, e.g., adipose tissue, or cells cultured or expanded from primary isolates. Accordingly, “adipose derived cells,” including adipose-derived stem and other regenerative cells and combinations thereof, refers to cells obtained from adipose tissue, wherein the cells are not extensively cultured, e.g., are in their “native” form as separated from the adipose tissue matrix.

As used herein, a cell is “positive” for a particular marker when that marker is detectable. For example, an adipose derived regenerative cell is positive for, e.g., CD73 because CD73 is detectable on an adipose derived stem or regenerative cell in an amount detectably greater than background (in comparison to, e.g., an isotype control or an experimental negative control for any given assay). A cell is also positive for a marker when that marker can be used to distinguish the cell from at least one other cell type, or can be used to select or isolate the cell when present or expressed by the cell.

As used herein, “regenerative cells” refers to any heterogeneous or homogeneous population of cells obtained using the systems and methods of embodiments disclosed herein which cause or contribute to complete or partial regeneration, restoration, or substitution of structure or function of an organ, tissue, or physiologic unit or system to thereby provide a therapeutic, structural or cosmetic benefit. Examples of regenerative cells include: adult stern cells, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes.

Accordingly, adipose-derived regenerative cells (“ADRCs”) as used herein refers to any heterogeneous or homogeneous cell population that contains one or more types of adipose-derived regenerative cells including adipose-derived stem cells, endothelial cells (including blood and lymphatic endothelial cells), endothelial precursor cells, endothelial progenitor cells, macrophages, fibroblasts, pericytes, smooth muscle cells, preadipocytes, kertainocytes, unipotent and multipotent progenitor and precursor cells (and their progeny), and lymphocytes. Adipose-derived stern cells comprise at least 0.1% of the cellular component of adipose-derived regenerative cells.

Similarly, “bone marrow-derived regenerative cells” (“BMRCs”) refers to any heterogeneous or homogeneous cell population that contains one or more types of bone marrow-derived regenerative cells including bone marrow-derived stem cells, endothelial cells (including blood and lymphatic endothelial cells), endothelial precursor cells, endothelial progenitor cells, macrophages, fibroblasts, pericytes, smooth muscle cells, preadipocytes, keratinocytes, unipotent and multipotent progenitor and precursor cells (and their progeny), and lymphocytes.

In some contexts, the term “progenitor cell” refers to a cell that is unipotent, bipotent, or multipotent with the ability to differentiate into one or more cell types, which perform one or more specific functions and which have limited or no ability to self-renew. Some of the progenitor cells disclosed herein may be pluripotent.

As used herein the phrase “adherent cells” refers to a homogeneous or heterogeneous population of cells which are anchorage dependent, i.e., require attachment to a surface in order to grow in vitro.

In some contexts, the term “adipose tissue-derived cells” refers to cells extracted from adipose tissue that has been processed to separate the active cellular component (e.g., the cellular component that does not include adipocytes and/or red blood cells) from the mature adipocytes and connective tissue. Separation may be partial or full. That is, the “adipose tissue-derived cells” may or may not contain some adipocytes and connective tissue and may or may not contain some cells that are present in aggregates or partially disaggregated form (for example, a fragment of blood or lymphatic vessel comprising two or more cells that are connected by extracellular matrix). This fraction is referred to herein as “adipose tissue-derived cells,” “adipose derived cells,” “adipose derived regenerative cells” or “ADC.” Typically, ADC refers to the pellet of cells obtained by washing and separating the cells from the adipose tissue. The pellet is typically obtained by concentrating a suspension of cells released from the connective tissue and adipose tissue matrix. By way of example, the pellet can be obtained by centrifuging a suspension of adipose-derived cells so that the cells aggregate at the bottom of a centrifuge container, e.g., the stromal vascular fraction. In some embodiments, the adipose-derived cell populations described herein include, among other cell types, leukocytes. In some embodiments, the adipose-derived cell populations described herein include, among other regenerative cell types, endothelial cells.

In some contexts, the term “adipose tissue” refers to a tissue containing multiple cell types including adipocytes and vascular cells. Adipose tissue includes multiple regenerative cell types, including adult stem cells (ASCs), endothelial progenitor and precursor cells, pericytes and the like. Accordingly, adipose tissue refers to fat, including the connective tissue that stores the fat.

In some contexts, the term “unit of adipose tissue” refers to a discrete or measurable amount of adipose tissue. A unit of adipose tissue may be measured by determining the weight and/or volume of the unit. In reference to the disclosure herein, a unit of adipose tissue may refer to the entire amount of adipose tissue removed from a subject, or an amount that is less than the entire amount of adipose tissue removed from a subject. Thus, a unit of adipose tissue may be combined with another unit of adipose tissue to form a unit of adipose tissue that has a weight or volume that is the sum of the individual units.

In some contexts, the term “portion” refers to an amount of a material that is less than a whole. A minor portion refers to an amount that is less than 50%, and a major portion refers to an amount greater than 50%. Thus, a unit of adipose tissue that is less than the entire amount of adipose tissue removed from a subject is a portion of the removed adipose tissue.

As used herein, the term “ROS” and “RNS” refer to reactive oxygen species and reactive nitrogen species, respectively. ROS and RNS include compounds such as hydrogen peroxide, peroxynitrate, hydroxyl radical (.OH), nitrogen dioxide radical (.NO₂) and carbonate radical (.CO₃). As used herein, the term “lipid peroxidation,” or “lipid peroxidation products” or “LPPs” can include, but are not limited to malondialdehyde (MDA) and 4-hydroxynonenal (HNE), acrolein, and the like.

As used herein, the term “skin substitute” or “skin graft” refers to anything that substitutes for any of the skin functions provided by the native skin at that site prior to injury or development of a wound. Skin substitutes or skin grafts can be allografts (e.g., cadaveric grafts, or the like), or xenografts. Skin grafts can also be autografts (ie: grafts obtained from the patient receiving the graft). In certain embodiments, the graft can be in a dispersed form (e.g., a skin graft that has been meshed or treated enzymatically to create a completely or partially dispersed suspension of skin cells including keratinocytes that is then applied to the area in need of coverage). In certain embodiments, the graft can comprise cultured cells (e.g., cultured keratinocytes and/or dermal cells with or without a supportive scaffold). Preferably, a skin substitute should in some way be incorporated into the healing wound. Cultured or artificial dressings, therefore, may be used as a substitute for the epidermal layer, the dermal layer, or both layers simultaneously. Some grafts are used to provide skin function for a limited period (temporary coverage). For example, allografts and xenografts are usually removed prior to definitive wound treatment or skin grafting.

The compositions and embodiments disclosed herein are useful for treating subjects with burn injury, and/or in subjects in need of a skin graft (e.g., skin graft, skin substitute, or the like). Accordingly, the term “subject” can refer to any mammal including, but not limited to mice, rats, rabbits, guinea pigs, pigs, dogs, cats, sheep, goats, cows, horses, primates, such as monkeys, chimpanzees, and apes, and humans. In some embodiments, the subject is a human. The term “subject” can be used interchangeably with the terms “individual” and “patient” herein. As explained in further detail below, in some embodiments, the subject has radiation injury (e.g., acute radiation injury), and a deep partial thickness or full thickness wound, such as a burn.

Burn Progression

Burn wounds continue to mature for several days following initial insult, confounding burn classification and treatment protocols. Damage to the skin continues several days post-insult, as tissue in the zone of stasis undergoes necrosis and/or apoptosis. Both apoptosis and necrosis occurs in the ischemic zone of burns. Apoptotic dermal cells are found at a much higher frequency in deep partial-thickness burns compared to superficial partial thickness burns, and persist over 20 days. See, e.g., Gravante, et al. (2006) Surgery 139:854-855.

Burn progression involves a complex concert of events, which include oxidative stress, persistent inflammation, and compromised perfusion. See, e.g., Shupp, et al. (2010) J. Burn Care & Res. 31:849-873. As discussed in further detail below, the methods and compositions disclosed herein function to ameliorate one or more of these pathways, thereby minimizing and/or preventing burn progression. As such, the methods and compositions disclosed herein can advantageously reduce or minimize the area of the recipient site of a skin graft, in some instances, eliminate the need for a skin graft altogether following burn.

Oxidative stress transpires as a result of an imbalance between the systemic generation of reactive oxygen species and a biological system's ability to readily detoxify the reactive intermediates and/or to repair the resulting damage. Various different pathways converge to create oxidative stress and an over-abundance of free radicals in burn. First, thermal burns can directly generate free radicals by hemolytic bond fission caused by heat. Burn also causes an increased activity of xanthine oxidase and NADPH oxidase, as well as increased nitric oxide (“NO”) production, e.g., in proliferating keratinocytes, capillary endothelial cells and arterial smooth muscle cells. See, e.g., Shupp, et al. (2010) J. Burn Care & Res. 31:849-873. Xanthine oxidase and NADPH oxidase generate the damaging ROS hydrogen peroxide and superoxide. NO in turn interacts with superoxide radicals to produce the highly reactive peroxynitrite compound, a reactive nitrogen species. The increase in reactive oxygen species (“ROS”) and reactive nitrogen species (“RNS”) is compounded by reductions in oxidative defenses, including reductions in superoxide dismutase (“SOD”), glutathione, ascorbic acid, and α-tocopherol associated with burn.

Excessive ROS and RNS cause multiple deleterious effects, including cellular damage, e.g., to DNA, proteins, lipids (generating lipid peroxidation products, or “LPPs”), and other structural cellular components, and can ultimately lead to apoptosis, thereby causing and/or worsening burn progression. As such, ROS and RNS are key players in burn progression. LLPs have also been shown to play a role in macroscopic interspace necrosis, neutrophil infiltration, and thrombosis, thereby promoting burn progression. See, e.g., Taira, et al. (2009) J. Burn Care Res. 30:499-504.

In concert with the cellular damage, oxidative stress exacerbates and contributes to persistent inflammation, which is also implicated in burn progression. ROS induce the expression of pro-inflammatory cytokines through the action of NF-kB. For example, damage to cell membranes (e.g., arising from apoptosis or necrosis due to the initial burn insult and/or consequent ROS and/or RNS damage), results in a dynamic cascade of inflammatory mediators. Prolonged or persistent inflammation in turn results in collagen degradation and keratinocyte apoptosis, thereby furthering burn progression.

In addition to the pro-inflammatory effects arising from oxidative stress and damage, devitalized tissue, e.g., arising from an initial burn insult, is also pro-inflammatory. Devitalized tissue has exposed C3b binding sites as well as self-antigens, and serves as a powerful activator of the alternate complement system. In addition, bacteria that colonize the necrotic tissues are also powerful activators of the complement system. Activation of the complement cascade is known to be involved in burn wound progression. See, e.g., Henze, et al. (1997) Burns 23:473-477. Activation of the complement cascade leads to the diffusion of chemotactic factors in the surrounding blood stream. Complement split factors, in turn, activate neutrophils, leading to regional endothelial cell adhesion and migration. At the same time, lymphokines originally stored in the tissues or subsequently produced by invading cells are released in the wound itself. This stimulates monocyte invasion and potentiates their maturation into tissue macrophages, which are the central cells responsible for wound clearing of devitalized tissues, bacteria, and large amounts of self-antigens by the process of phagocytosis. This process is further enhanced by the opsonizing properties of the complement factors. Oxygen free radicals, lysosomes, and inflammatory cytokines are all elevated as a result of phagocytosis. Complement activation and intravascular stimulation of neutrophils result in the production of cytotoxic free radicals.

Cellular release of pro-inflammatory cytokines such as TNFα, IL-1, IL-6, IL-8, and IL-10 occurs following burn injury. See, e.g., Dorst, et al. (1993) J. Trauma 35(3): 335-339; Molloy, et al. (1993) J. Immunol. 151: 2142-2149. Abnormal levels of proinflammatory mediators, such as tumor necrosis factor alpha (TNFα), interleukin-1b (IL-1b), interleukin-6 (IL-6), interleukin-8 (IL-8), and interleukin-10 (IL-10), have been reported both systemically and locally in burn patients. Necrotic expansion in burn progression/ conversion is driven by a microenvironment characterized by elevated levels of pro-inflammatory mediators and reduction of pro-inflammatory cytokines. Blocking of pro-inflammatory molecules has been demonstrated to advantageously reduce or mitigate burn progression. Sun, et al. (2012) Wound Repair Regen. 20(4):563-72. Leukocyte infiltration is also involved in burn progression. Blocking neutrophil adhesion to the endothelium, e.g., via systemic administration of blocking antibodies, has been demonstrated to reduce wound conversion in an animal model. Choi, et al. (1995) Plastic Reconst. Surg. 96(5): 1007-1250. The embodiments disclosed herein are based, in part, on the discovery that the regenerative cells disclosed herein can advantageously function to alter the microenvironment of partial and full thickness burns, thereby preventing and/or minimizing necrotic and/or apoptotic expansion.

The compositions disclosed herein can advantageously function to stop or inhibit the expansion of the zone of coagulation or necrotic tissue of a burn, or to minimize the expansion of the zone of coagulation or necrotic tissue of a burn. Accordingly, in some embodiments, provided herein are methods for minimizing and/or preventing wound progression in a subject in need thereof. The methods can include administering a composition comprising regenerative cells to a subject at risk of burn progression, e.g., a subject having a deep partial thickness wound or a full thickness wound, such as a burn, or the like. Accordingly, in some embodiments, the methods disclosed herein eliminate the need for skin grafting. Without being limited by a particular theory, the regenerative cells disclosed herein (e.g., mesenchymal stromal cells) can prevent burn progression by one or several mechanisms, including, but not limited to minimizing or reducing oxidative stress and/or damage following burn injury, modulating the inflammatory response following burn injury (e.g., by dampening or reducing proinflammatory cytokines), modulating leukocyte infiltration into the zone of stasis, and enhancing, increasing, or restoring bloodflow in the zone of stasis.

Accordingly, provided herein are methods to reduce or minimize oxidative stress and/or damage following burn injury e.g., in the zone of stasis, in a subject in need thereof, that includes administration of a composition that includes regenerative cells as described herein. Other methods relate to the modulation of inflammation following burn injury (e.g., dampening or reducing the local concentration of inflammatory cytokines in the zone of stasis, dampening or reducing the infiltration and/or extravasation of inflammatory leukocytes in the zone of stasis, modulating polarization of immune cells to an anti-inflammatory phenotype, and the like), in a subject in need thereof, that includes administering regenerative cells as described herein. Provided herein are methods of increasing or enhancing blood flow, e.g., in the zone of stasis, following burn injury, that includes administering regenerative cells as described herein to the subject.

Methods of Mitigating Burn Progression/Conversion

In some embodiments provided are methods for reducing burn progression in subjects in need thereof In certain embodiments, the subject may be a mammal, e.g., preferably a mouse, rat, rabbit, pig, minipig, dog, cat, horse, monkey ape, human, or the like. In some embodiments, the subject may have concomitant radiation injury. Some embodiments provide methods for reducing or preventing burn progression in a subject with radiation injury that has a deep partial thickness or full thickness burn injury. In some embodiments, the radiation injury is acute radiation injury. In some embodiments, the burn injury covers more than 5%, more than 10%, more than 15%, more than 20%, more than 25%, more than 30%, or more, of the total body surface area of the subject.

In some embodiments, the methods described herein can completely prevent burn progression. That is, the zone of coagulation of the burn does not expand past its initial area following the burn injury. In some embodiments, the zone of coagulation of the burn does not expand past its area prior to treatment with a composition as disclosed herein. In some embodiments, the zone of coagulation does not expand more than 5%, 10%, 15%, 20%, 25%, 30%, 35%, 40%, 50%, or more, following treatment with a composition as disclosed herein. In some embodiments, the zone of stasis remains unchanged following administration of a composition as disclosed herein. In some embodiments, the zone of stasis exhibits less than 5%, less than 10%, less than 15%, less than 20%, less than 25%, less than 30%, less than 35%, less than 40%, less than 45%, less than 50%, or so, conversion to devitalized tissue. Accordingly, in some embodiments, administration of the compositions disclosed herein can preserve 100%, 95%, 90%, 85%, 80%, 75%, 70%, 65%, 60%, 55%, 50%, or so, of tissue in the zone of stasis of the burn.

In some embodiments, mitigating or reducing burn progression, or “treating” a patient as disclosed herein, can reduce the amount of tissue necrosis and/or apoptosis compared to the amount of tissue necrosis expected in the absence of regenerative cell administration. For example, where a patient has received a thermal burn, the administered regenerative cells can reduce the progression of burn injury in the zone of ischemia and inhibit the conversion of partial thickness injuries into full thickness necrosis. In some embodiments, the methods disclosed herein can eliminate burn progression or conversion.

The various zones of partial and full thickness burns (i.e., the zone of coagulation, the zone of stasis and the zone of hyperemia) were first described in 1953. Jackson, et al. (1953) Br. J. Surg. 40:588. Accordingly, identification of the zone of coagulation, the zone of stasis and the zone of hyperemia of a burn are widely known. Non-limiting examples of methods useful for identifying the different zones of burns include, but are not limited to, those described in US Patent Application Publication No. 2007/0197895, U.S. Pat. No. 8,435,750, and International Patent Application No's WO 2013/110021 and WO 2007/130,423A2, and the like.

In some embodiments, administration of the compositions as disclosed herein prevent or minimize conversion of a superficial partial-thickness burn to a mid partial thickness burn, a deep partial thickness burn, a full thickness burn, or a fourth-degree burn. Superficial second-degree burns involve the entire epidermis to the basement membrane and no more than the upper third of the dermis. Mid-dermal burns involve destruction of the epidermis through the middle third of the dermis. Deep second-degree burns involve the entire epidermis, and at least two thirds of the dermis. Fourth-degree burns extend through the epidermal and dermal layers of the skin, and into underlying tissue (e.g., muscle, tendon, ligament, bone, or the like). In some embodiments, administration of the compositions as disclosed herein prevent or minimize conversion or progression of (or the amount of tissue converted) a mid partial-thickness burn to a deep partial thickness burn, a full thickness burn, or a fourth-degree burn. In some embodiments, administration of the compositions as disclosed herein prevent or minimize conversion or progression of a deep partial thickness burn to a full thickness burn or a fourth-degree burn. In some embodiments, the compositions disclosed herein prevent or minimize the conversion or progression of a full thickness wound to a fourth degree burn.

In some embodiments, a subject at risk of burn conversion or burn progression is identified, e.g., self-identified, or identified by another person. Accordingly, in some embodiments, an individual with a second-degree, or partial thickness burn is identified. It is recognized that many patients will exhibit heterogeneous burn depth with certain areas of the injury constituting, for example, full thickness injury and other areas constituting deep partial and/or partial thickness wounds, and/or fourth degree wounds. In some embodiments, the subject has a superficial second-degree burn. In some embodiments, the subject has a mid second-degree burn, or mid-dermal burn. Mid-dermal wounds exhibit larger zones of stasis than superficial second-degree burns. Subjects with mid-dermal burns are at high risk of burn progression/burn conversion. In some embodiments, the subject has a deep second-degree, or deep dermal burn. In some embodiments, the subject has a full-thickness or third-degree burn, extending through the entire dermal layer. In some embodiments, the subject has a fourth-degree, or sub-dermal burn. In some embodiments, the subject has radiation injury, e.g., cutaneous or acute radiation injury. For example, in some embodiments, the subject at risk of burn progression has been exposed to 2 gray or more. In some embodiments, the subject has radiation injury and has suffered from a thermal, electrical or chemical burn.

The skilled artisan will appreciate that any art-accepted technique to classify burn depth is useful in the embodiments disclosed herein. For example, in some embodiments, burn depth is assessed visually. In some embodiments, burn depth is classified by one or more biopsies followed by histological examination. See, e.g. Chvapil et al, 1984, Plast. Reconstr. Surg. 73:438-441. Other methods of classifying burn depth useful in the embodiments disclosed herein include, but are not limited to, those described in U.S. Pat. Nos. 7,860,554, 5,701,902; 4,170,987, Canadian Patent Application 2,287,687, Mason et al. (1981), Burns 7:197-202, Park et al. (1998) Plast. Reconstr. Surg. 101:1516-1523, Brink et al. (1986) Invest. Radiol. 21:645-651, and Afromowitz et al. (1987) IEEE Trans Biomed Eng BME34:114-127, each of which is herein incorporated by reference.

Once identified, the subject can be administered a composition comprising regenerative cells according to the disclosure herein. In some embodiments, wound progression or conversion can be analyzed or measured prior to and/or following administration of regenerative cells as disclosed herein. For example, in some embodiments, the viability of tissue in the zone of stasis can be measured. The skilled artisan will readily appreciate that any art-accepted methods of determining tissue viability—either known or discovered in the future—are useful in the embodiments disclosed herein. For example, the area of devitalized tissue can be assessed visually, histologically (using biopsies, for example), or using other methods, including but not limited to those described in International Patent Application Publication No. WO 2001/078587, WO 2001/054580, WO 2005/002425A2, WO 1991/012766A, U.S. Pat. No. 8,221,989, and the like.

In some embodiments, the level of oxidative stress or oxidative damage or lipid peroxidation can be measured prior to and/or following administration of regenerative cells as described herein. Oxidative damage and lipid peroxidation can be measured using art-recognized methods or methods discovered in the future. By way of example, the methods described in Bosken, et al., “Assessments of Oxidative Damage and Lipid Peroxidation After Traumatic Brain Injury and Spinal Cord Injury” in Animal Models of Acute Neurological Injuries II, Chen, et al. Ed., (c) 2012, Humana Press, New York, N.Y., pp. 347-375; Pratico, et al. (2002) J. Neuro. 80(5): 894-898 can be used to measure lipid peroxidation.

In some embodiments, the level of bloodflow in the zone of ischemia can be measured prior to and/or following administration of regenerative cells as described herein. In some embodiments, modulation of an immune response (e.g., either local or systemic), can be measured, e.g., using any method now known or discovered in the future, prior to and/or following administration of the regenerative cells as described herein. Accordingly, in some embodiments, the level of bloodflow is assessed using Laser Doppler imaging, or any other technique known or discovered in the future. In some embodiments, the methods include analysis of vascular structures, e.g., in the zone of ischemia. For example, in some embodiments, the amount or number of CD31-positive structures can be determined.

In some embodiments, modulation of the immune response can be measured prior to and/or following administration of regenerative cells as described herein. For example, in some embodiments, the level of proinflammatory modulators (e.g., TNFα, IFNγ, IL-1, IL-2, IL-3, IL-6, IL-12, IL-18, and the like) can be determined (e.g., in tissue samples, in whole blood, in plasma, or the like) using any art-accepted method, or any method discovered in the future. In some embodiments, number and/or types of leukocytes in the zone of stasis can be measured or analyzed prior to and/or following administration of regenerative cells as described herein. The numbers of infiltrating macrophages and T cells within the burned area can be readily determined, e.g., by analysis using anti-F4/80 and anti-CD3 antibodies, respectively. In some embodiments, the ratio of different immune cells can be measured prior to and/or following administration of regenerative cells as described herein. By way of example only, in some embodiments, the methods include the step of determining the ratio of M2:M1 macrophages prior to and/or following administration of regenerative cells as described herein. The ratio of M2:M1 cells can be readily determined using art-accepted means, including for example, measuring the ratio of CD206/CD11cell surface markers (e.g., in the blood) as described in Fujisaka (2009) Diabetes 58(11): 2574-2582.

Methods of Improving Skin Grafting and Skin Graft Healing

Also provided herein are methods for improving skin grafting, incorporation of a graft into underlying tissue, or “take” of a skin graft. The skilled person will readily appreciate that the embodiments disclosed herein are useful in the treatment of a variety of types of wounds involving the placement of a graft to aid in the healing of the wound, e.g., in instances where the area of skin loss is too big to be closed using local skin and stitches alone. For example, the embodiments disclosed herein are useful in the treatment of burns, e.g., including those in which burned tissue is excised. Other exemplary types of wounds in which the methods and compositions disclosed herein are used include, but are not limited to non-healing wounds, e.g., including chronic wounds and ulcers (for example pressure wounds, wounds and ulcers associated with diabetes, peripheral vascular disease, trauma, and the like), various traumatic wounds, e.g., caused by mechanical, chemical, insect or other animal sources, and the like. For example, the methods described herein are useful in incorporation of grafts following surgical removal of cancerous, devitalized, or infected tissue and following injury from exposure to chemical agents including chemical warfare agents (e.g., vesicants and alkylating agents) where exposure could occur in the course of industrial accident, warfare, terrorist attack, or other means.

The ultimate success of a skin graft, or its “take,” depends on nutrient uptake and vascular ingrowth from the recipient bed, which occurs in 3 phases. The first phase takes place during the first 24-48 hours. The graft is initially bound to the recipient site through formation of a fibrin layer and undergoes diffusion of nutrients by capillary action from the recipient bed by a process called plasmatic imbibition. The second phase involves the process of inosculation, in which the donor and recipient end capillaries are aligned and establish a vascular network. Revascularization of the graft is accomplished through those capillaries as well as by ingrowth of new vessels through neovascularization in the third and final phase, which is generally complete within 4-7 days. Reinnervation of skin grafts begins approximately 2-4 weeks after grafting and occurs by ingrowth of nerve fibers from the recipient bed and surrounding tissue. Sensory return is greater in full-thickness grafts because they contain a higher content of neurilemmal sheaths. Similarly, hair follicles may be transferred with a graft, which allows the graft to demonstrate the hair growth of the donor site.

In some embodiments, the methods disclosed herein relate to improving the incorporation of a graft into the underlying tissue of a wound, such as a burn (e.g., following escharectomy, or the like), a chronic non-healing wound, or the like. Some embodiments relate to reducing the time between wound debridement and application of a skin graft, skin substitute or other scaffold. Regenerative cells as described herein can be administered to a debrided wound bed to create a fortified wound bed, and a skin graft can subsequently be applied to the fortified wound bed. By way of example only, a composition comprising regenerative cells as described herein can be injected into (e.g., at one or more sites) the debrided wound bed. In some embodiments, a composition comprising regenerative cells can be sprayed onto the debrided wound bed. In some embodiments, a composition comprising regenerative cells can be dripped or painted onto a debrided wound bed. In some embodiments, the composition comprising regenerative cells is administered systemically, or according to any of the methods of administration discussed herein below. In some embodiments, the composition comprising regenerative cells is administered both locally (e.g., topically or by local injection) and systemically (e.g., intravascularly, intralymphatically, or the like). In some embodiments the regenerative cells are delivered in a simple vehicle such as a physiologic saline or buffered solution. In other embodiments they are delivered in a biologic vehicle such as a fibrin glue. In still further embodiments, the regenerative cells are delivered within the graft. In certain embodiments the regenerative cells are mixed with or delivered in temporal association with other cells types such as keratinocytes and/or dermal cells.

Some embodiments provide methods of reducing the time between wound debridement and application of an autograft to the wound. For example, in some embodiments, method can include application of a composition comprising regenerative cells as disclosed herein to a temporary graft which is applied to the debrided wound. A permanent graft (e.g., an autograft or other type of permanent graft), can be subsequently applied to the debrided wound. In some embodiments, the method includes the step of removing all or part of the graft, prior to application of the autograft. By way of example only, in some embodiments, a composition comprising regenerative cells as disclosed herein can be applied to a graft such as INTEGRA® skin substitute to create a temporary, fortified graft. After a period of time (e.g., 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more) part (or all) of the INTEGRA® graft is removed (e.g., the silicone backing). An autograft is subsequently applied to the wound. Fortification of the INTEGRA® skin substitute with regenerative cells as described herein accelerates vascularization of the wound tissue within and beneath the INTEGRA® skin substitute, and shortens the time required before the wound (e.g., debrided burn) is ready to receive an autograft. In some embodiments, the time required before application of an autograft is reduced by 5%, 10% 15%, 20%, 25%, 30%, 35%, 40%, 45%, 50%, or more. In some embodiments, applying a composition comprising regenerative cells to a skin substitute (e.g., INTEGRA® or the like), reduces the time required before application of an autograft by 12 hours, 1 day, 2 days, 3 days, 4 days, 5 days, 6 days, 7 days, 8 days, 9 days, 10 days, 11 days, 12 days, 13 days, 14 days, or more. In some embodiments, application of the composition comprising regenerative cells improves remodeling of the autograft.

Some embodiments relate to methods of improving healing of autografts. For example, some embodiments disclosed herein relate to a method of improving epithelialization of dispersed or meshed autografts. The method can include the steps of applying a composition comprising regenerative cells as disclosed herein (e.g., a composition comprising adipose-derived regenerative cells or the like), to a meshed autograft or a fully or partially disaggregated suspension of skin cells. In some embodiments, the composition is applied to the meshed autograft or cell suspension to create a fortified graft that is then placed onto a recipient wound bed (e.g., a debrided burn wound or the like). In some embodiments, the meshed autograft or cell suspension is placed onto a recipient wound bed (e.g., a debrided wound or the like) and the composition comprising regenerative cells is applied to the meshed autograft or cell suspension that is already placed in the recipient site. In some embodiments, application of the composition comprising regenerative cells to the meshed autograft or cell suspension improves epithelialization. In some embodiments, application of the composition comprising regenerative cells improves vascularization of the meshed autograft and/or healing wound bed. Epithelialization and vascularization can be readily assessed using any art-accepted methods, including but not limited to, those described in Pomahac, et al. (2007) Regional Anesthesia and Pain Medicine 32(5): 377-381, Greenwood, et al. (2009) J. Plastic Surg. 9: 309-318, and the like. In some embodiments, application of the composition comprising regenerative cells improves remodeling of the meshed autograft. In some embodiments, application of the composition comprising regenerative cells prevents “ghosting” of the graft. As used herein, the term “ghosting” refers to the phenomenon whereby integrated grafts subsequently “dissolve” over time, often as a result of infection. In some embodiments, application of the composition comprising regenerative cells promotes maturation of vessels incorporating into the graft.

Accordingly, provided are embodiments that include the steps of: (1) applying to a graft an effective amount of the compositions including regenerative cells as disclosed herein (e.g., to create a “fortified graft”), (2) contacting the underlying tissue of the wound with the fortified graft; and (3) securing the graft to the underlying tissue, whereby incorporation of the graft into said underlying tissue is promoted. As such, in some embodiments, the regenerative cells can be applied to a skin graft or skin substitute to create a “fortified graft,” which is subsequently administered to a recipient site in a subject in need thereof. In some embodiments, the methods disclosed herein provide for the step of debriding a burn, and administering the fortified graft to the subject. The methods can also include the steps of measuring, analyzing or assessing the incorporation of the fortified graft into the recipient site. In some embodiments, the fortified grafts heal more rapidly than non-fortified grafts. In some embodiments, the subject has radiation injury, e.g., cutaneous radiation injury or acute radiation injury. In some embodiments, the subject has radiation injury and a thermal, chemical, or electrical burn requiring a graft. In some embodiments the subject has an acute or chronic wound arising from a cause other than burn and in which treatment of said wound includes application of a graft.

Also provided are embodiments that include the steps of (1) applying to a recipient wound bed a composition comprising regenerative cells to create a fortified recipient site; and (2) contacting the recipient site with a graft, whereby incorporation of the graft into the recipient wound site is improved. In some embodiments, the subject has radiation injury, e.g., cutaneous radiation injury or acute radiation injury. In some embodiments, the subject has radiation injury and a thermal, chemical, or electrical burn requiring a graft.

In some embodiments, the methods disclosed herein include the step of applying the compositions including regenerative cells disclosed herein to the underlying tissue of a wound (e.g., a debrided burn or wound) topically, or by injection, prior to administration of a graft onto the recipient site, i.e., the underlying tissue of the wound. Accordingly, provided are embodiments that include the steps of: (1) applying to a recipient wound site (e.g., a debrided wound, such as a debrided burn wound, a debrided ulcer, or the like), an effective amount of regenerative cells as disclosed herein, (2) contacting the graft and the underlying tissue of the wound; and (3) securing the graft to the recipient wound site, whereby incorporation of the graft into the recipient wound site is promoted. In some embodiments, the subject has radiation injury, e.g., cutaneous radiation injury or acute radiation injury. In some embodiments, the subject has radiation injury and a thermal, chemical, or electrical burn requiring a graft.

The skilled person will readily appreciate that securing the graft can be accomplished using any acceptable method, including but not limited to, suturing, stapling, gluing (e.g., with a biologically compatible glue such as fibrin or the like), or bandaging.

The methods can include the step of analyzing the graft incorporation into the recipient site. Non-limiting ways to assess graft incorporation include, but are not limited to, those described in Dong, et al. (1993) Ann. Biomed. Eng. 21(1):51-55 (measurement of adherence of graft to the skin surface), Greenhalgh, et al. (1992) J. Burn Care Rehab. 13(3) 334-339 (transcutaneous oxygen and carbon dioxide measurements), as well as other methods, including but not limited to analysis of vascularization and/or necrosis, analysis of the degree of granulation, assessment of wound size (e.g., assessment of epithelialization, assessment of neodermis formation, or both), and the like. In some embodiments, planimetry is used to analyze epithelialization and/or contraction of the recipient site. In some embodiments, administration of the composition comprising regenerative cells improves graft incorporation and healing by increasing vascularization of a graft, by increasing the average lumen size of vessels within the graft, by increasing or accelerating vessel maturation, or the like. Vascularization and lumen size can be readily assessed using art-accepted methods, including histology and the like.

In some embodiments, the methods provided herein prevent or reduce contraction of the wound, e.g., in wounds receiving a fortified graft as discussed herein (regenerative cells and skin graft or skin substitute), or in wounds receiving regenerative cells alone. Accordingly, a subject is identified that has a wound at risk of development of contracture. In some embodiments, the wound at risk of development of contracture is a deep partial thickness wound. In some embodiments, the wound at risk of development of contracture is a full thickness wound. Deep partial thickness and full thickness wounds can be assessed using art-accepted methods described elsewhere herein. In some embodiments, the subject is administered a composition comprising regenerative cells. The composition can be administered systemically, locally, or both. In some embodiments, the wound at risk of development of contracture is contacted with a composition comprising regenerative cells, as described elsewhere herein. In some embodiments, the composition includes a scaffold, e.g., a tissue scaffold (such as adipose tissue or the like). In some embodiments, the composition includes a dermal substitute. In some embodiments, the composition includes a skin graft. Accordingly, the regenerative cells can be mixed with or applied to the surface of, the scaffold. In some embodiments, the composition is applied to the recipient wound site, and a scaffold, e.g., a dermal substitute or skin graft is subsequently applied to the wound site. In some embodiments, the administration of the composition comprising regenerative cells, whether administered systemically or locally, or whether applied in combination with a scaffold or not, slows the rate of contraction of the recipient wound. In some embodiments, the administration of the composition comprising regenerative cells slows the rate of contraction such that the development of contractures is prevented or minimized. In some embodiments, the administration of the composition comprising regenerative cells slows the rate of contraction such that the development of hypertrophic scars is prevented or minimized.

Methods of Preventing, Minimizing, or Treating Hypertrophic Scarring

Provided herein are methods for preventing and/or reducing hypertrophic scarring at a deep-partial thickness or full thickness wound site. The skilled person will readily appreciate that the embodiments disclosed herein are useful in the treatment of a variety of types of wounds involving the placement of a graft to aid in the healing of the wound, e.g., in instances where the area of skin loss is too big to be closed using local skin and stitches alone. For example, the embodiments disclosed herein are useful in the treatment of burns, e.g., including those in which burned tissue is excised. Other exemplary types of wounds in which the methods and compositions disclosed herein are used include, but are not limited to non-healing wounds, e.g., including ischemic wounds and ulcers (for example pressure wounds, wounds and ulcers associated with diabetes, wounds and ulcers associated with peripheral vascular disease, and the like), various traumatic wounds, e.g., caused by mechanical, chemical, insect or other animal sources, and the like. For example, the methods described herein are useful in incorporation of grafts following surgical removal of cancerous, devitalized, or infected tissue.

Methods of preventing or minimizing hypertrophic scarring can include the steps of (1) identifying a subject having a deep partial thickness or full thickness wound; and (2) administering to the deep partial thickness or full thickness wound a composition comprising regenerative cells. In some embodiments, the subject has radiation injury, e.g., cutaneous radiation injury or acute radiation injury. In some embodiments, the subject has radiation injury and a thermal, chemical, or electrical burn or other deep partial thickness or full thickness wound.

In some embodiments, the regenerative cells are applied directly to the wound site. In some embodiments, the regenerative cells are applied in a “fortified graft,” e.g., in combination with a scaffold as described elsewhere herein, including but not limited to fat grafts, skin grafts, or other biological (autologous or non-autologous) or synthetic skin substitutes. In some embodiments, a scaffold is applied to the deep partial thickness or full thickness wound site, and the regenerative cells are subsequently applied to the wound site. In some embodiments, the regenerative cells are mixed together with a scaffold as described herein (e.g., unprocessed adipose tissue or the like), and the mixture is applied to the wound site. In some embodiments, the regenerative cells are mixed together with a scaffold as described herein to produce a fortified scaffold, which is administered to a recipient site, and a skin graft or skin substitute is subsequently applied to the recipient site that has already received the fortified scaffold. In some embodiments, the composition comprising regenerative cells is applied topically to the recipient site. As discussed elsewhere herein, topical administration can include dripping a liquid vehicle comprising the regnerative cells onto the recipient wound site, spraying a vehicle comprising the regenerative cells onto the recipient wound site, or the like. In some embodiments, the composition comprising regenerative cells is injected in or around the wound site (e.g., in a single or multiple injections).

In some embodiments, wherein the wound is a burn, the methods can further include the step of debriding the burn to create a debrided recipient site, and administering the composition comprising regenerative cells, or composition comprising regenerative cells and scaffold (fortified scaffold) to the debrided recipient site of the deep partial thickness or full thickness wound. In some embodiments, a skin graft or dermal substitute is subsequently applied to the recipient site that has received the fortified scaffold, whereby hypertrophic scar formation is prevented or inhibited.

The methods can also include the steps of measuring, analyzing or assessing the formation of hypertrophic scar formation at the wound site.

In some embodiments, the methods disclosed herein include the step of applying the compositions including regenerative cells disclosed herein to the underlying tissue of a wound (e.g., a debrided burn or wound) topically, or by injection, prior to administration of a graft onto the recipient site, i.e., the underlying tissue of the wound. Accordingly, provided are embodiments that include the steps of: (1) applying to a recipient wound site (e.g., a debrided wound, such as a debrided burn wound, a debrided diabetic and/or peripheral vascular disease-associated ulcer wound, a debrided pressure sore, or the like), a composition comprising an effective hypertrophic scar inhibiting amount of regenerative cells as disclosed herein, and (2) securing the graft to the recipient wound site, whereby hypertrophic scar formation is prevented or inhibited.

Some embodiments relate to methods of preventing or slowing wound contraction. In some embodiments, prevention of hypertrophic scarring comprises prevention or slowing wound contraction. Accordingly, in some embodiments, a subject having a deep partial thickness or full thickness wound (e.g., a wound that is at risk of developing hypertrophic scarring if wound contraction proceeds too rapidly), is identified. A composition comprising regenerative cells (e.g., concentrated populations of adipose-derived regenerative cells or the like), can be administered to the subject. In some embodiments, the composition is administered directly to the wound site, e.g., by topical administration or local injection. In some embodiments, the composition is administered systemically, e.g., intravascularly or intralymphatically. In some embodiments, compositions comprising regenerative cells are administered to the subject both locally and systemically. In some embodiments, the methods include the step of measuring wound contraction. Wound contraction can be readily assessed using any method known to those in the art, including, planimetry, e.g., as described in Rogers, et al. (2010) J. Diabetes Sci. Tech, 4(4):799-802. In some embodiments, e.g., wherein the composition comprising regenerative cells is administered locally, the composition includes a scaffold such as a tissue scaffold (e.g., adipose tissue, PRP, or the like), or a biological or biocompatible scaffold (including skin grafts, skin substitutes, and the like). In some embodiments, wound contraction is reduced by more than 3%, 4%, 5%, 6%, 7%, 8%, 9%, 10%, 15%, 20%, or more, by at least 2, 3, 4, 5, 6, 7, 8, 9, 10, 11, 12, 13, 14, 15, 16, 17, 18, 19, 20, or more days post-injury.

Other embodiments provided herein relate to methods of treating hypertrophic scars. that have already developed or that are in the process of developing. For example, treating a hypertrophic scar can refer to minimizing and/or eliminating existing scar tissue, minimizing and/or eliminating hypertrophic scar contracture, improving range of motion over a scarred area, eliminating or minimizing pruritus, improving pliability of hypertrophic scarred tissue, improving firmness of hypertrophic scarred tissue, improving score in one or more art-accepted scar scales (see, e.g., Fearmonti, et al. (2010) J. Plastic Surg. 10: 354-364), decreasing mast cell number and/or myofibroblast cell numbers in hypertrophic scarred tissue, and the like.

A subject having a hypertrophic scar, i.e., a hypertrophic scar that has existed for more than 14 days, more than 30 days, more than 45 days, more than 60 days, more than 90 days, more than 120 days, more than 1 year, more than 5 years, or longer is identified. In some embodiments, the subject has radiation injury, e.g., cutaneous radiation injury or acute radiation injury. In some embodiments, the subject has radiation injury and a hypertrophic scar. In some embodiments, the methods include administration of a composition comprising regenerative cells to a hypertrophic scar, e.g., by local or systemic injection, or any other route of administration described herein. In some embodiments, the regenerative cells are administered with a scaffold, such as the scaffolds described herein below (e.g., an autologous fat graft, an autologous skin graft, allograft, dermal substitute, or any combination thereof, or any other biologic or synthetic scaffold). In some embodiments, the compositions include a scaffold. In some embodiments, the methods include the step of removing the hypertrophic scar tissue using any art accepted method to create a recipient site, and administering the composition comprising regenerative cells to the recipient site. In some embodiments, hypertrophic scar tissue is not removed prior to administering the composition comprising regenerative cells.

In some embodiments relating to minimizing or treating hypertrophic scars, the method includes the step of performing an adjunct treatment or therapy to ameliorate the hypertrophic scar, in combination with the administration of the composition comprising the regenerative cells. For example, in some embodiments, the methods can include, for example, the step of perforating the scar tissue, e.g., as described in U.S. Patent Application Publication No. 2008/0119781, using mechanical force (see, e.g., Costa, et al.: (1999) Mechanical Force Induce Scar Remodeling: Am J Pathol. 155: 1671-1679), surgical removal of the scar tissue (see, e.g., Suzuki, S. (1996): Operation: Operation of keloid and/or hypertrophic scar. 50: 1557-1561), application of a silicone sheet to the lesion (see, e.g., Perkins, et al., (1983) Silicone gel: a new treatment for burn scars and contractures. Burns 9; 201-204), laser and pulsed light treatment of the lesion (see, e. g., Vrijman, et al. (2011) Laser and Intense pulsed Light Therapy for the Treatment of Hypertrophic Scars, British J. Derm. 165(5):934-942), and the like. The skilled artisan will readily appreciate that any adjunct therapy that is performed can be performed prior to, at substantially the same time as, or subsequently to, the administration of the composition comprising regenerative cells. In some embodiments, the composition comprising regenerative cells is a fortified scaffold, and/or fortified graft, as described elsewhere herein that includes regenerative cells in combination with a scaffold or graft as described elsewhere herein.

In some embodiments, the methods include the step of assessing treatment of the hypertrophic scar. For example, in some embodiments, the size of the hypertrophic scar is assessed. In some embodiments, treatment with the compositions comprising regenerative cells as described herein results in a 5%, 10%, 15%, 20%, 25%, 30%, 35%, 40%, 45%, 50%, 55%, 60%, 65%, 70%, 75%, 80%, 85%, 90%, 95%, or more, decrease in scar area. Decrease in scar area can refer to a decrease in the width, height, or depth of the scar as assessed using art-accepted techniques. Non-limiting examples of methods to assess scar area are described, e.g., in Oliviera, et al. (2005) Dermatol. Surg. 31(1): 48-58. In some embodiments, treatment with the compositions comprising regenerative cells as described herein results in a reduction in scar contracture. Accordingly, some methods include the step of assessing the degree or amount of scar contracture. For example, in some embodiments, contracture can be measured by one or more of the methods described in Parry, et al. (2010) J. Burn Care, 31(6): 888-903, or using any number of other art-accepted techniques. In some embodiments, treatment with the compositions comprising regenerative cells as described herein results in a 5%, 10%, 15%, 20%, 25%, 30%, 35%, 40%, 45%, 50%, 55%, 60%, 65%, 70%, 75%, 80%, 85%, 90%, 95%, or more, decrease in scar contracture. In some embodiments, the range of motion over a scarred area is assessed using art-accepted methods. In some embodiments, treatment with the compositions comprising regenerative cells as described herein improves the range of motion by at least 2 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 110 degrees, 120 degrees, or more. Range of motion can be assessed using any art-accepted technique, including but not limited to those described in Palmieri, et al. (2003) J. Burn Care Rehabil. 24:104-108. In some embodiments, pruritus is assessed. In some embodiments, treatment with the compositions comprising regenerative cells results in improvement in pruritus as measured by one or more art-accepted techniques, including but not limited to the tools described in Phan, et al. (2011) Acta Derm. Venereol. 92: 502-507. In some embodiments, pliability of scar tissue is assessed. In some embodiments, treatment of a hypertrophic scar with the compositions comprising regenerative cells as described herein results in improvement of pliability of scar tissue. Pliability can be assessed using any art-accepted technique, including but not limited to those described in Oliviera, et al. (2005) Dermatol. Surg. 31(1): 48-58, Lye et al. (2006) 27(6):82-85, and the like. In some embodiments, bloodflow within the hypertrophic scar is assessed, e.g., using Laser-doppler imaging, or any other art-accepted technique. In some embodiments, elasticity of the scar is assessed. In some embodiments, treatment of a hypertrophic scar with the compositions comprising regenerative cells as described herein results in improvement of elasticity of scar tissue. Elasticity can be assessed using any art-accepted technique, including but not limited to those described in Bartell, et al. (1988) J. Burn Care Rehabil. 9(6): 657-660, and the like. In some embodiments, stiffness of the scar tissue is assessed. In some embodiments, treatment of a hypertrophic scar with the compositions comprising regenerative cells as described herein results in improvement of stiffness of scar tissue. Stiffness can be assessed using any art-accepted technique, including but not limited to those described in McHugh, et al. (1997) J. Burn Care Rehabil. 18(2): 104-108.

Methods of Administration

In some embodiments, the methods disclosed herein include administering a therapeutically effective amount of a composition comprising regenerative cells to a subject. As used herein, the term “therapeutically effective amount” refers to an amount sufficient to mitigate conversion of a burn, and/or to improve graft survival and take. Determination of the exact dose of regenerative cells for the embodiments disclosed herein is well within the ambit of the ordinary skill in the art.

The amount and frequency of administration of the compositions can vary depending on, for example, what is being administered, the state of the patient, and the manner of administration. In therapeutic applications, compositions can be administered to a patient suffering from a burn (e.g., a subject that has been identified as having a partial thickness burn and/or a full thickness burn or that is in need of a graft), in an amount sufficient to relieve or least partially mitigate burn progression. The compositions can also be administered to a patient receiving a graft (e.g., a subject that has a debrided wound or burn) in an amount sufficient to improve survival of the graft, once administered to the patient. The dosage is likely to depend on such variables as the type and extent of the burn graft, as well as the age, weight and general condition of the particular subject, and the route of administration. Effective doses can be extrapolated from dose-response curves derived from in vitro or animal model test system.

In some embodiments, at least 1×10² regenerative cells is a therapeutically effective amount. In some embodiments, at least 1×10³ regenerative cells is a therapeutically effective amount. In some embodiments, at least 1×10⁴ cells is a therapeutically effective amount. In some embodiments, at least 1×10⁵ regenerative cells is a therapeutically effective amount. In some embodiments, at least 1×10⁶ regenerative cells is a therapeutically effective amount. In some embodiments, at least 1×10⁷ regenerative cells is a therapeutically effective amount. In some embodiments, at least 1×10⁸ regenerative cells is a therapeutically effective amount. In some embodiments, at least 1×10⁹ regenerative cells is a therapeutically effective amount. In some embodiments, at least 1×10¹⁰ regenerative cells is a therapeutically effective amount. In some embodiments, a greater number of regenerative cells is therapeutically effective to treat burns with a larger surface area than to treat burns with a smaller surface area. In some embodiments, a greater number of regenerative cells is therapeutically effective to treat deeper burns than to treat burns that are not as deep (e.g., a greater number of regenerative cells may be therapeutically effective to treat a deep partial thickness wound than to treat a superficial partial thickness wound). In some embodiments, a greater number of regenerative cells is therapeutically effective to improve the survival or take of a graft that has a larger surface area, compared to a smaller graft. In some embodiments, the number of regenerative cells that is therapeutically effective depends upon whether the graft is a full thickness or split-thickness skin graft, or whether the graft is a skin substitute or other synthetic or biological scaffold.

In some embodiments, the regenerative cells comprise at least 0.05% stem cells. For example, in some embodiments, the regenerative cells comprise at least 0.1%, 0.2%, 0.3%, 0.4%, 0.5%, 0.6%, 0.7%, 0.8%, 0.9%, 1.0%, 2%, 3%, 4%, 5%, 10%, 15%, 20%, 25%, 50%, or more, stem cells. That is, in some embodiments, at least 0.1%, 0.2%, 0.3%, 0.4%, 0.5%, 0.6%, 0.7%, 0.8%, 0.9%, 1.0%, 2%, 3%, 4%, 5%, 10%, 15%, 20%, 25%, 50%, or more, of the nucleated cells within the regenerative cell population are stem cells.

### Regenerative Cells

In the embodiments disclosed herein, regenerative cells are used for mitigating and/or prevent burn progression/conversion. In various embodiments, regenerative cells are used for improving the take or viability of grafts, and or promoting the healing of grafts. As mentioned above, a population of “regenerative cells” disclosed herein can be a homogeneous or heterogeneous population of cells that cells that which cause or contribute to complete or partial regeneration, restoration, or substitution of structure or function of an organ, tissue, or physiologic unit or system to thereby provide a therapeutic, structural or cosmetic benefit. Examples of regenerative cells include, but are not limited to adult stem cells, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes.

The regenerative cells disclosed herein can be isolated from various tissues, including, but not limited to bone marrow, placenta, adipose tissue, skin, eschar tissue, endometrial tissue, adult muscle, corneal stroma, dental pulp, Wharton's jelly, amniotic fluid, and umbilical cord. The regenerative cells disclosed herein can be isolated from the tissues above using any means known to those skilled in the art or discovered in the future.

By way of example only, regenerative cells can be isolated from adipose tissue by a process wherein tissue is excised or aspirated. Excised or aspirated tissue can be washed, and then enzymatically or mechanically disaggregated in order to release cells bound in the adipose tissue matrix. Once released, the cells can be suspended. By way of example only, regenerative cells useful in the embodiments disclosed herein can be isolated using the methods and/or devices described in U.S. Pat. Nos. 7,390,484; 7,585,670, 7,687,059, 8,309,342, 8,440,440, US Patent Application Publication No's. 2013/0164731, 2013/0012921, 2012/0164113, US2008/0014181. International Patent Application Publication No. WO2009/073724, WO/2013030761, and the like, each of which is herein incorporated by reference.

Exemplary, non-limiting methods for isolation of regenerative cells from bone marrow useful in the embodiments disclosed herein are described in U.S. Pat. No. 5,879,940, U.S. Patent Application Publication Nos. 2013/0101561, 2013/0266541 European Patent Application Publication No. EP2488632A1, EP0241578A2, EP2624845A2, International Patent Application Publication No. WO2011047289A1, WO1996038482A, each of which is herein incorporated by reference.

Exemplary, non-limiting methods for isolation of regenerative cells from placental tissue useful in the embodiments disclosed herein are described in U.S. Pat. No. 8,580,563, U.S. Patent Application Publication No. 20130040281, International Patent Application Publication No. WO2003089619A, Klein, et al. (2011) Methods Mol Biol. 698:75-88, Vellasamy, et al. (2012) World J Stem Cells 4(6): 53-61; Timmins, et al. (2012) Biotechnol Bioeng. 109(7):1817-26; Semenov, et al. (2010) Am J Obstet Gynecol 202:193-e.13, and the like, each of which is herein incorporated by reference.

Exemplary, non-limiting methods for isolation of regenerative cells from skin useful in the embodiments disclosed herein are described in Toma, et al. (2001), Nat Cell Biol. 3(9):778-84; Nowak, et al. (2009), Methods Mol Biol. 482:215-32; U.S Patent Application Publication No. 2007/0248574, and the like, each of which is herein incorporated by reference.

Exemplary, non-limiting methods for isolation of regenerative cells from eschar tissue useful in the embodiments disclosed herein are described in Van der Veen, et al. (2012), Cell Transplant. 21(5):933-42, and elsewhere herein below.

Exemplary, non-limiting methods for isolation of regenerative cells from endometrial tissue useful in the embodiments disclosed herein are described in U.S. Patent Application Publication No. 2013/0156726, 2008/0241113, and the like, each of which is herein incorporated by reference in its entirety.

Exemplary, non-limiting methods for isolation of regenerative cells from muscle tissue useful in the embodiments disclosed herein are described in U.S. Pat. No. 6,337,384, U.S. Patent Application Publication No. 2001/019966, 2011/0033428, 2005/0220775, and the like, each of which is herein incorporated by reference.

Exemplary, non-limiting methods for isolation of regenerative cells from corneal tissue useful in the embodiments disclosed herein are described in U.S. Patent Application Publication No. 2005084119, Sharifi, et al. (2010) Biocell. 34(1):53-5, and the like, each of which is herein incorporated by reference.

Exemplary, non-limiting methods for isolation of regenerative cells from dental pulp useful in the embodiments disclosed herein are described in U.S. Patent Application Publication No. 2012/0251504, Gronthos, et al. (2011) Methods Mol Biol. 698:107-21; Suchanek, et al. Acta Medica (Hradec Kralove). 2007; 50(3):195-201; Yildirm, Sibel, “Isolation Methods of Dental Pulp Stem Cells,” in Dental Pulp Stem Cells: Springer Briefs in Stem Cells, pp. 41-51, © 2013, Springer New York, New York, N.Y., and the like, each of which is herein incorporated by reference.

Exemplary, non-limiting methods for isolation of regenerative cells from Wharton's jelly useful in the embodiments disclosed herein are described in U.S. Patent Application Publication Nos. 2013/0183273, 2011/0151556, International Patent Application Publication No. WO 04/072273A1, Sheshareddy, et al. (2008) Methods Cell Biol. 86:101-19, Mennan, et al. (2013) BioMed Research International, Article ID 916136, Corotchi, et al. (2013) Stem Cell Research & Therapy 4:81, and the like, each of which is herein incorporated by reference.

Exemplary, non-limiting methods for isolation of regenerative cells from amniotic fluid useful in the embodiments described herein are described in U.S. Pat. No. 8,021,876, International Patent Application Publication No. WO 2010/033969A1, WO 2012/014247A1, WO 2009/052132, U.S. Patent Application Publication No. 2013/0230924, 2005/0054093, and the like, each of which is herein incorporated by reference.

Exemplary, non-limiting methods for isolation of regenerative cells from the umbilical cord useful in the embodiments described herein are described in U.S. Patent Application Publication No. 20130065302, Reddy, et al. (2007), Methods Mol Biol. 407:149-63, Hussain, et al. (2012) Cell Biol Int. 36(7):595-600, Pham, et al. (2014) Journal of Translational Medicine 2014, 12:56, Lee, et al. (2004) Blood 103(5): 1669-1675, and the like, each of which is herein incorporated by reference.

The regenerative cells in the methods and compositions described herein can be a heterogeneous population of cells that includes stem and other regenerative cells. In some embodiments, the regenerative cells in the methods and compositions described herein can include stem and endothelial precursor cells. In some embodiments, the regenerative cells can include stem and pericyte cells. In some embodiments, the regenerative cells can include stem cells and leukocytes. For example, in some embodiments, the regenerative cells can include stem cells and macrophages. In some embodiments, the regenerative cells can include stem cells and M2 macrophages. In some embodiments, the regenerative cells can include pericytes and endothelial precursor cells. In some embodiments, the regenerative cells can include platelets. Preferably, the regenerative cells comprise stem cells and endothelial precursor cells. In some embodiments, the regenerative cells can include regulatory cells such as Treg cells.

In some embodiments, the regenerative cells are adipose-derived. Accordingly, some embodiments provide methods and compositions for mitigating or reducing burn progression with adipose-derived regenerative cells, e.g., that include adipose-derived stem and endothelial precursor cells.

In some embodiments, the regenerative cells are not cultured prior to use. By way of example, in some embodiments, the regenerative cells are for use following isolation from the tissue of origin, e.g., bone marrow, placenta, adipose tissue, skin, eschar tissue, endometrial tissue, adult muscle, cornea stroma, dental pulp, Wharton's jelly, amniotic fluid, umbilical cord, and the like.

In some embodiments, the regenerative cells are cultured prior to use. For example, in some embodiments, the regenerative cells are subjected to “limited culture,” i.e., to separate cells that adhere to plastic from cells that do not adhere to plastic. Accordingly, in some embodiments, the regenerative cells are “adherent” regenerative cells. An exemplary, non-limiting method of isolating adherent regenerative cells from adipose tissue are described e.g., in Zuk, et al. (2001). Exemplary, non-limiting method of isolating adherent regenerative cells from bone marrow are described, e.g., Pereira (1995) Proc. Nat. Acad. Sci. USA 92:4857-4861, Castro-Malaspina et al. (1980), Blood 56:289-30125; Piersma et al. (1985) Exp. Hematol. 13:237-243; Simmons et al., 1991, Blood 78:55-62; Beresford et al., 1992, J. Cell. Sci. 102:341-3 51; Liesveld et al. (1989) Blood 73:1794-1800; Liesveld et al., Exp. Hematol 19:63-70; Bennett et al. (1991) J. Cell. Sci. 99:131-139), U.S. Pat. No. 7,056,738, and the like.

In some embodiments, the regenerative cells are cultured for more than 3 passages in vitro. For example, in some embodiments, the regenerative cells are cultured for 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more passages in vitro.

The regenerative cells described herein can be cultured according to approaches known in the art, and the cultured cells can be used in several of the embodied methods. For example, regenerative cells can be cultured on collagen-coated dishes or 3D collagen gel cultures in endothelial cell basal medium in the presence of low or high fetal bovine serum or similar product, as described in Ng, et al., (2004), Microvasc. Res. 68(3):258-64, incorporated herein by reference. Alternatively, regenerative cells can be cultured on other extracellular matrix protein-coated dishes. Examples of extracellular matrix proteins that may be used include, but are not limited to, fibronectin, laminin, vitronectin, and collagen IV. Gelatin or any other compound or support, which similarly promotes adhesion of endothelial cells into culture vessels may be used to culture regenerative cells, as well.

Examples of basal culture medium that can be used to culture regenerative cells in vitro include, but are not limited to, EGM, RPMI, M199, MCDB131, DMEM, EMEM, McCoy's 5A, Iscove's medium, modified Iscove's medium, or any other medium known in the art to support the growth of blood endothelial cells. In some embodiments, the regenerative cells are cultured in EGM-2MV media. Examples of supplemental factors or compounds that can be added to the basal culture medium that could be used to culture regenerative cells include, but are not limited to, ascorbic acid, heparin, endothelial cell growth factor, endothelial growth supplement, glutamine, HEPES, Nu serum, fetal bovine serum, human serum, equine serum, plasma-derived horse serum, iron-supplemented calf serum, penicillin, streptomycin, amphotericin B, basic and acidic fibroblast growth factors, insulin-growth factor, astrocyte conditioned medium, fibroblast or fibroblast-like cell conditioned medium, sodium hydrogencarbonate, epidermal growth factor, bovine pituitary extract, magnesium sulphate, isobutylmethylxanthine, hydrocortisone, dexamethasone, dibutyril cyclic AMP, insulin, transferrin, sodium selenite, oestradiol, progesterone, growth hormone, angiogenin, angiopoietin-1, Del-1, follistatin, granulocyte colony-stimulating factor (G-CSF), erythropoietin, hepatocyte growth factor (HGF)/scatter factor (SF), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular endothelial growth factor (VEGF)/vascular permeability factor (VPF), interleukin-3 (IL-3), interleukin 7 (IL-7), interleukin-8 (IL-8), ephrins, matrix metalloproteinases (such as MMP2 and MMP9), or any other compound known in the art to promote survival, proliferation or differentiation of endothelial cells.

Further processing of the cells may also include: cell expansion (of one or more regenerative cell types) and cell maintenance (including cell sheet rinsing and media changing); sub-culturing; cell seeding; transient transfection (including seeding of transfected cells from bulk supply); harvesting (including enzymatic, non-enzymatic harvesting and harvesting by mechanical scraping); measuring cell viability; cell plating (e.g., on microtiter plates, including picking cells from individual wells for expansion, expansion of cells into fresh wells); high throughput screening; cell therapy applications; gene therapy applications; tissue engineering applications; therapeutic protein applications; viral vaccine applications; harvest of regenerative cells or supernatant for banking or screening, measurement of cell growth, lysis, inoculation, infection or induction; generation of cell lines (including hybridoma cells); culture of cells for permeability studies; cells for RNAi and viral resistance studies; cells for knock-out and transgenic animal studies; affinity purification studies; structural biology applications; assay development and protein engineering applications.

In some embodiments, methods for isolating regenerative useful in the embodiments described herein can include positive selection (selecting the target cells), negative selection (selective removal of unwanted cells), or combinations thereof. In addition to separation by flow cytometry as described herein and in the literature, cells can be separated based on a number of different parameters, including, but not limited to, charge or size (e.g., by dielectrophoresis or various centrifugation methods, etc.).

By way of example, the regenerative cells useful in the methods of treatment disclosed herein may be identified by different combinations of cellular and genetic markers. For example, in some embodiments, the regenerative cells express CD90. In some embodiments, the regenerative cells do not express significant levels of lin. In some embodiments, the regenerative cells do not express significant levels of ckit. In some embodiments, the regenerative cells are CD90+/lin-/ckit-/CD45−.

In some embodiments, the regenerative cells express STRO-1. In some embodiments, the regenerative cells express STRO-1 and CD49d. In some embodiments, the regenerative cells express STRO-1, CD49d, and one or more of CD29, CD44, CD71, CD90, C105/SH2 and SH3. In some embodiments, the regenerative cells express STRO-1, CD49d, and one or more of CD29, CD44, CD71, CD90, C105/SH2 and SH3, but express low or undetectable levels of CD106.

In some embodiments, the regenerative cells express one or more of STRO-1, CD49d, CD13, CD29, SH3, CD44, CD71, CD90, and CD105, or any combination thereof. By way of example only, in some embodiments, the regenerative cells express each of do not express significant levels of CD31, CD34, CD45 and CD104 and do not express detectable levels of CD4, CD8, CD11, CD14, CD16, CD19, CD33, CD56, CD62E, CD106 and CD58.

In some approaches, the regenerative cells are CD14 positive and/or CD11b positive. In some embodiments, the cells are depleted for cells expressing the markers CD45(+). In some embodiments, the cells are depleted for cells expressing glycophorin-A (GlyA). In some embodiments, the cells are depleted for CD45(+) and GlyA(+) cells.

Negative selection of cells, e.g., depletion of certain cell types from a heterogeneous population of cells can done using art-accepted techniques, e.g., utilizing micromagnetic beads or the like. In some embodiments, the regenerative cells are CD34+.

In some embodiments, the regenerative cells are not cryopreserved. In some embodiments, the regenerative cells are cryopreserved. For example, in some embodiments, the regenerative cells include cryopreserved cells, e.g., as described in Liu, et al. (2010) Biotechnol Prog. 26(6):1635-43, Carvalho, et al. (2008) Transplant Proc.; 40(3):839-41, International Patent Application Publication No. WO 97/039104, WO 03/024215, WO 2011/064733, WO 2013/020492, WO 2008/09063, WO 2001/011011, European Patent No. EP0343217 B1, and the like.

In some embodiments, regenerative cells are isolated from a subject having radiation injury, e.g., cutaneous or acute radiation injury. Some of the embodiments described herein are based, in part, upon the surprising discovery that populations of regenerative cells isolated from the adipose tissue of subjects with radiation injury have similar properties (e.g., cell type, cell viability, cell frequency, and cell function), as regenerative cells isolated from adipose tissue of subjects with no radiation injury.

In some embodiments, the regenerative cells are isolated from adipose tissue obtained from eschar. For example, in some embodiments, the regenerative cells are isolated from adipose tissue obtained from tangential or en bloc escharectomy. The embodiments disclosed herein are based, in part, upon the discovery that regenerative cell populations isolated from adipose tissue obtained from eschar have similar properties (e.g., cell type, cell viability, cell frequency, and cell function), as regenerative cells isolated from non-eschar adipose tissue.

Scaffolds

In some embodiments, the regenerative cells disclosed herein can be administered to a subject with a scaffold. In some embodiments, the scaffold can be a skin substitute, e.g., a biological or synthetic skin substitute. Exemplary skin substitutes useful in the embodiments disclosed herein include, but are not limited to, cell-containing skin substitutes such as EPICEL® skin graft (Genzyme Biosurgery, MA, USA); CELLSPRAY® skin graft (Avita Medical, Perth, Australia), MYSKIN™ skin graft (CellTran Ltd., Sheffeild, UK), LASERSKIN® skin graft (Fidia Advanced Biopolymers, Abano Terme, Italy); RECELL® skin graft (Avita Medical, Perth, Australia), ORCEL® skin graft (Ortec Int'l, GA, USA), APLIGRAFT® skin graft (Organogenesis, MA, USA), POLYACTIVE® skin graft (HC Implants BV, Leiden, Netherlands), and the like. Exemplary non-cellular skin substitutes useful in the embodiments disclosed herein include, but are not limited INTEGRA® (Integra NeuroSciences, NJ, USA) scaffold; ALLODERM® scaffold (LifeCell Corp., NJ, USA), HYALOMATRIX PA® scaffold (Fidia Advanced Biopolymers, Abano Terme, Italy), DERMAGRAFT® scaffold (Advanced BioHealing, CT, USA), TRANSCYTE® (Advanced BioHealing, CT, USA), HYALOGRAFT 3D™ scaffold (Fidia Advanced Biopolymers, Abano Terme, Italy), DERMAMATRIX® scaffold (Synthes, CMF, PA, USA), and the like. The skilled person will readily appreciate skin substitutes (whether cellular or acellular) developed in the future are useful in the embodiments disclosed herein. Various skin substitutes useful in the embodiments disclosed herein are described in US Patent Application Publication number U.S. 2011/0245929.

Other, non-limiting examples of scaffolds and matrices useful in the embodiments disclosed herein include PURAPLY® collagen dressing (Organogensis, Inc. MA, USA), ALLEVYN® matrix (Smith & Nephew, Hull, UK), ACTICOAT® matrix (Smith & Nephew, Hull, UK), CICA-CARE® matrix (Smith & Nephew, Hull, UK), DURA-FIBER® matrix (Smith & Nephew, Hull, UK), INTRASITE® matrix (Smith & Nephew, Hull, UK), IODOSORB® matrix (Smith & Nephew, Hull, UK), OPSITE® matrix (Smith & Nephew, Hull, UK), PROFORE® matrix (Smith & Nephew, Hull, UK), CUTINOVA® matrix (Smith & Nephew, Hull, UK), JELONET® matrix (Smith & Nephew, Hull, UK), BIOBRANE® matrix (Smith & Nephew, Hull, UK) FORTAFLEX® bioengineered collagen matrix (Organogenesis, MA, USA), FORTAGEN® collagen construct (Organogenesis, MA, USA), and the like.

Accordingly, in some embodiments, the regenerative cells are combined with a biocompatible matrix such as a mesh, a gauze, a sponge, a monophasic plug, a biphasic plug, a paste, a putty, a wrap, a bandage, a patch, a mesh, or a pad. In some embodiments, the biocompatible matrix can be resorbable, porous, or both resorbable and porous. Biocompatible matrices useful in the embodiments disclosed herein can include one or more of the following: proteins, polysaccharides, nucleic acids, carbohydrates, inorganic components or minerals, and synthetic polymers; or mixtures or combinations thereof. For example, in some embodiments, the biocompatible matrix can include one or more of a polyurethane, e.g., NOVOSORB™ biocompatible polyurethane matrices, a siloxane, a polysiloxane, a collagen, a glycosaminoglycan, oxidized regenerated cellulose (ORC), an ORC:collagen composite, an alginate, an alginatexollagen composite, a ethylene diamine tetraacetic acid (EDTA),a poly(lactic-co-glycolitic acid (PLGA), a carboxymethylcellulose, a granulated collagen-glycosaminoglycan composite, methylcellulose, hydroxypropyl methylcellulose, or hydroxyethyl cellulose alginic acid, poly(a-hydroxy acids), poly(lactones), poly(amino acids), poly(anhydrides), poly(orthoesters), poly(anhydride-co-imides), poly(orthocarbonates), poly(a-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PUB), poly(e-caprolactone), poly(5-valerolactone), poly(y-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), polyvinyl alcohol), poly(vinylpyrrolidone), poly(ethyloxazoline), poly(ethylene oxide)-co-poly(propylene oxide) block copolymers, poly(ethylene terephthalate)polyamidearabic gum, guar gum, xantham gum, gelatin, chitin, chitosan, chitosan acetate, chitosan lactate, chondroitin sulfate, N,O-carboxymethyl chitosan, a dextran, fibrin glue, glycerol, hyaluronic acid, sodium hyaluronate, a cellulose, a glucosamine, a proteoglycan, a starch, lactic acid, a pluronic, sodium glycerophosphate, glycogen, a keratin, a silk, one or more composites thereof, one or more mixtures thereof, or one or more combinations thereof. In some embodiments, comprises calcium phosphate.

In some embodiments, the biocompatible matrix may comprise a collagen. In certain embodiments, the biocompatible matrix comprises a Type I collagen, a Type II collagen, a Type III collagen, a Type IV collagen, a Type V collagen, a Type VI collagen, a Type VII collagen, a Type VIII collagen, or combinations thereof. Moreover, the collagen can comprise bovine collagen, human collagen, porcine collagen, equine collagen, avian collagen, or combinations thereof. In certain embodiments, the collagen comprises bovine Type I collagen or human Type I collagen. In some embodiments the collagen is in combination with other materials (e.g., chondroitin 6 sulfate) and/or is supplemented with materials that provide barrier function (e.g., a silicone backing vapor barrier). One example of a composite collagen-containing graft is INTEGRA® (Integra NeuroSciences, NJ, USA) scaffold.

In some embodiments, the regenerative cells can be combined with a HELISTAT® absorbable collagen hemostatic sponge (Integra Life Sciences, NJ, USA); a HELITENE absorbable collagen hemostatic agent (Integra Life Sciences, NJ, USA); Matrix Collagen Particles™ wound dressing (Collagen Matrix, Inc., NJ, USA); Matrix Collagen Sponge™ wound dressing (Collagen Matrix, Inc., NJ, USA); OASIS® wound matrix (Smith & Nephew, Hull, UK); BIOBLANKET™ surgical mesh (Kensey Nash, Corp.); ZIMMER™ collagen repair patch (Zimmer, Inc., Swisdon, UK); PROMOGRAN™ matrix wound dressing (Systagenix, MA, USA), FIBROCOL PLUS® collagen dressing (Systagenix, MA, USA), or the like. Yet other scaffolds and grafts useful in the embodiments disclosed herein are described in U.S. Pat. No. 6,979,670, 7,972,631, 7,824,711, and 7,358,284U.S. Patent Application Publication No. 2011/0091515, and the like.

In some embodiments, the regenerative cells are combined with a tissue scaffold, e.g., unprocessed adipose tissue, platelet rich plasma, or other tissue. Mixture of regenerative cells with tissue to form a fortified scaffold (e.g., a cell-enriched fat graft) useful in the embodiments described herein is disclosed, for example, in U.S. Pat. No. 7,651,684, and Kakudo, et al. (2013) Journal of Translational Medicine 11:254, and the like.

Combination Therapy

As explained in further detail below, some embodiments provide for treatment of subjects with combination therapy, i.e., one or more additional additives (e.g., pharmaceutical agents, biologic agents, or other therapeutic agents) in addition to the regenerative cells as described herein.

In some embodiments, the one or more additional “agents” described above can be administered in a single composition with the regenerative. In some embodiments, the one or more additional “agents” can be administered separately from the regenerative cells. For example, in some embodiments, one or more additional agents can be administered just prior to, or just after, administration of the regenerative cells. As used herein, the term “just prior” can refer to within 15 minutes, 30 minutes, an hour, 2 hours, 3 hours, 4 hours, 5 hours, or the like. Likewise, the phrase “just after administration” can refer to within 15 minutes, 30 minutes, an hour, 2 hours, 3 hours, 4 hours, 5 hours, or the like.

Additional agents useful in combination therapy in the methods described herein include, for example, growth factors, cytokines, platelet rich plasma, steroids, non-steroidal anti-inflammatory agents, anti-bacterial and anti-fungal agents, as well as other agents known in the art to have beneficial effects in treatment of burn.

1) Growth Factors, Cytokines, and Hormones

Various growth factors, cytokines, and hormones have been shown to have beneficial effects, e.g., in re-epithelialization and recovery in burn injury. See, e.g., Wenczak, et al. (1992) J. Clin. Invest. 90:2392-2401.

In some embodiments, subjects can be administered one or more growth factors, cytokines or hormones, including combinations thereof, in addition to the regenerative cells disclosed herein. For example, in some embodiments, growth factors are administered concomitantly with, prior to, or following the administration of the regenerative cells. Non-limiting examples of growth factors useful in the embodiments disclosed herein include, but are not limited to, angiogenin, angiopoietin-1 (Ang-1), angiopoietin-2 (Ang-2), brain-derived neurotrophic factor (BDNF), Cardiotrophin-1 (CT-1), ciliary neurotrophic factor (CNTF), Del-1, acidic fibroblast growth factor (aFGF), basic fibroblast growth factor (bFGF), follistatin, ganulocyte colony-stimulating factor (G-CSF), glial cell line-derived neurotrophic factor (GDNF), hepatocyte growth factor (HGF), scatter factor (SF), Interleukin-8 (IL-8), leptin, midkine, nerve growth factor (NGF), neurotrophin-3 (NT-3), Neurotrophin-4/5, Neurturin (NTN), placental growth factor, Platelet-derived endothelial cell growth factor (PD-ECGF), Platelet-derived growth factor-BB (PDGF-BB), Pleiotrophin (PTN), Progranulin, Proliferin, PBSF/SDF-1, Transforming growth factor-alpha (TGF-alpha), Transforming growth factor-beta (TGF-beta), Tumor necrosis factor-alpha (TNF-alpha), Vascular endothelial growth factor (VEGF), vascular permeability factor (VPF), erythropoietin (see, e.g., Tobalem, et a. (2012) Br. J. Surg. 99(9):1295-1303), and the like.

2) Anti-Inflammatory Agents

In some embodiments, subjects are administered on or more anti-inflammatory agents, in addition to the regenerative cells as disclosed herein. As used herein, the term “anti-inflammatory agent” refers to any compound that reduces inflammation, and includes, but is not limited to steroids, non-steroidal anti-inflammatory drugs, and other biologics that have been demonstrated to have an anti-inflammatory effect.

Accordingly, in some embodiments, steroids are administered concomitantly with, prior to, or following the administration of the regenerative cells. Non-limiting examples of steroids useful in the embodiments disclosed herein include, but are not limited to, progestegens, e.g., progesterone, and the like; corticosteroids, e.g., prednisone, aldosterone, cortisol, and the like, androgens, e.g., testosterone, and the like, and estrogens.

Other anti-inflammatory agents useful in the embodiments disclosed herein include, for example, antibodies that inhibit action of TNF-α, IL-6 (see, e.g., Sun, et al. (2012) Repair and Regeneration, 20(4): 563-572), anti-TNF conjugates, Sun, et al. (2012) Wound Repair Regen. 20(4): 563-572, and the like. These anti-inflammatory agents have been demonstrated to exhibit beneficial effects in burn recovery.

Non-steroidal anti-inflammatory drugs useful in the embodiments disclosed herein s include propionic derivatives; acetic acid derivatives; biphenylcarboxylic acid derivatives; fenamic acid derivatives; and oxicams. Examples of anti-inflammatory actives include without limitation acetaminophen, diclofenac, diclofenac sodium and other salts, ibuprofen and its salts acetaminophen, indomethacin, oxaprozin, pranoprofen, benoxaprofen, bucloxic acid, elocon; and mixtures thereof.

3) Anti-Oxidants

Anti-oxidants have been shown to be useful in recovery from burn injury. See, e.g., F. H. Al-Jawad, et al. (2008) Ann Burns Fire Disasters 21(4): 186-191. Accordingly, in some embodiments, the methods and compositions disclosed herein include administration of one or more anti-oxidants in addition to the regenerative cells. Antioxidants useful in the embodiments disclosed herein include, but are not limited to, N-acetylcysteine, curcumarin, galactomannan, pyruvate and other alpha-ketoacids, thioglycollate vitamin A and derivatives, including retinoic acid, retinyl aldehyde, retin A, retinyl palmitate, adapalene, and beta-carotene; vitamin B (panthenol, provitamin B5, panthenic acid, vitamin B complex factor); vitamin C (ascorbic acid and salts thereof) and derivatives such as ascorbyl palmitate; vitamin D including calcipotriene (a vitamin D3 analog) vitamin E including its individual constituents alpha-, beta-, gamma-, delta-tocopherol and cotrienols and mixtures thereof and vitamin E derivatives including vitamin E palmitate, vitamin E linolate and vitamin E acetate; vitamin K and derivatives; vitamin Q (ubiquinone) and any combination thereof.

4) Platelet-Containing Fluids

Platelet rich plasma (“PRP”) has been demonstrated to have beneficial effects following burn injury. See, e.g., Pallua, et al. (2010) Burns, 36(1):4-8. Accordingly, in some embodiments, subjects are administered platelet rich plasma, in addition to the regenerative cells disclosed herein. For example, in some embodiments, a platelet containing fluid is administered concomitantly with, prior to, or following the administration of the regenerative cells. In some embodiments, the regenerative cells as disclosed herein are combined with a synergistically effective amount of platelet-containing fluid.

As used herein, the term “platelet-containing fluid” refers to any fluid, either biological or artificial, which contains platelets. Non-limiting examples of such fluids include various forms of whole blood, blood plasma, platelet rich plasma, concentrated platelets in any medium, or the like, derived from human and non-human sources. For example, in some embodiments, the platelet-containing fluid refers to blood, platelets, serum, platelet concentrate, platelet-rich plasma (PRP), platelet-poor plasma (PPP), plasma, fresh frozen plasma (FFP), and the like.

The term “PRP” as used herein refers to a concentration of platelets greater than the peripheral blood concentration suspended in a solution of plasma. Methods for isolating PRP useful in the embodiments disclosed herein are known in the art. See, e.g., U.S. Pat. No. 8,557,535, International Patent Application Publication No. WO 09/155069, U.S. Patent Application Publication Nos., US20100183561, US20030060352, US20030232712, US20130216626, US20130273008, US20130233803, US20100025342, European Patent No. EP1848474B1, and the like. Platelets or PRP can suspended in an excipient other than plasma. In some embodiments, the platelet composition can include other excipients suitable for administration to a human or non-human animal including, but not limited to isotonic sodium chloride solution, physiological saline, normal saline, dextrose 5% in water, dextrose 30% in water, lactated ringer's solution and the like. Typically, platelet counts in PRP as defined herein range from 500,000 to 1,200,000 per cubic millimeter, or even more. PRP may be obtained using autologous, allogeneic, or pooled sources of platelets and/or plasma. PRP may be obtained from a variety of animal sources, including human sources. In preferred embodiments, PRP according to the invention is buffered to physiological pH.

Methods of Administration

Compositions administered according to the methods described herein can be introduced into the subject by, e.g., by intravenous, intra-arterial, intradermal, intramuscular, intra-lymphatic, intranodal, intramammary, intraperitoneal, intrathecal, retrobulbar, intrapulmonary (e.g., term release); by oral, sublingual, nasal, anal, vaginal, or transdermal delivery, or by surgical implantation at a particular site. The introduction may consist of a single dose or a plurality of doses over a period of time. In such cases the plurality of introductions need not be by the same mechanism. For example, in some embodiments introduction at one time might be in the form of a topical spray of the regenerative cells whereas at another time the introduction may be regenerative cells combined with an autologous fat graft. Vehicles for cell therapy agents are known in the art and have been described in the literature. See, for example Remington's Pharmaceutical Sciences, 18th Ed. (1990, Mack Publ. Co, Easton Pa. 18042) pp 1435-1712, incorporated herein by reference. Sterile solutions are prepared by incorporating the regenerative cells that in the required amount in the appropriate buffer with or without various of the other components described herein.

In some embodiments, the regenerative cells described herein can be administered directly to the burn. For example, in some embodiments, the regenerative cells disclosed herein are formulated for injection. Accordingly, in some embodiments, the compositions disclosed herein are formulated for intravenous, intraarterial, intradermal, intramuscular, intraperitoneal, intrasternal, subcutaneous, intranodal and intra-lymphatic injection, infusion, and placement. In some embodiments, the compositions disclosed herein are formulated for intra-lymphatic delivery. Accordingly, in some embodiments, the regenerative cells can be injected into the burn site, e.g., within the zone of coagulation, the zone of stasis, or the zone of hyperemia of a burn (subcutaneously, intramuscularly, or the like).

In some embodiments, the regenerative cells disclosed herein injected via subcutaneous or intramuscular injection, adjacent to the zone of coagulation. In some embodiments, the regenerative cells disclosed herein are injected adjacent to the zone of stasis. In some embodiments, the regenerative cells disclosed herein are injected into and adjacent to the zone of coagulation. In some embodiments, the regenerative cells disclosed herein are injected into and adjacent to the zone of stasis. Accordingly, in some embodiments, the regenerative cells are formulated for administration in multiple doses, e.g., in multiple injections in and/or around the burn. In some embodiments, the number of injections depends upon the size of the burn. For example, in some embodiments, as the area (and/or the severity) of the burn increases, a greater the number of injections of the regenerative cells is provided. In some embodiments, for example, the regenerative cells as disclosed herein are injected into and around the burn every 0.1 mm², 0.2 mm², 0.3 mm², 0.4 mm², 0.5 mm², 0.6 mm², 0.7 mm², 0.8 mm², 0.9 mm², 1.0 mm², 2 mm², 3 mm², 4 mm², 5 mm², 6 mm², 7 mm², 8 mm², 9 mm², 10 mm², 20 mm², 30 mm², 40 mm², 50 mm², 60 mm², 70 mm², 80 mm², 90 mm², 1 cm², 5 cm², 10 cm², 20 cm², 30 cm², 40 cm², 50 cm², 60 cm², 70 cm², 80 cm^{2 2} , 90 cm, 100 cm² area of the burn, or any value in between. The skilled artisan will readily appreciate that various devices, e.g., the JUVAPEN™ injection device (Juvaplus, SA, Switzerland), etc., suitable for the injection of multiple doses of regenerative cells, can be used in the administration of the regenerative cells according to the embodiments disclosed herein. In some embodiments, the regenerative cells are formulated for delivery in a single injection, e.g., a single subcutaneous injection.

In some embodiments, the regenerative cells disclosed herein can be administered via one or multiple intravenous injections. For example, in some embodiments, the regenerative cells can be administered via a single intravenous infusion over a period of 1 min, 2 min, 3 min, 4 min, 5 min, 10 min, 30 min, 45 min, 1 h, 2 h, or longer.

In some embodiments, the regenerative cells disclosed herein can be administered by applying the cells to a scaffold as discussed elsewhere herein (e.g., including but not limited to biocompatible synthetic and non-synthetic matrices, such as skin substitutes), and applying the scaffold seeded with the regenerative cells to the burn. In some embodiments, a scaffold (e.g., including but not limited to biocompatible synthetic and non-synthetic matrices, such as skin substitutes) is applied to the burn, and the regenerative cells disclosed herein are applied onto the scaffold.

Other methods of administering the regenerative cells as disclosed herein include, but are not limited to, those described in Gerlach, et al. (2011) Burns 37, e19-e23. In this method, the regenerative cells are placed into a sterile syringe with a fitted nozzle, and sprayed directly through the nozzle into the burn. Using computer-assisted delivery, the gun distributes cells at a uniform velocity throughout the wound. Such a method could also readily be used to apply the compositions comprising regenerative cells to a scaffold as described herein. The skilled artisan will appreciate that other devices suitable for administering the compositions comprising regenerative cells via spraying the compositions can be used in the methods described herein, including, but not limited to, FIBRIJET® biomaterial applicators (Nordson Micromedics, St. Paul, Minn.), EASY SPRAY® applicators (Baxter, Deerfiled, Ill.), SMARTJET® applicators (Harvest Technologies, Plymouth, Mass.), and the like.

In some embodiments, the compositions including the regenerative cells disclosed herein are administered within 5 min, 10 min, 15 min, 20 min, 30 min, 40 min, 50 min, 1 h, 2 h, 3 h, 4 h, 5 h, 6 h, 7 h, 8 h, 9 h, 10 h, 11 h, 12 h, 24 h, 36 h, 48 h, 60 h, 1 week, 2 weeks, or less, following the burn injury. In some embodiments, the regenerative cells are administered serially over a period of time (e.g., wherein the subject can be administered regenerative cells in a single or in a plurality of doses each time). For example, in some embodiments, the regenerative cells described herein can be administered every 12 hours, every day, every 2, 3, 4, 5, 6, 7, 8, 9, 10, 11, 12, 13, 14 days, every month, or more. The frequency of treatment may also vary. The subject can be treated one or more times per day (e.g., once, twice, three, four or more times) or every so-many hours (e.g., about every 2, 4, 6, 8, 12, or 24 hours). The time course of treatment may be of varying duration, for example, for two, three, four, five, six, seven, eight, nine, ten or more days. For example, the treatment can be twice a day for three days, twice a day for seven days, twice a day for ten days. While our expectation is that the treatment will continue as the patient's tissues go through a healing and/or remodeling process, treatment cycles can be repeated at intervals. For example treatment can be repeated weekly, bimonthly or monthly, and the periods of treatment can be separated by periods in which no treatment is given. The treatment can be a single treatment or can last as long as the life span of the subject (e.g., many years).

In some embodiments, the methods disclosed herein include debriding the burned area, prior to the administration of the compositions disclosed herein. For example, in some embodiments, the methods include a step of removing some, or all, necrotic tissue present as a result of the burn, prior to administration of the compositions disclosed herein. In some embodiments, the burned area is debrided using surgical or mechanical means. In some embodiments, the burned area is debrided using ultrasonic means, e.g., as described in U.S. Pat. No. 80,705,503, and the like. In some embodiments, the burned areas is debrided using pulsing CO₂ lasers are used to debride burn wounds by ablating necrotic tissue, e.g., as described in European Patent No. EP0933096 B1. Various other methods and apparatuses useful for debriding the burned area useful in the embodiments disclosed herein include, but are not limited to, those described in U.S. Patent Application Publication Nos. 20130261534, 20130245386, 20130079800, 20130045196, 20100292689, 20100094265, 20090010869, 20070239078, 20040120989, 20040092920, 20030125783, and the like.

In some embodiments, some, or all of the burned or non-viable tissue, e.g., in the zone of coagulation, is debrided prior to administration of the regenerative cells disclosed herein. In some embodiments, the regenerative cells disclosed herein can be administered both before and following debridement of some or all of the burned or non-viable tissue.

Accordingly, in some embodiments, the regenerative cells as disclosed herein can be administered immediately following debridement of some or all of the burned or non-viable tissue. In some methods, the regenerative cells disclosed herein can be administered 30 min, 40 min, 50 min, 1 h, 2 h, 3 h, 4 h, 5 h, 6 h, 7 h, 8 h, 9 h, 10 h, 11 h, 12 h, 24 h, 36 h, 48 h, 60 h, 1 week, 2 weeks, or longer, following the burn injury.

Accordingly, in some embodiments, the regenerative cells as disclosed herein can be administered immediately prior to debridement of some or all of the burned or non-viable tissue. In some methods, the regenerative cells disclosed herein can be administered 30 min, 40 min, 50 min, 1 h, 2 h, 3 h, 4 h, 5 h, 6 h, 7 h, 8 h, 9 h, 10 h, 11 h, 12 h, 24 h, 36 h, 48 h, 60 h, 1 week, 2 weeks, or longer, before debriding some or all of the burned or non-viable tissue.

As disclosed herein, the regenerative cells can be provided to the subject, or applied directly to the damaged tissue, or in proximity to the damaged tissue, without further processing or following additional procedures to further purify, modify, stimulate, or otherwise change the cells after isolation from the tissue of origin. For example, the cells obtained from a patient may be provided back to said patient without culturing the cells before administration. In several embodiments, the collection and processing of adipose tissue, as well as, administration of the regenerative cells is performed at a patient's bedside. In a preferred embodiment the regenerative cells are extracted from the tissue of the person into whom they are to be implanted, thereby reducing potential complications associated with antigenic and/or immunogenic responses to the transplant. However, the use of cells extracted from or derived from another individual is also contemplated.

### EXAMPLES

The following examples are provided to demonstrate particular situations and settings in which this technology may be applied and are not intended to restrict the scope of the invention and the claims included in this disclosure.

### Example 1

### Viability and Therapeutic Activity of Adipose-Derived Regenerative Cells Isolated from Subjects with Radiation Injury

The experiments described in this example were performed to assess and validate a novel model system useful in studying therapeutics for concomitant radiation injury and thermal burn. The experiments also were performed to assess the safety and efficacy of freshly isolated adipose-derived regenerative cells, delivered subdermally or intravenously, in the treatment of thermal burn in irradiated subjects.

Model System for Radiation Injury and Concomitant Thermal Burn

Pig physiology and skin has been found to be significantly more similar to humans than small mammals. Vardaxis et al., (1997) J Anat 190:601-11. The Gottingen Minipig strain was specifically chosen for this study because of the considerably greater ease of handling and convenience of working with animals that are approximately 10-20 kg at maturity rather than the >100 kg of mature Yorkshire farm swine.

No well-established model evaluating wound healing in the context of non-lethal myelosuppressive total body irradiation exists. Therefore, the experiments described herein describe the development of a novel model system useful for evaluating therapies for concomitant radiation injury and thermal burn. Using the model system developed herein, the ability of adipose-derived regenerative cells to improve healing in animals subjected to full thickness thermal burn injury with concomitant sub-lethal, myelosuppressive total body irradiation in the presence or absence of skin graft was assessed.

To assess healing, two major efficacy histological endpoints (contraction and epithelialization) were evaluated during the course of the study by performing planimetry supplemented with a series of biopsies in the wound bed at specified time points following injury. Because the process of collecting biopsy samples induces a new injury, once-sampled wounds were censored from subsequent analysis. As such, animals were divided into two major groups; Group 1 in which wounds were biopsied at early, medium and late time points with no skin graft and Group 2 in which the wounds were biopsied only after application of a skin graft. The effect of treatment with compositions comprising adipose-derived regenerative cells was evaluated by either comparing data within each Group (1 and 2) or in between the control groups to the individual cell treatment groups.

Besides the injury to skin, thermal burn injury may also be associated with a systemic inflammatory response that can lead to a lethal multi-organ failure. As such, the experiments described herein include treatment by local administration of compositions comprising adipose-derived regenerative cells as well as intravenous infusion of compositions comprising adipose-derived regenerative cells (the latter representing a potential route of administration that may provide a more encompassing systemic effect). Furthermore, it is possible that, in addition to potential effects on systemic inflammatory markers, adipose-derived regenerative cells could also have the potential to positively impact the course of radiation-induced neutropenia and thrombocytopenia. In the studies described herein, the safety and efficacy of a systemic delivery route (intravenous) was evaluated.

Eighteen animals were randomly assigned to one of the groups set forth in Table 1, below:

TABLE 1
EXPERIMENTAL GROUPS AND SUBGROUPS

| | | Treatment | | Number | Wound |
|---|---|---|---|---|---|
| | Sub- | (3 days post | Delivery | of | Biopsies (days |
| Group | group | escharectomy) | Route | Animals | post-injury) |
| 1 | 1a | Vehicle only | Sub-dermal | 3 | 10, 17, 23 and |
| (no | | | | | 33 |
| STSG) | 1b | ADRCs | Sub-dermal | 3 | 10, 17, 23 and |
| | | | | | 33 |
| | 1c | ADRCs | Intravenous | 6 | 10, 17, 23 and |
| | | | | | 33 |
| 2 | 2a | Vehicle only | Sub-dermal | 3 | 17 and 27 |
| (with | 2b | ADRCs | Sub-dermal | 3 | 17 and 27 |
| STSG) | | | | | |

Methods

a. Irradiation A Varian 600c LINAC, was used for animal irradiation. The instrument was set to 100 MU (machine unit) for the dose to be administered and with the dose rate adjusted to 100 MU/min. The LINAC instrument setting needed to deliver a specific radiation dose (1.2Gy) The calculations were conducted for each animal using each animal's individual dimensions, according to manufacturer's instructions.

For the whole body-irradiation, animals were sedated with 1.1 mg/kg acepromazine (IM) and transported to the irradiation room within the LINAC facility. There, the animal was placed into a restraint device (v-shaped foam wedge or sling) with their arms and legs tucked as closely to the body as possible. A bolus tissue-equivalent material (“SuperFlab”) was wrapped around the animal's entire body. The animal was maintained on anesthesia with isoflurane (1-5%) via face mask during the irradiation. Animals received half the target radiation dose on both the right and left lateral surfaces. Diode detectors were placed on each side of the animal and used to measure the sum of entrance and exit radiation doses for animal exposure. Post irradiation, isoflurane was removed, and the animal was returned to its holding cage for recovery until it was returned to the housing room.

b. Thermal Burn. A printed wound template sheet was use to ensure that 6 wound sites in the back of each animal, 3 along each side of the spine, were correctly placed on each animal. Each wound was 3 cm apart and located 4 cm from the spine of the minipig. The template was directly placed onto the pig skin and from cranial to caudal the wound sites were labeled L1, L2, L3 for wounds on the left side and R1, R2, R3 for wounds on the right side.

A customized burn device (see, U.S. Provisional patent application No. 61/979,461) engineered to control the pressure applied on the animal skin during burn creation, was used to induce full thickness burns. At day of injury, animals were cleaned and the dorsal surface of each Minipig was clipped with an electric shaver. The skin was cleaned with chlorhexidine and prepared with DuraPrep (Betadine/alcohol) prior to injury. Preceding the burn induction, the animals were anesthetized by an intramuscular injection of 10-15 mg/kg ketamine with 2 mg/kg xylazine under 3-5% isoflurane via face mask.

For the burn induction, the brass block was heated to approximately 180-200° C., with the temperature being verified by a laser thermometer. For this study, the burn device was calibrated to apply a pressure of 0.4 kg/cm² on the animal's body. Six (6) thermal burns located on the thoracic paravertebral region where the skin surface is flat and large enough to ensure complete contact with the burn device were created using the wound template sheet (3 wounds along each side of the spine). After induction of the burns, the entire wound area was bandaged as described in the approved study protocol to protect the burns from any further self inflicted injuries and from the environment.

A fentanyl patch (25 μg) placed behind the ear on Day 1 for pain management. The patch was changed every other day through Day 11, at which point the study veterinarian determined that the patches could be safely removed from the protocol.

To protect the wounds from outside contamination and infections, a multi-layer dressing was used as follows:
Layer 1 (placed directly on the burn site)—triple antibiotic ointment (Bacitracin, Neomycin, and Polymyxine B Sulfate)
Layer 2—Tegaderm
Layer 3—Ioban2, antimicrobial drapes with an iodophor-impregnated adhesive
Layer 4—the animal was covered with a stocking hose-style shirt
Layer 5—the animal wore a Lomir jacket to hold on all the dressings

c. Isolation of adipose-derived regenerative cells. In order to harvest adipose tissue for processing, animals were anesthetized and a small incision (about 3 inches) was made near the inguinal inlet. Approximately 10-25 g adipose tissue was collected processed using a Cytori® Celution® cell processing device according to manufacturer's instructions (Cytori Therapeutics, Sand Diego, Calif.), in order to obtain a composition comprising adipose-derived regenerative cells. Total cell yield (nucleated cells) and percent viability were determined as described below.

d. Escharectomy. Escharectomies were performed on Day 3, approximately 2 h after adipose tissue collection. Animals were anesthetized by and wound sites were surgically excised, down to the muscle layer. The average total size of the resulting wound was approximately 14 cm².

To protect the wounds from outside contamination and infection, a multi-layer dressing was used. Each dressing was changed every 7 days in order to minimize infection development throughout the course of the study.

The first dressing layer, placed onto the wound site consisted of silver-impregnated soft silicone foam dressing contain (Mepilex-Ag; Molnlycke health Care AB, Goteborg, Sweden). A second dressing layer of Ioban™ (antimicrobial incise drape with an iodophor impregnated adhesive) was applied over the Mepilex to seal off the wound fields. The third layer of coverage consisted of a cotton elastic bandage wrap. Finally, a Lomir jacket was placed to hold all the dressings.

e. Treatment. On Day 3 post-thermal wound induction and post escharectomy, compositions comprising freshly isolated adipose-derived regenerative cells suspended in Lactated Ringer's solution were injected radially and circumferentially into the dermal tissue surrounding the wound (range: 10-16 injections of 0.2 mL/wound perimeter region) as well as directly into the superficial fascia (range: 5-9 injections of 0.2 mL). For local delivery, adipose-derived regenerative cells were locally administered at a dose of 0.23-0.32×10⁶ regenerative cells/cm² into the excised wound, as illustrated in FIG. 2.

For intravenous delivery, freshly isolated adipose-derived regenerative cells suspended in Lactated Ringers were administered intravenously in a total volume of 5 mL. Cell injections were performed through the ear vein at a rate of 1 mL/min with a target viable cell dose of 0.78-3.3×10⁶ regenerative cells/kg body weight.

f. Wound assessment. On Days 3, 10, 17, 23, 27, or 33 post-injury, standardized digital photographs were taken of wounds from various study animals. Wounds were assessed for two parameters: a) contraction (the total area not covered by unwounded skin) and b) epithelialization (the area within the wound showing evidence of neo-epithelialization). FIG. 3 depicts the various areas of the wound as assessed in this study. The green line indicates wound boundary for assessment of contraction; the white line shows boundary of re-epithelialization; the yellow circle indicates the position of a biopsy.

g. Wound biopsy. 6 mm punch biopsies per wound were collected to coincide with dressing changes on Days 10, 17, 23 and 33 post-injury (Group 1) or on Days 17 and 27 post-injury (Subgroups 2a and 2b). If an animal had platelet counts below 50,000/μL, they received only two (2) biopsies per wound site collected for that day. During the course of this study, 7 animals had low platelet counts just prior to a scheduled wound biopsy and therefore only 2 biopsies were collected. On the last time point, the entire wound was collected. Once collected, biopsies were immediately placed in 10% neutral buffered formalin or immediately flash frozen. The absence of a semi-rigid scaffold within the wound led to a higher than anticipated rate of wound contraction. Consequently, at day 17 and later times it was not practical to obtain all four biopsies inside the wounds as planned. Under these circumstances, only two biopsies were collected: one at the center and one at the periphery of the wound. FIG. 4 illustrates the scheduling and processing (IHC or snap-freezing for molecular analysis) for wound biopsy (2 or 4 biopsy collection configuration). Each biopsy was blindly evaluated by 2-3 investigators.

h. Histological analysis. Formalin fixed biopsies were dehydrated, embedded in paraffin, sectioned at 5-μm thickness, and stained with Hematoxylin and Eosin (H&E) by the testing facility. Seven (7) unstained slides were also provided to Cytori personnel for assessment of differential expression of selected markers related to the wound healing process (Masson Trichrome, CD31 and Ki67 staining). After staining, slides were digitally scanned using the Aperio Scan Scope AT2 Turbo and visualized using the ImageScope software.

i. Myelosuppression. Myelosuppression was monitored by regular blood cell counts. Prior to blood collection all animals were sedated with acepromazine (of 0.5-1.1 mg/Kg, IM). Blood was collected via the saphenous vein and placed into vacutainers containing K3EDTA as anticoagulant.

Blood draws were performed 5 days before irradiation injury, and on days 0, 3, 5, 8, 10, 12, 15, 20, 23, 25, 30, and 33 post-irradiation. Blood collected for hematology was using the ADVIA™ 120 Hematology System (Bayer Corporation). Samples that exhibited any evidence of clotting were excluded from the analysis.

Results

a. The model system delivers a consistent, reproducible amount of radiation. All animals in this study received total body irradiation using a bilateral scheme. Based on diode measurements during irradiation, the actual absorbed doses ranged from 1.184 to 1.328Gy, corresponding to a range of 98.7 to 110.7% of target doses (1.2Gy). Detailed actual radiation dose delivered to each animal is shown in Table 2. These data demonstrate that consistent full-body irradiation was delivered.

TABLE 2
INDIVIDUAL RADIATION ABSORBED DOSE.

| | Diode A | | Diode C | | | |
|---|---|---|---|---|---|---|
| | Right | Diode B | Left | Diode D | Average | |
| Animal ID | Shoulder | Right Hip | Shoulder | Left Hip | Dose (cGy) | % of Target |
| 5341010 | 121.6 | 120.3 | 128.8 | 122.9 | 123.4 | 102.80% |
| 5344302 | 130.8 | 130.9 | 135.6 | 133.9 | 132.8 | 110.70% |
| 5348057 | 126.5 | 118.7 | 122.9 | 117.5 | 121.4 | 101.20% |
| 5342130 | 126.8 | 124.9 | 128.4 | 125.4 | 126.4 | 105.30% |
| 5345473 | 127.9 | 126.3 | 129.2 | 127 | 127.6 | 106.30% |
| 5343136 | 122 | 118.5 | 120.1 | 119.5 | 120.0 | 100.00% |
| 5344655 | 135.6 | 127.1 | 134.2 | 128.1 | 131.3 | 109.40% |
| 5341265 | 132.1 | 123.1 | 122.6 | 120.6 | 124.6 | 103.80% |
| 5340536 | 118.7 | 118 | 119.5 | 117.5 | 118.4 | 98.70% |
| 5343063 | 124.5 | 121.3 | 125.7 | 120.3 | 123.0 | 102.50% |
| 5348073 | 124.8 | 120.4 | 122.6 | 119.5 | 121.8 | 101.50% |
| 5346500 | 122.2 | 117.3 | 119.6 | 117.5 | 119.2 | 99.30% |
| 5343195 | 125.2 | 120.1 | 122.4 | 118.7 | 121.6 | 101.30% |
| 5346593 | 124.7 | 119.3 | 122.3 | 120.7 | 121.8 | 101.50% |
| | 126.2 | 118.4 | 121.5 | 119.4 | 121.4 | 101.10% |
| 5343471 | 120.6 | 119.8 | 118.9 | 117.6 | 119.2 | 99.40% |
| 5349592 | 125 | 119 | 120.2 | 118.7 | 120.7 | 100.60% |
| 5346534 | 120.1 | 118.4 | 120.8 | 119.1 | 119.6 | 99.70% |

Doses are expressed in centigray (cGy);
100 cGy = 1 Gy

b. The radiation dose administered in the model system was sufficient to result in myelosuppression and neutropenia. FIGS. 5A-5D illustrate the hematolgocial data (absolute white blood cells, absolute neutrophil, absolute platelets, and absolute lymphocytes) for animals in Group 1, i.e., the “no skin graft” group, including Group 1a (control), Group 1b (local ADRC treatment), and Group 1c (intravenous ADRC treatment). As shown in FIGS. 5A-5D, there were no differences in blood cell counts between the control and either treatment group. Platelet counts in all control animals remained generally stable through Day 5 post-irradiation. Animals exhibited a nadir in platelet count of approximately 100,000/μL or lower between days 10 and 15 after radiation exposure (FIG. 5C). Animals generally exhibited a slow return toward normal platelet levels after 15 days. Animals exhibited a decline in neutrophil counts to below 1,000/μL with a nadir between days 15 and 23 after radiation exposure (FIG. 5B). The average neutrophil counts were generally stable through approximately day 12 post-irradiation. All animals generally exhibited a slow return toward normal neutrophil counts after 25 days. By day 3 post-radiation, a substantial reduction in the number of circulating lymphocytes was observed in all animals. A gradual return towards baseline levels was observed from day 10 onward (FIG. 5D). Similar results were observed in Group 2 animals that received skin grafts, i.e., Group 2a (control) and Group 2b (local regenerative cell treatment). As shown in FIGS. 6A-6D, there were no differences in blood cell counts between the control and treatment group. Further, the animals exhibited the same myleosuppression patterns as the Group 1 animals. Together, these data demonstrate that transient myelosuppression and lymphosuppression was consistently achieved with the selected target radiation dose.

c. Adipose-Derived Regenerative Cells Can Be Obtained From Subjects With Radiation Injury To assess whether exposure to sublethal radiation affects the viability and/or therapeutic efficacy of adipose-derived regenerative cells, the colony forming units (CFU), cell composition, and cell differentiation assays were performed on the animal subjects. Viability and function of adipose-derived regenerative cells isolated from animals subjected to 1.2Gy total body irradiation were assessed.

Adipose-derived regenerative cell yield and viability from animals in subgroups 1b, 1c and 2b were assessed. Overall, an average of 1.5±0.4 adipose-derived regenerative cells were obtained per gram of adipose tissue processed (range: 0.97-2.16×10⁶ cells/g tissue) with average viability of 90.2±4.3% (range: 79.1%-94.4%). This number is within the range of nucleated cells obtained from tissue obtained from non-irradiated subjects (comparative data not shown).

Fluorescence cell sorting analysis for CD45, CD31, CD90 and CD146 was performed to determine the constitution of the cell populations isolated from adipose tissue in the control and irradiated animals. Table 3 shows the expression profiled used to define the different cell sub-populations within the cell populations isolated from adipose tissue.

TABLE 3

Antigen

| Cell subpopulation | CD45 | CD31 | CD90 | CD146 |
|---|---|---|---|---|
| Leukocytes | + | +/− | +/− | +/− |
| Endothelial cells | − | + | +/− | +/− |
| Stromal Cells | − | − | + | +/− |
| Smooth muscle related | − | − | +/− | + |

Exemplary data from the FACs analysis are presented in Table 4, below.

TABLE 4
RELATIVE FREQUENCY OF THE MAJOR CELL
SUBPOPULATIONS IN ADIPOSE-DERIVED CELLS
DERVIED FROM ADIPOSE TISSUE OF ANIMALS
SUBJECT TO RADIATION INJURY

| | | Stromal | Endothelial | Smooth Muscle- |
|---|---|---|---|---|
| Animal ID # | CD45 | Cells | Cells | related Cells |
| Group 1a | 23.2 | 44.6 | 3.3 | 17.6 |
| Group 1a | 22.3 | 24.3 | 10.2 | 29.4 |
| Group 2a | 14.0 | 47.6 | 2.4 | 9.1 |
| Group 2a | 7.0 | 35.4 | 2.2 | 20.3 |
| Average ± | 16.6 ± 7.6 | 38 ± 10.5 | 4.5 ± 3.8 | 19.1 ± 8.4 |
| STD | | | | |

The data above demonstrate that adipose-derived regenerative cells isoalted from irradiated animals were comprised of the same major populations as adipose-derived regenerative cells isolated from non-irradiated farm animals and human specimens as well (comparative data not shown).

The colony-forming unit-fibroblast (CFU-F) assay is a well-established assay used to quantify functional mesenchymal stem cells. See, Hicok, et al. (2011) Methods Mol Biol. 702:87.The CFU-F frequency in the animals sampled was shown to be around 4.62±1.38%. These data demonstrate that adipose-derived regenerative cells isolated from irradiated animals contain an adherent population of cells capable of extensive proliferation in vitro and in a frequency within the same frequency range as reported to human-derived cells (1-6%).To further analyze the functional capability of cells isolated from irradiated versus non-irradiated subjects, adipose-derived regenerative cells isolated from irradiated subjects were assessed in an in-vitro endothelial tube formation assay as previously described. See, e.g., Donovan et al. (2001) Angiogenesis 4:113-121. This assay measures the ability of endothelial cells, given the appropriate time and extracellular matrix support, to migrate and form capillary-like structures (a.k.a. tubes) in vitro. Adipose-derived regenerative cells were plated at a density of 125600 cells/cm² in standard cell culture plates in endothelial cell media (EGM-2, Lonza, Basel, Switzerland). Culture media was changed bi-weekly media changes. After 14 days, cells were allowed to air dry and then fixed using a 50:50 acetone:methanol solution. Fixed cultures were then stained using standard immunohistochemistry techniques and reagents. Exemplary photographs of the immunohistochemical experiments are shown in FIGS. 6A-6B. To verify the endothelial-related origin of the tube like structures observed in the cell culture, the cells were fixed and stained using antibodies against CD146 and CD31. FIGS. 6C and 6D below, the tube-like structures shown in FIGS. 6A and 6B were found to grow on top of a confluent fibroblastic monolayer and expressed both CD146 and CD31, indicative of endothelial cells.

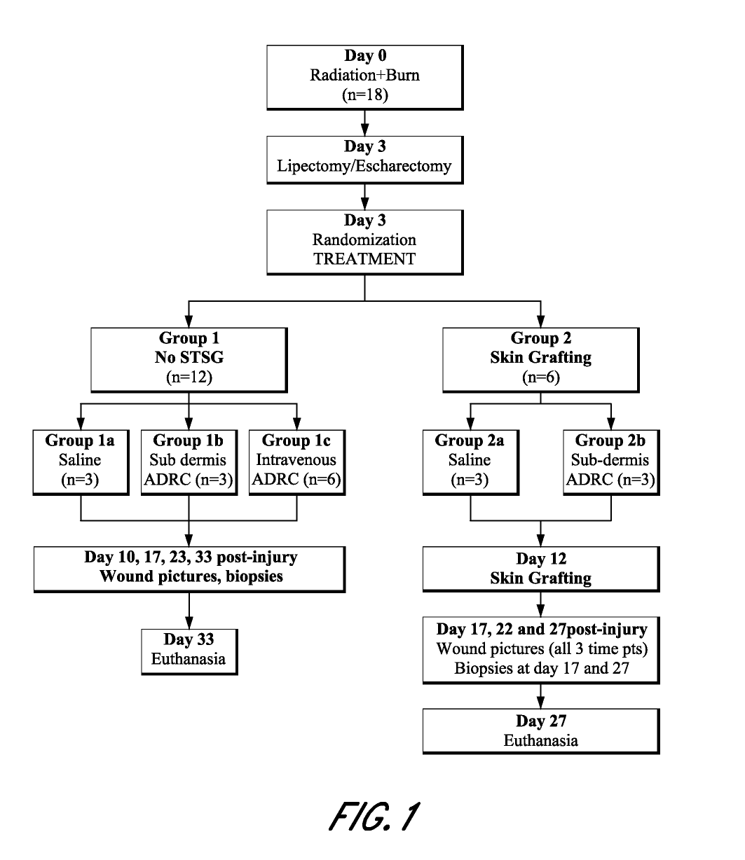

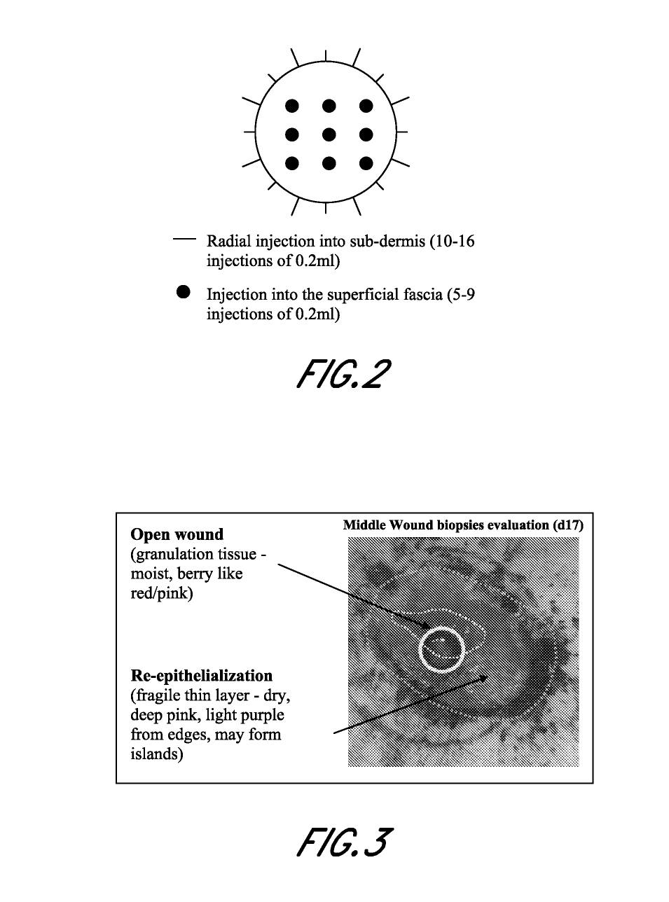

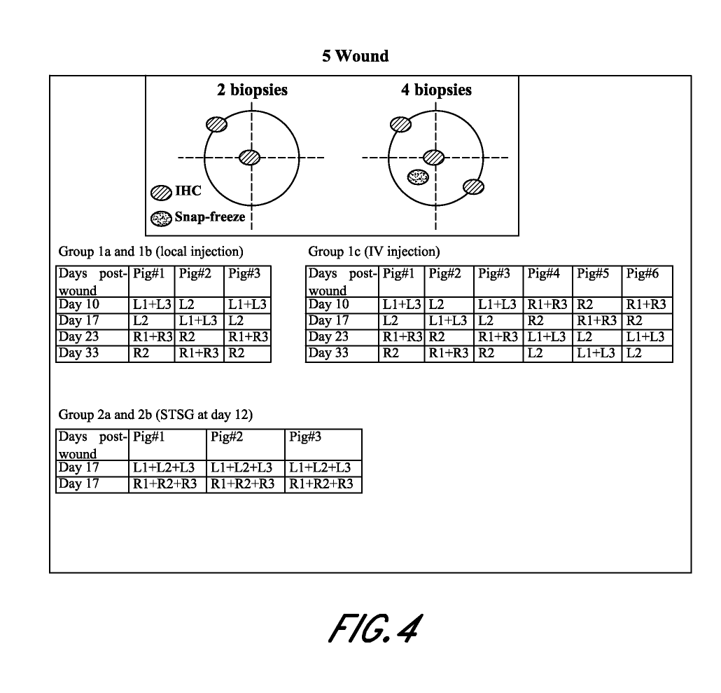

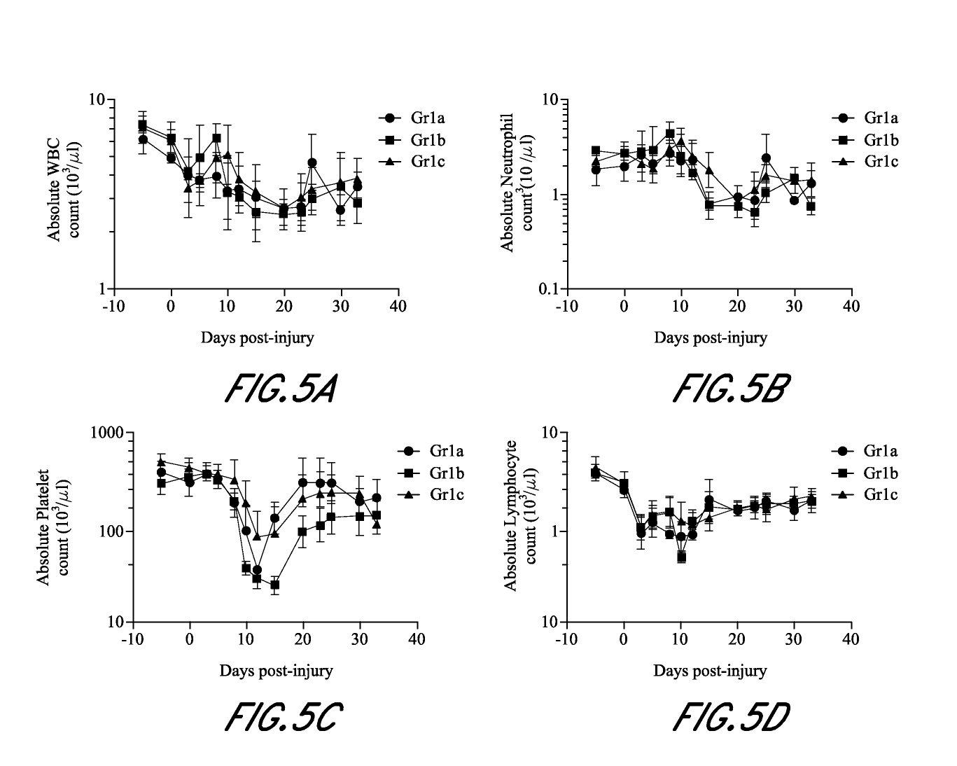

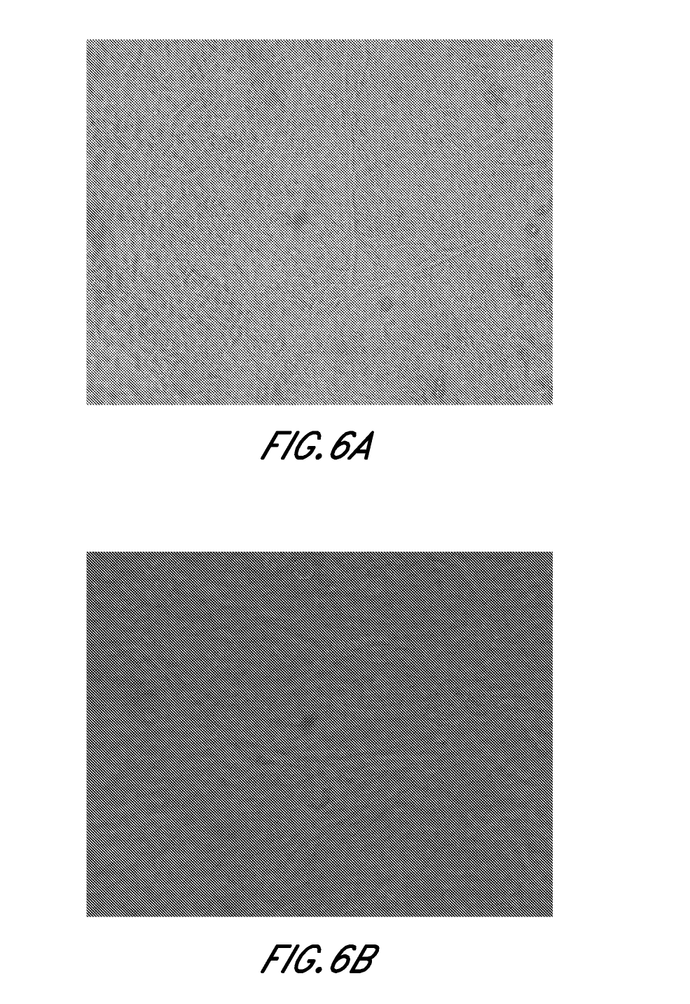

As another test of cell functionality, adipose-derived regenerative cell populations isolated from the animal subjects were assessed for their ability to differentiate into adipocytes using the methods described in [Zuk, et al. (2002) Mol. Biol. Cell 13:4279-4295. FIGS. 7A and 7B show that adipose-derived regenerative cells isolated from irradiated subjects are capable of differentiation into adipocytes.

The foregoing data demonstrate that adipose-derived regenerative can be isolated from adipose tissue of subjects receiving a sub-lethal dose of irradiation in amounts that are similar to non-irradiated subjects. Further, the data demonstrate that the constitution of the regenerative cell populations isolated from adipose tissue of irradiated subjects mirrors that of the regenerative cell populations isolated from adipose tissue of non-irradiated subjects. Finally, the data demonstrate that the adipose-derived regenerative cells isolated from irradiated subjects exhibit the functional capabilities of cells isolated from non-irradiated subjects, e.g., CFU-F, capillary-like formation, and differentiation into adipocytes.

e. Adipose-Derived Regenerative Cells Promote the Healing Process

Wound contraction refers to the movement of the edges of a wound towards the center to close it. This process precedes the maturation stage of healing, and generally occurs between five and 15 days after the original injury is sustained. One concern with wound contraction is the risk of developing a contracture. Ideally, the wound shouldn't tighten too much, or it might create heavy scarring that limits range of motion. This can be a particular concern with full thickness burn wounds over a large extent of the body. These injuries are so large that as they tighten, they may pull against the skin in the region. Patients may need to use physical therapy during healing to retain flexibility and keep the skin supple so it doesn't tighten too much.

To determine the effect of local and intravenous delivery of compositions comprising adipose-derived regenerative cells on wound contraction, wounds were assessed by planimetry at the time of escharectomy (days after injury) and at day 120, 17, 23 and 33 after injury. Wounds were assessed for two parameters: 1) contraction—the total area not covered by unwounded skin; and 2) epithelialization—the area within the wound showing evidence of neo-epithelialization.

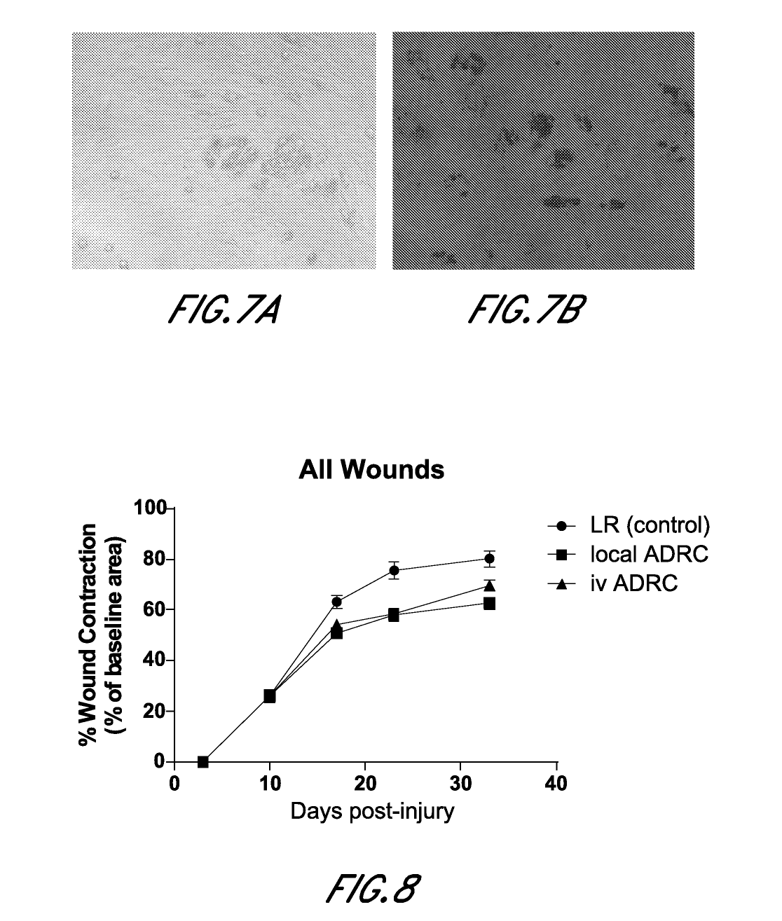

Wound contraction was defined as the change in total wound area as a percentage of the area immediately following wound excision. Three pairs of full-thickness thermal burn wounds were applied to each animal. The wound contraction raw data were modeled with a linear-log relationship (time was log-transformed and then a linear regression was fit to each wound's data) using a hierarchical mixed-effects model. The data for Group 1 are shown in FIG. 8. Importantly, the rate of wound contraction for animals treated with either local injection or intravenous injection of composition comprising adipose-derived regenerative cells was significantly lower than that observed in the control group. For animals in Group 2, no difference in the rate of contraction was observed in the control group and the group treated with compositions comprising adipose-derived regenerative cells (data not shown). It is recognized that scar contracture is the end result of the process of contraction (Goel and Shrivastava, Ind J Plast Surg 2010; 43(Suppl): S63-S71. “Post-burn scars and scar contractures”). Hence, the ability of the regenerative cells to reduce contraction in this study is consistent with utility in the treatment, prevention, and/or reduction in hypertrophic scarring and/or contracture.

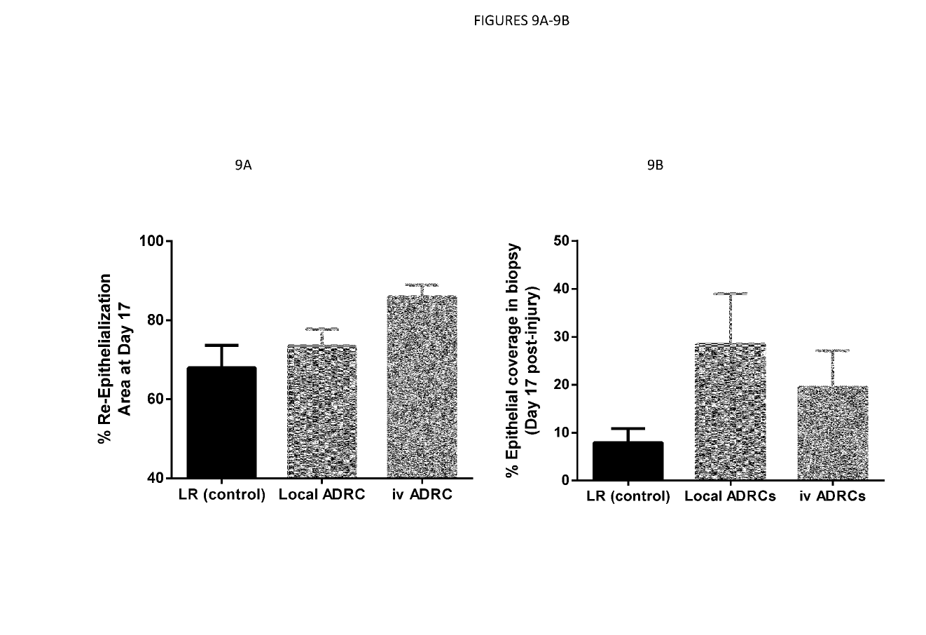

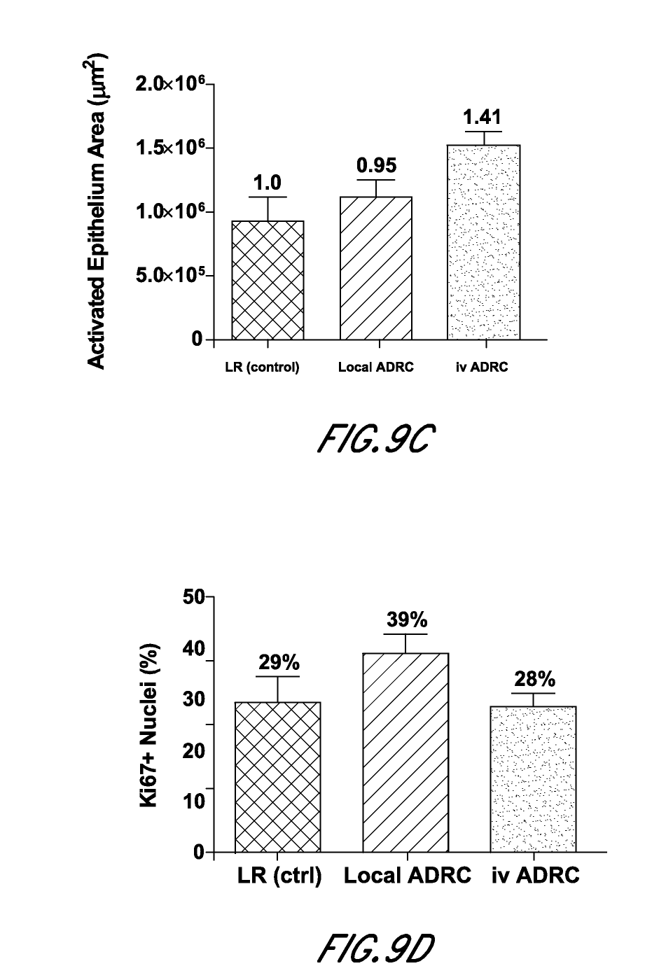

Histological analysis of biopsies collected at the center of the wound showed an increase of epithelial coverage in local and intravenously treated animals in Group 1. See, FIGS. 9A and 9B. Furthermore, histological examination showed that local delivery of compositions comprising adipose-derived regenerative cells enhanced epithelial proliferation at day 7 post-treatment (day 10 post-injury). See, FIGS. 9C and 9D. These data demonstrate that adipose-derived regenerative cells improved wound healing, and further suggest that local and intravenous delivery of regenerative cells may function to improve wound healing and promote epithelial activation by different mechanisms of action, e.g., local delivery of regenerative cells may enhance epithelial proliferation whereas intravenous delivery of regenerative cells may accelerate epithelial migration.

Collagen deposition in the later phage of the wound healing process facilitates greater tensile strength of the wound is a good parameter to evaluate the healing process. Accordingly, collagen deposition in wound biopsies collected at day 33 post-injury was determine using ImageScope™ analysis software using tissue specimens stained with Trichrome Masson dye. The software algorithm uses a deconvolution method to separate different colors, so that quantification of individual stain is possible without cross contamination. The algorithm calculates the percentage of weak (1+), medium (2+), and strong (3+) collagen positive staining. As shown in Table 5, below, local administration of compositions comprising adipose-derived regenerative cells facilitated collagen deposition when compared to control.

TABLE 5

| | Average of | Average of | Average of | |
|---|---|---|---|---|
| | Percent | Percent | Percent | |
| | Strong | Medium | Weak | Average |
| | Positive | Positive | Positive | of Score |
| | (+++) | (++) | (+) | (0-300) |
| Group 1a - | 3.25 ± 2.05 | 9.32 ± 2.91 | 32.77 ± 2.14 | 61.15 ± 13.3 |
| Control | | | | |
| Group 1b - | 8.34 ± 4.63 | 11.89 ± 1.35 | 31.57 ± 3.77 | 80.38 ± 12.7 |
| Local re- | | | | |
| generative | | | | |
| cell delivery | | | | |
| Group 1c - | 5.56 ± 4.09 | 10.50 ± 3.2 | 28.12 ± 4.56 | 65.81 ± 20.13 |
| intravenous | | | | |
| regenerative | | | | |
| cell delivery | | | | |

In short, the data illustrate a statistically significant effect in some of the efficacy parameters for treatment of wounds, e.g., thermal burns, in the context of radiation injury. Specifically, treatment with compositions comprising adipose-derived regenerative cells showed a significant decrease in wound contraction and an increase in wound re-epithelialization compared to animals receiving vehicle alone.

### Example 2

### Adipose-Derived Regenerative Cells can be Obtained from Eschar Tissue

Standard treatment for full thickness burn injury involves excision of non-viable tissue (eschar) in a process referred to as escharectomy. In practice this involves excision down to tissue that exhibits punctate bleeding. Punctate bleeding is clear, visual evidence that the excision has reached a viable tissue bed. The excisional nature of escharectomy thus creates an additional opportunity to obtain adipose tissue from patients with full thickness thermal burns with essentially zero morbidity. For the majority of patients, the escharectomy is performed using a layered, tangential approach carefully preserving the viable tissue underneath. In cases in which this excision exposes underlying adipose tissue, it is possible that adipose tissue can be obtained by simply continuing this excision and excising viable adipose tissue for processing. For patients where bleeding or surgical time are of concern, the eschar is generally excised en bloc down to the fascia in a more aggressive escharectomy process—a process in which the sub dermal adipose tissue is frequently excised along the denatured burned tissue.

The experiments also demonstrate that regenerative cells can be isolated from the adipose obtained from tissue removed during escharectomy (“escharectomized tissue”), and further that these regenerative cell populations have the same characteristics (viability, constitution [e.g., type and frequency of various cell types], and efficacy) as regenerative cell populations isolated from liposuctioned adipose tissue obtained from healthy volunteers. The experiments described below were performed to evaluate in detail the freshly isolated stromal vascular cells obtained by enzymatic processing of adipose tissue obtained from escharectomy, and compare it to the population obtained by processing adipose tissue from non-burned individuals. Adipose-derived regenerative cell yield, viability, CFU-F frequency, cellular composition and differentiation function were analyzed.

Eschar samples from the Burn Center at the University of California, San Diego were transported to Cytori Therapeutics following informed patient consent. Each sample included tissue that in which an en bloc excision surgical approach was used to remove the burned tissue.

A sample of tissue biopsy from the center of the intact eschar was excised and prepared for embedding on paraffin and subsequent histological evaluation prior to dissecting adipose from the specimen. Tissue sections were stained with hematoxylin-eosin and/or with Masson's trichrome following standard histological procedures for histological evaluation using these dyes. Eschar tissue-associated adipose was dissected from the burned skin using scissors and scalpels in a class II biological safety cabinet. Upon isolation, the adipose tissue was weighed and minced into approximately 1-3 mm pieces comparable to those of lipoaspirated adipose using either sterile sharp scissors and/or knifes. The minced tissue specimens were processed to prepare adipose-derived regenerative cells in the Cytori® Celution® cell processing device per manufacturer's instructions Nucleated cell concentration and viability of adipose-derived regenerative cells were assessed using a NUCLEOCOUNTER® cell counting device (Chemometec A/S, Allerod, Denmark), per manufacturer's instructions.

Fluorescence Activated Cell Sorting using fluorescently labeled antibodies directed against, CD31, CD34, CD45, CD90, and CD146 cell membrane proteins was performed to determine the identity of the various cell populations within the heterogeneous population of freshly isolated adipose-derived regenerative cells.

To assess the adipose-derived stem cell frequency in adipose-derived regenerative cell populations isolated from eschar adipose tissue, a CFU-F assay was performed as described above. Briefly, cells were seeded at a concentration of 1,000 cells per well of a standard 6-well culture plate in DME/F12 culture medium supplemented with 10% fetal bovine serum and antibiotic/antimycotic solution. The plates were incubated at 37° C. in 5% CO₂ in a humidified chamber, and the medium was changed once a week. After 12 to 14 days of culture, the cells were fixed and stained using a standard hematologic dye (May-Grunwald) kit. The colonies and clusters were scored using a stereoscope. Six replicate wells were plated for each sample evaluated, and the mean of the middle four counts were used to determine average CFU-F frequency.

To assess the function of the adipose-derived regenerative cells isolated from eschar adipose tissue, the ability of the cells to differentiate into adipocytes was assessed. Adipose-derived regenerative cells (25,000 cells/cm²) were first cultured in standard DME/F12 media supplemented with 10% fetal bovine serum and antibiotic-antimycotic solution at 37° C. in 5% CO₂. At the first media change the non-adherent cells were removed and after the remaining adherent cells had expanded and reached between 70-90% confluence, the standard growth medium was replaced by adipocyte differentiation medium (Zenbio, Research Triangle Park, NC). Cells were maintained in the differentiation medium for 3 days and then the media was replaced with adipocyte maintenance medium. The adipocyte maintenance medium was changed every 3 days until mature adipocytes (lipid-containing cells) were observed (around 7-12 days). After approximately 12 days in culture the cells were fixed in 10% formalin and stained with Oil Red O following standard procedures. Cells that had undergone adipocytic differentiation were evidenced by accumulation of intracellular lipid visualized by red bright stain.

As another functional test of the adipose-derived regenerative cells isolated from eschar adipose, cells were assessed for their ability to differentiate into capillary-like structures in an angiogenesis assay. For the angiogenesis assays, adipose-derived regenerative cells were plated at a concentration of 25,000 cells/cm² in endothelial cell media (EGM-2, Lonza, Basel, Switzerland) and incubated for 7-21 days at 37° C. in 5% CO₂. The medium was changed twice a week, and the cultures examined weekly for tube formation. After 21 days of culture in angiogenic medium, the cells were fixed and the tubular structures were stained with antibodies directed to endothelial proteins (CD31, CD34, CD146, von Wilebrand's Factor) and leukocytic (CD45) markers by immunocytochemistry.

Results:

Histological analysis from eschar tissue showed damaged vessels in the dermis and the subdermal adipose tissue as evidenced by clear presence of vascular hemorrhage in samples collected from the center or periphery of the specimens. See, FIG. 10.

Adipose-derived regenerative cell yield and viability was determined for each eschar sample. The data are presented in Table 6, below.

TABLE 6
YIELD AND VIABILITY OF ADRC IN ESCHAR SAMPLES

| | Surface | | | | | | |
|---|---|---|---|---|---|---|---|
| | Area | Adipose | | | | ADRC Yield | |
| | Specimen | weight | Total | Grams | Process | (cells/gram) | |
| Sample | (cm²) | (g) | adipose | adipose/cm² | Method | (×10⁵) | Viability |
| E1O | 508 | 192.5 | 485.4 | 0.96 | Manual | 2.09 | 92% |
| E1Y | | 157.9 | | | Manual | 1.65 | 90% |
| E2S | 185 | 58 | 126 | 0.68 | Manual | 1.77 | 90% |
| E2D | | 68 | | | Manual | 5.20 | 92% |
| E3 | 1050 | 42.9 | 42.9 | 0.04 | Manual | 2.93 | 79% |
| E5 | 480 | 155.1 | 155.1 | 0.32 | Manual | 0.90 | 91% |
| E6 | 114 | 74.3 | 74.3 | 0.65 | Manual | 1.82 | 92% |
| E7M | 260 | 327.1 | 427.1 | 1.64 | Manual | 3.00 | 94% |
| E7C | | 100 | | | CT-X2 | 1.90 | 86% |
| E8 | 196 | 216.8 | 216.8 | 1.11 | Manual | 2.49 | 93% |
| E9 | 260 | 100 | 100 | 0.38 | Manual | 5.00 | 93% |
| | | | Average | 0.72 ± 0.51 | | 2.61 ± 1.37 | 90 ± 4% |

The average yield and viability of adipose-derived regenerative cells obtained per gram of eschar adipose tissue processed is similar to that for normal donor tissue (comparative data not shown).

Adipose-derived regenerative cell compositions were evaluated by flow cytometry. The major populations in adipose-derived regenerative cells isolated from adipose tissue from normal, healthy donors (stromal, endothelial cells, smooth-muscle related cells, and leukocytes) were defined by a panel of 4 antibodies: CD45, CD31, CD146, and CD34. Endothelial cells were defined as cells expressing both CD34 and CD31, but not CD45; stromal cells were defined as expressing CD34, but not CD31 or CD45; leukocytes were defined as cells expressing the antigen CD45, and smooth muscle-related cells were defined as expressing the antigen CD146, but not CD31 or CD45. Cell subpopulations in the adipose-derived regenerative cell preparations isolated from adipose obtained from eschar tissue are listed in Table 7, below. Importantly, the same major cell populations and frequencies observed in the regenerative cell populations obtained from adipose from normal issue were observed in the adipose-derived regenerative cells isolated from eschar tissue In addition, the same intrapopulation variabity seen in adipose-derived regenerative cells from non injured tissues were also observed in cells isolated from eschar tissue. For instance most but not all, of the CD34+ cells showed to express the marker CD90 and the population CD34+/CD90 accounted for an average of 29.70±15.33% of regenerative cells (ranging from 10.10% to 53.40%). See, FIG. 23.

TABLE 7
MAJOR CELL POPULATIONS IN ADRCS
FROM ESCHAR ADIPOSE TISSUE

| | | | | Smooth |
|---|---|---|---|---|
| | Leukocyte | Endothelial | Stromal | Muscle |
| Sample | (in %) | (in %) | (in %) | (in %) |
| E1Y | 15.70 | 15.70 | 59.20 | 4.20 |
| E1O | 17.35 | 14.30 | 58.00 | 5.20 |
| E2S | 39.60 | 7.60 | 28.00 | 4.25 |
| E2D | 27.00 | 6.35 | 43.75 | 12.90 |
| E3 | 48.30 | 12.60 | 27.15 | 3.45 |
| E5 | 26.80 | 23.55 | 33.30 | 5.30 |
| E6 | 29.75 | 15.40 | 34.55 | 11.45 |
| E7M | 46.90 | 13.10 | 22.70 | 5.10 |
| E7C | 41.20 | 13.20 | 21.60 | 11.80 |
| E8 | 44.70 | 18.70 | 19.60 | 5.20 |
| E9 | 43.00 | 14.00 | 27.90 | 5.80 |
| AVG ± | 34.57 ± 11.73 | 14.05 ± 4.70 | 34.16 ± 13.84 | 6.79 ± 3.46 |
| STD | | | | |

The CFU-F assay performs two functions; it both quantifies the number of putative stem cells within the population and it confirms their proliferative capacity. In this assay, colonies were defined as containing ≧50 cells and cell clusters defined as having more than 4 but less than 50 cells. The average frequency of clusters observed was approximately 1.71% and the average number of colonies was around 1%, in the adipose-derived regenerative cell populations isolated from adipose tissue from escharectomy. The average frequency of colonies from eschar samples was within the range of that reported for adipose-derived regenerative cells isolated from normal donors.

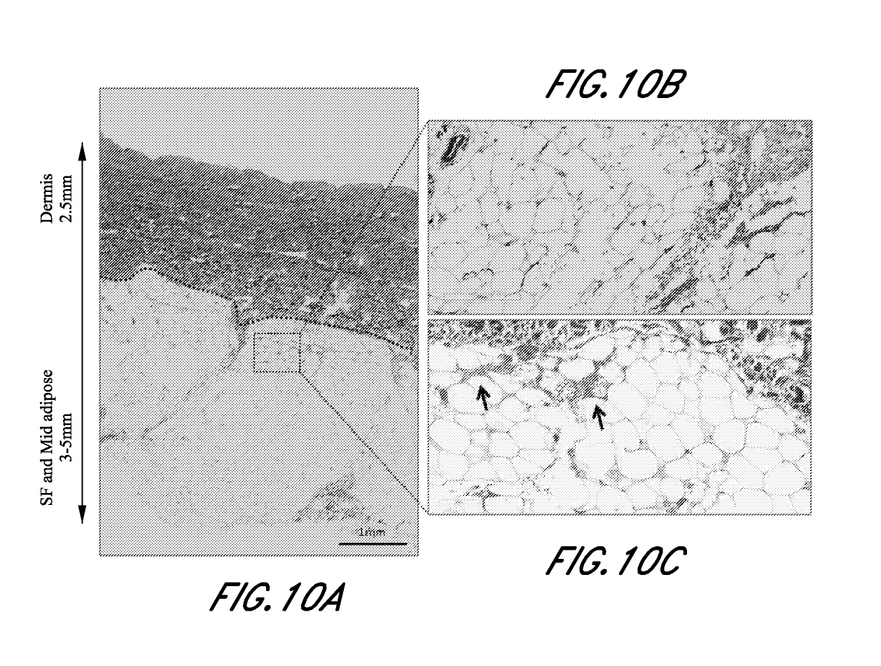

Adipogenic capacity was assessed to analyze the functional capacity of the adipose-derived regenerative cell populations isolated from eschar tissue. FIG. 11 shows Oil Red O staining of an exemplary eschar sample processed and tested as described above. As seen in FIG. 11, an abundant high frequency formation of multilocular adipocytes in was observed in regenerative cell populations isolated from the adipose from eschar samples. This demonstrates that the regenerative cells isolated from adipose from eschar tissue retained the capacity to differentiate into adipocytes.

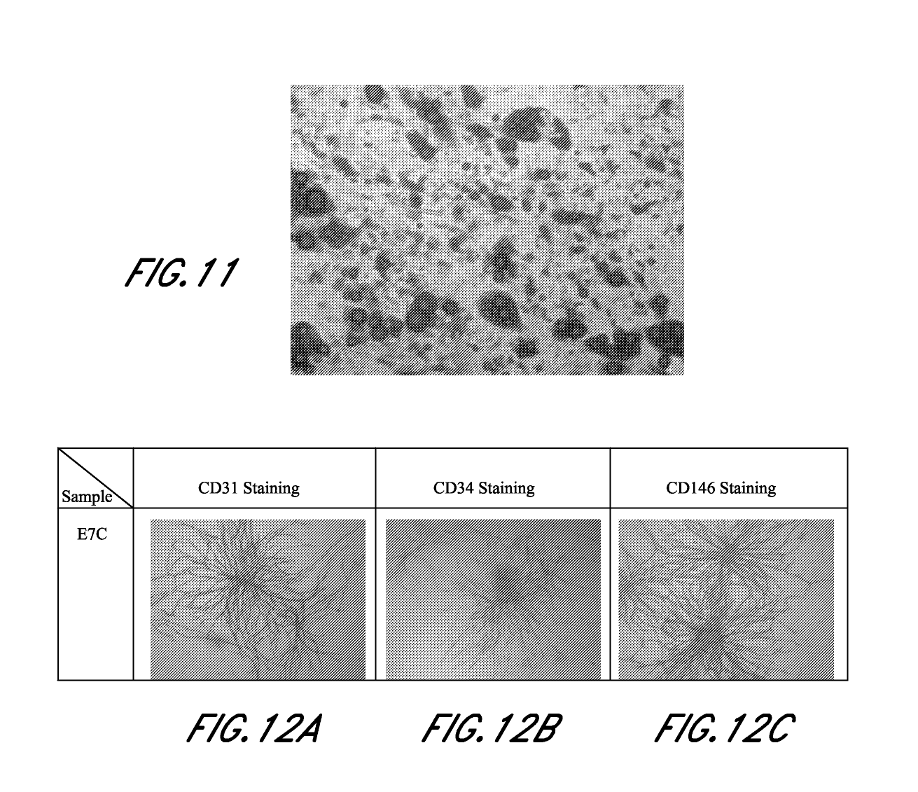

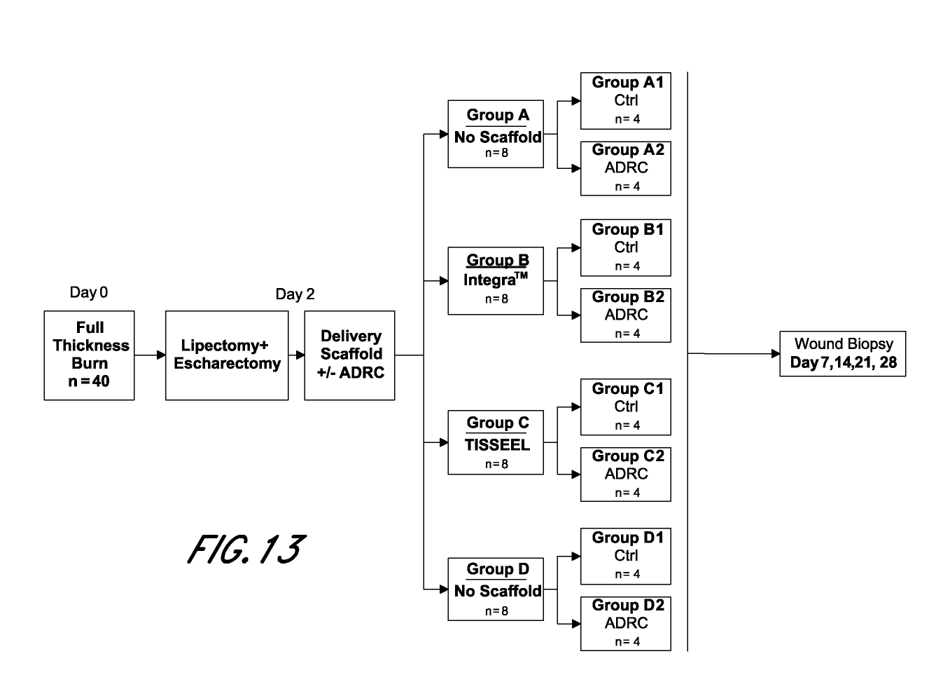

Angiogenic capacity was also assessed as a measurement of functional capacity of the adipose-derived regenerative cell populations isolated from eschar tissue. Donovan et al. have described an assay for angiogenesis in which endothelial cells growing on a feeder layer of fibroblasts-like cells develop a complex network of CD31positive tubes reminiscent of a nascent capillary bed. Previously, Cytori has found that plating normal donor tissue-derived adipose-derived regenerative cells in similar conditions in the absence of an exogenous feeder layer leads to formation of similar structures. Thus, human adipose-derived regenerative cells from eschar samples were cultured in tissue culture plates without the addition of growth factors or Matrigel to evaluate the angiogenic capacity of adipose-derived regenerative cells obtained from the adipose of eschar tissue. An exemplary data set is shown in FIGS. 12A-12C. These data show that as with regenerative cell populations isolated from adipose tissue from unburned subjects, the regenerative cell populations obtained from adipose derived from eschar tissue contained cells that are able to migrate and form tubular structures in the in vitro angiogenic assay.

The foregoing experiments demonstrate that populations of adipose derived regenerative cells can be obtained from adipose from eschar tissue. The cellular composition and viability of adipose-derived regenerative cells isolated from adipose obtained from eschar is similar to that observed in adipose-derived regenerative cells isolated from healthy (i.e., non-burned) tissue. Finally, the adipose-derived regenerative cells isolated from eschar tissue retained the same functional capacities observed in adipose-derived regenerative cells isolated from healthy tissue. These data demonstrate that regenerative cells can be obtained from subcutaneous adipose tissue of patients with thermal burn injury. In this particular study the tissue was obtained by excisional means. Subcutaneous adipose tissue can also be obtained by aspiration (liposuction) and other means recognized in the art.

### Example 3

### Adipose Derived Regenerative Cells Applied with Scaffold Improves Healing

This example demonstrates the utility of adipose-derived regenerative cells in improving wound healing when applied with a scaffold. INTEGRA® and TISSEEL® scaffolds have been used to facilitate wound healing, e.g., the healing of deep partial thickness and full thickness burns. The experiments described herein evaluate the ability of regenerative cells (specifically adipose-derived regenerative cells), to improve healing parameters of INTEGRA® collagen-based dermal regeneration template wound matrix and TISSEEL® fibrin-based wound sealant in full thickness thermal burns.

Twenty four animals were randomly assigned to one of the groups set forth in Table 1, below:

TABLE 8
EXPERIMENTAL GROUPS AND SUBGROUPS

| Group | Test Article | Control Article |
|---|---|---|
| A, *D | Adipose-Derived Regenerative | Lactated Ringer's Solution |
| | Cells (ADRCs) suspended in | |
| | Lactated Ringer's Solution | |
| B | ADRC loaded onto Integra | Integra Wound Dressing |
| C | ADRCs mixed in TISSEEL | TISSEEL/Fibrin Glue |

The experimental process flow is outlined in FIG. 13. Full thickness burns were induced on each animal, and adipose-derived regenerative cells were collected from each animal as described in Example 2, above, in the sections “thermal burn” and “isolation of adipose-derived regenerative cells.” An average of 1.88×10⁶+0.66×10⁶ ADRCs was obtained per gram of processed adipose tissue (range: 0.95×10⁶-2.4×10⁶ cells/g tissue) from Group A animals that received ADRC injection (n=4). The mean recovered cell viability from Group A ADRC preparations was 90.8±3.0% (range: 87.9%-94.8%) Each wound site was surgically debrided by excising the burn site, along with an approximate 2-mm margin, to a full-thickness depth.

Immediately following escharectomy the wound beds of animals in Groups A and D were treated sub-dermal/intrafascial injection with control article 1 (LR vehicle) or test article 1 (adipose-derived regenerative cells suspended in LR). Control/test article were administered as multiple injections delivered radially and circumferentially into the tissue surrounding the wound (10-16 injections of 0.2 mL each per wound bed) as well as directly into the superficial fascia (5-9 injections of 0.2 mL each per wound bed) in a pattern shown in FIG. 2.

For animals in Group B, within ½ hour of cell isolation, ADRCs were directly loaded onto the Integra matrix at a concentration of approximately 3×10⁶ ADRCs in 500 μl per 10 cm² of INTEGRA® Matrix). The matrix loaded with ADRCs was then placed onto the excised wound bed so that the side loaded with ADRCs was in direct contact with the wound bed.

The INTEGRA® matrix loaded with ADRCs was placed onto the excised wound bed so that the surface loaded with ADRCs was in direct contact with the wound bed. After matrix application, the polyethylene sheet was removed. The INTEGRA® matrix for each wound was then was shaped, securely attached with staples onto the wound. The silicone layer was kept in place in the wound throughout the entire course of the study. Specifically, animals in Group B received an ADRC dose within the range of 0.25×10⁶ cells /cm²±25% (i.e. 0.19×10⁶ cells/cm²-0.31×10⁶ cells/cm²). 20×25 cm Integra sheets were cut in 6 pieces of 10×8.3 cm (83 cm²). Using a 1000 μl pipette, ADRCs were evenly loaded onto the INTEGRA® Matrix (2.5×10⁵±25% ADRC per cm² of INTEGRA®, (i.e. 20.8×10⁶±25% ADRCs in 1 mL per 83 cm² of Integra). Cells were then allowed to soak into and adhere to the matrix for 5-10 minutes prior to application onto the wound site.

For animals in Group C, freshly isolated ADRCs were loaded into TISSEEL/Fibrin Glue at a concentration of 2.5-3×10⁶ ADRCs per 10 cm² wound). All animals received an ADRC dose within the range of 0.25×10⁶ cells/cm²±25% (i.e. 0.19×10⁶ cells/cm²-0.31×10⁶ cells/cm²). Freshly isolated ADRCs loaded into TISSEEL were applied at an average dose of 0.24×10⁶±0.04×10⁶ ADRC per cm² (range: 0.17×10⁶-0.37×10⁶/cm². The mean dose of ADRCs in TISSEEL was 0.24×10⁶±0.04×10⁶ ADRCs per cm² of wound area. Control wounds received an equivalent volume of TISSEEL with no cells added.

Following administration of test and control article to each animal, treated sites were bandaged with the following layers: Mepilex, Ioban, cotton wrapping and a Lomir jacket. Follow up measurements, biopsy collection, and blood collection occurred as the animals recovered over the next four weeks. Bandaging was changed once weekly (without reapplication of ADRCs or delivery scaffolds) and topical or systemic antibiotics were applied as needed.

For biopsy, multiple 6 mm punch biopsies per wound were collected to coincide with dressing changes on Days 7, 14, 21, and at in life study phase termination (Day 28). Once a wound had been used for a biopsy at any specific time point, it was not biopsied for the remainder of the study. Biopsies were taken from the center and the periphery on Days 7, 14 and 21 of each wound as illustrated in FIG. 3. Biopsies were collected and were fixed in 10% neutral buffered formalin.

At necropsy on day 28, four biopsies were collected from the appropriate wound (See Table 6 and FIG. 7). Three biopsies 11, 1 and 5 o'clock were fixed in 10% NBF, and one biopsy (7 o'clock position) was collected and snap-freezing for future study.

For wound contraction and re-eptithelialization measurements, wounds were assessed by planimetry, using the SILHOUETTE CONNECT™ digital imaging software (ARANZ Medical, Christchurch, NZ). Wounds were assessed for two parameters: 1) contraction—the total area not covered by unwounded skin and 2) epithelialization—the area within the wound showing evidence of neo-epithelialization.

For histological analyses, biopsied tissues were fixed in 10% Neutral-Buffered Formalin (NBF), dehydrated, embedded in paraffin, sectioned at 3- to 5-μm thicknesses, and stained with hematoxylin and eosin (H&E) and Masson Trichrome stain. Slides (2 sections per biopsy or terminal sample) were qualitatively evaluated via light microscopy by a board-certified veterinary pathologist for assessment of tissue structure, cellularity, and collagen deposition. Additional histological analyses of H&E, Masson's trichrome, and immunohistochemical staining was performed. Slides (one section per biopsy or terminal sample) were qualitatively evaluated via light microscopy by 2 board-certified veterinary pathologists, by assessment of tissue structure, cellularity, and collagen deposition.

For immunohistochemical analyses, paraffin sections of biopsied tissues were deparaffinized and re-hydrated through alcohol to water. Each section was subjected to an antigen retrieval step using sodium citrate solution (pH6, Vector) prior blocking and antibody incubation.

Collagen deposition in wound biopsies collected at day 28 post-injury was determined using ImageScope analysis software using tissue specimens stained with Trichrome Masson. The software algorithm makes use of a deconvolution method to separate different colors, so that quantification of individual stain is possible without cross contamination. This algorithm calculates the percentage of weak (1+), medium (2+), and strong (3+) collagen positive staining. Thus, a collagen deposition score was calculated by a simple formula involving the positive percentages (Score=1x[% weak]+2x[% Medium]+3x[% Strong]). In each biopsy, annotations were created in order to identify superficial and mid/deep region of the granulation tissue Epithelial thickness was evaluated by measuring the length of the epithelial layer from the stratum basale to the stratum corneum on Hematoxylin and Eosin slides. A total of three-five measurements were performed from the left to the right border of the wound scar tissue.

Results

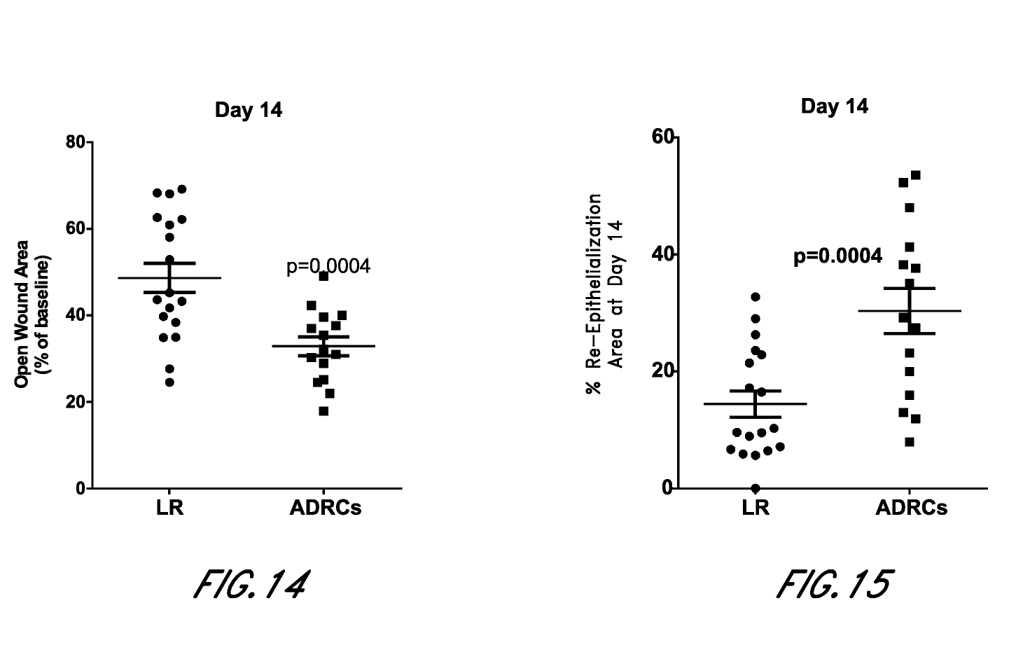

a. ADRCs Improve Wound Closure: Planimetric assessment of Group D control (LR) and test (ADRCs only) treated wounds found that wound closure rate increased by an average of 32% by day 14 post burn induction in animals that received sub-dermal and fascial injections of ADRCs. This is illustrated in FIG. 14 which plots the individual open wound areas of control and test animals. The mean percentage of open wound area in ADRC treated animals is significantly reduced compared to control animals on Day 14 (p=0.0004 by unpaired one-tailed T-test analysis). See, FIG. 14. The increase in wound closure rate observed in Group D ADRC treated wounds was not due to increased rates of contraction since there was no significant difference in mean wound contraction (as measured by planimetry) between the control group (local LR injection) and the ADRC-treated group over the course of the study (data not shown). Planimetric quantification of the wound area in which re-epithelialization had occurred in Group D animals demonstrates an increased rate of epithelialization in the ADRC-treated wounds compared to LR-treated animals. The mean percentage of epithelialization was 30.3%±14.9% (range: 7.9%-53.6%) in ADRC-treated wounds versus 14.4%±9.5% (range: 0%-32.7%) LR-treated wounds respectively. See, FIG. 15. This difference was statistically significant (p=0.0004, by unpaired one tailed t-test).

To determine the kinetics of neovascularization in the wound granulation tissue immunohistochemical analysis of tissue sections was performed on biopsies collected at day 7, 14 and 21 post-injury. Tissue samples from LR- and ADRCs-treated animals were subjected to CD31 (endothelial blood vessel marker) immunostaining to localize neo-capillaries. Each stained slide was digitally scanned and then ImageScope analysis software was applied to quantify microvessel density (number of blood vessels per mm²). In each biopsy, annotations were created in order to identify superficial and deep region of the granulation tissue.

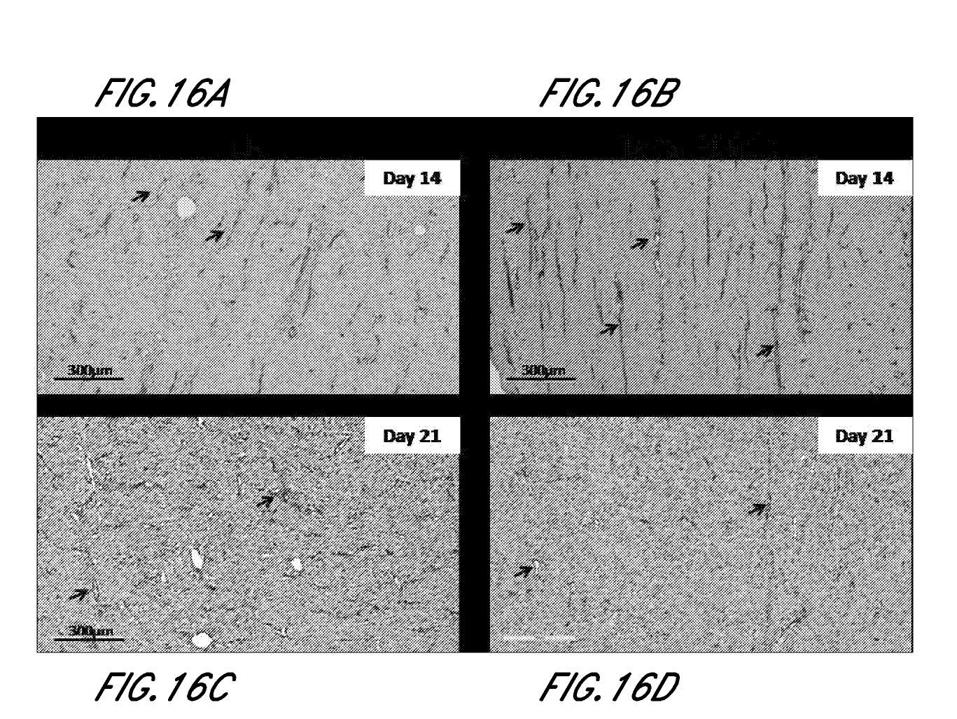

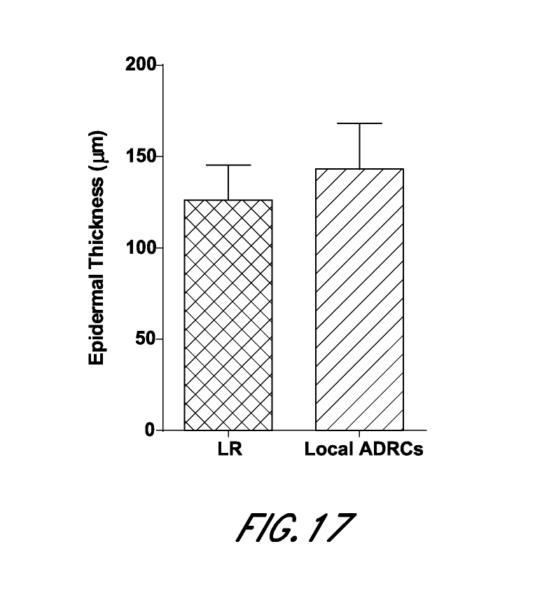

Wound tissues in biopsies collected at day 7 were noticeably heterogeneous. Although CD31 staining was performed, quantification was not feasible due to inconsistent thickness and total area of the granulation tissue in harvested biopsies. Microvessel density (MVD) was significantly increased (1.47-fold) in deep granulation tissue of animals receiving local ADRC treatment compared to LR-treated animals at day 14 (179.6±21.7 versus 121.8±13.3 vessels per mm², respectively; p=0.031). See, FIG. 16A-16D. No significant difference was observed in superficial granulation tissue.

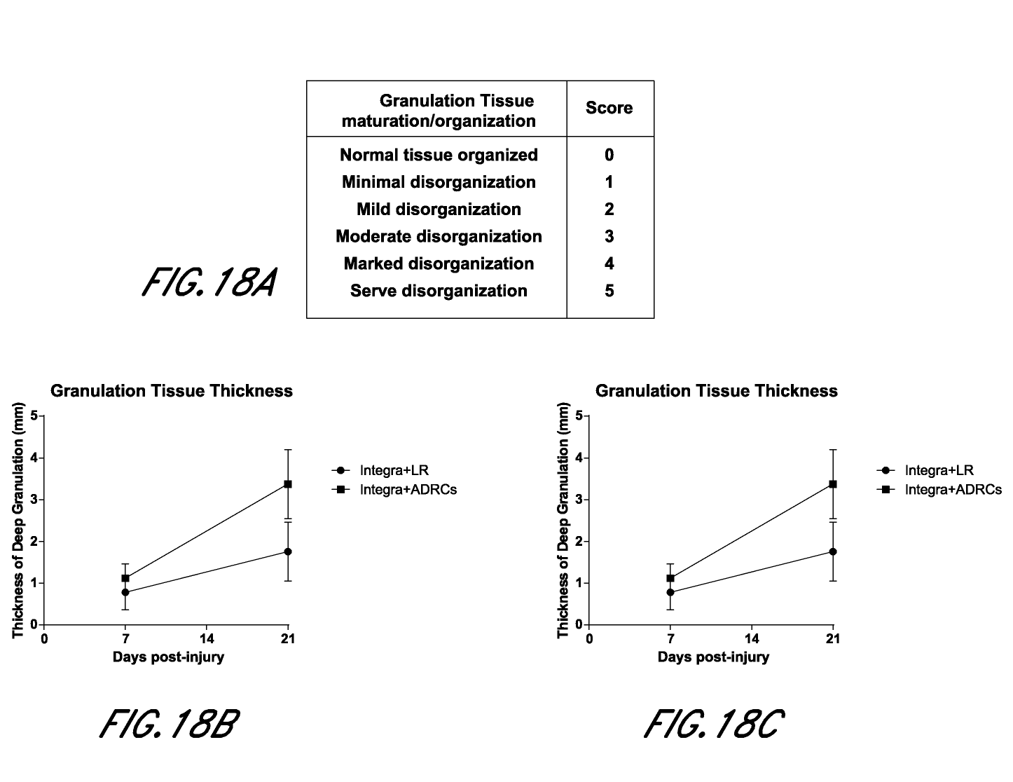

Epithelial thickness was investigated at day 28 in vehicle (Lactated Ringer's, “LR”)- and ADRCs-treated animals in Group D. Mean epithelial thickness at the center of the wound on day 28 was higher in ADRC treated wounds compared to LR treated wounds. See, FIG. 17.

b. Regenerative cells improve INTEGRA® healing Turning to the INTEGRA® dermal matrix, histological scoring by a pathologist blinded to treatment showed accelerated maturation of granulation tissue beneath the Integra when delivered loaded with ADRCs (FIG. 18A, B). As such, the thickness of this granulation tissue was also assessed. Histogenesis starts at the base of the matrix where new blood vessels enter. Layer by layer from base to silicone, a progressive vascularization allows the process to occur at higher levels of the matrix (Gottlied M E, Arimedica, 2005). Interestingly, at day 21, the thickness of the granulation tissue beneath the matrix was increased in animals treated with ADRCs. FIG. 18C. The majority of biopsies collected on Day 14 did not capture a core of INTEGRA® within them, and therefore determination of the granulation tissue thickness was not performed for this time point.

Slides stained with H&E and Masson's Trichrome were evaluated by an outside veterinary pathologist (ANTECH Diagnostics). This individual was blinded as to the treatment applied. By external evaluation, at day 10 post-injury, all biopsies collected from the center of wounds treated with ADRCs, whether by local injection or by intravenous delivery exhibited moderate mixed suppurative and fibrinous inflammation predominating in superficial and mid dermis (score=2.5). Conversely, control animals had moderate mixed suppurative and mononuclear inflammation predominating in superficial and mid dermis (score=2.5). Quantification analysis showed that 100% of the biopsies collected from LR-treated wounds (control) exhibited mononuclear inflammation at day 10. In contrary, 100% of treated wounds (both local and iv injection) exhibited fibrinous. Interestingly, at day 17, 67% of the LR-treated wound showed fibrinous inflammation, whereas treated-wounds still exhibited fibrinous inflammation. These data demonstrate that the adipose-derived regenerative cells modulate the inflammatory response.

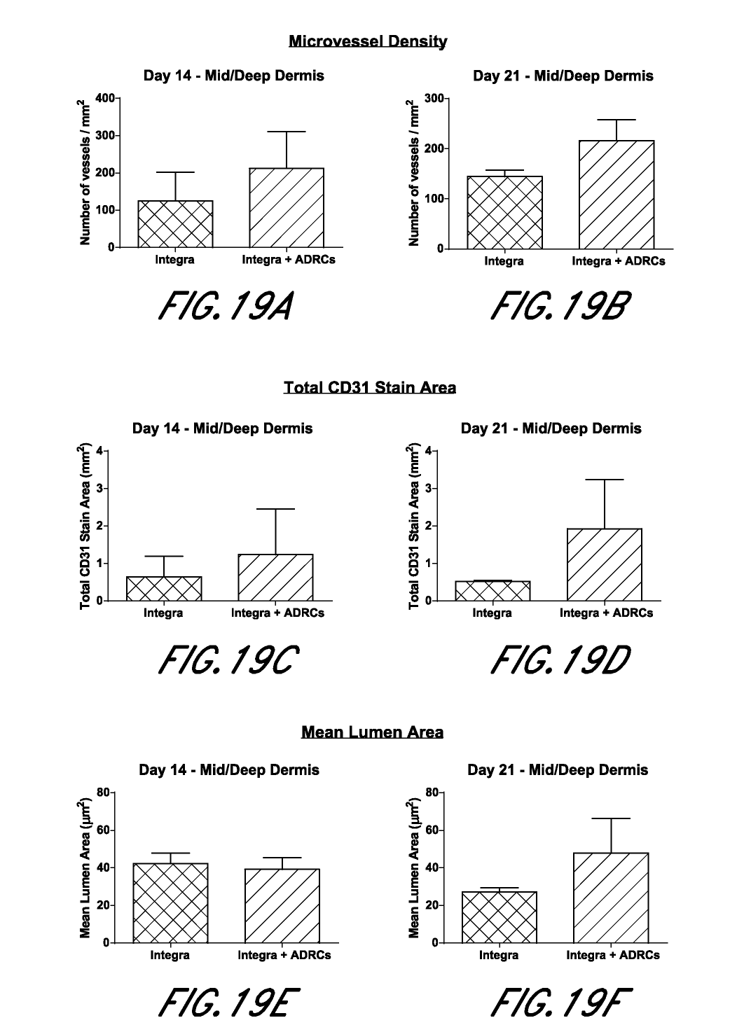

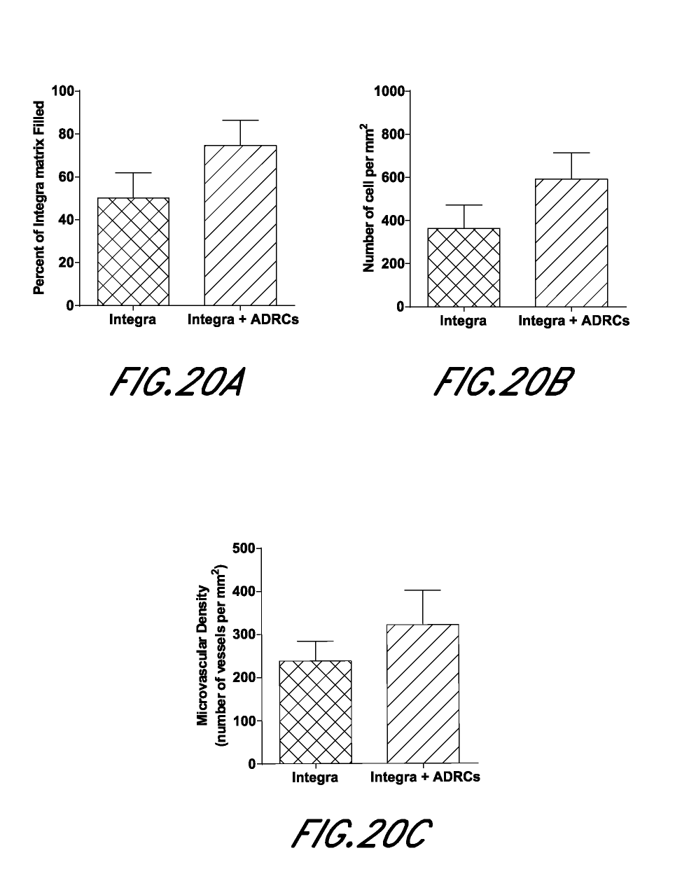

Immunohistological analysis of blood vessel formation was performed to evaluate whether the observed increased thickness of granulation tissue involved accelerated tissue vascularization. To this end, mean vessel density, mean vessel lumen area, and total CD31 stained area were determined to assess the relative maturity of granulation tissue vascularity. Blood vessel density was measured by quantifying the number of CD31 positive vessels within the granulation tissue beneath the Integra sheet on experiment days 14 and 21 post-injury central wound biopsies using ImageScope™ (ARANZ Medical, Christchurch, NZ). Blood vessel density in the granulation tissue below the INTEGRA® matrix was greater in wounds receiving INTEGRA® matrix supplemented with ADRCs-compared to those covered by INTEGRA® matrix alone. This increase approached statistical significance at day 14 (p=0.06) and was statistically significant on day 21 (p=0.024). See, FIGS. 19A-B. The mean vessel lumen area at day 21 in the mid and deep dermis was larger in wounds treated with INTEGRA® matrix loaded with ADRCs than in Integra loaded with LR FIGS. 19E-F. This difference approached statistical significance (p=0.063) The total CD31 stained area in the mid and deep dermis was greater in wounds treated with Integra loaded with ADRCs than in INTEGRA® matrix alone. FIGS. 19C-D. This difference approached statistical significance (p=0.069).

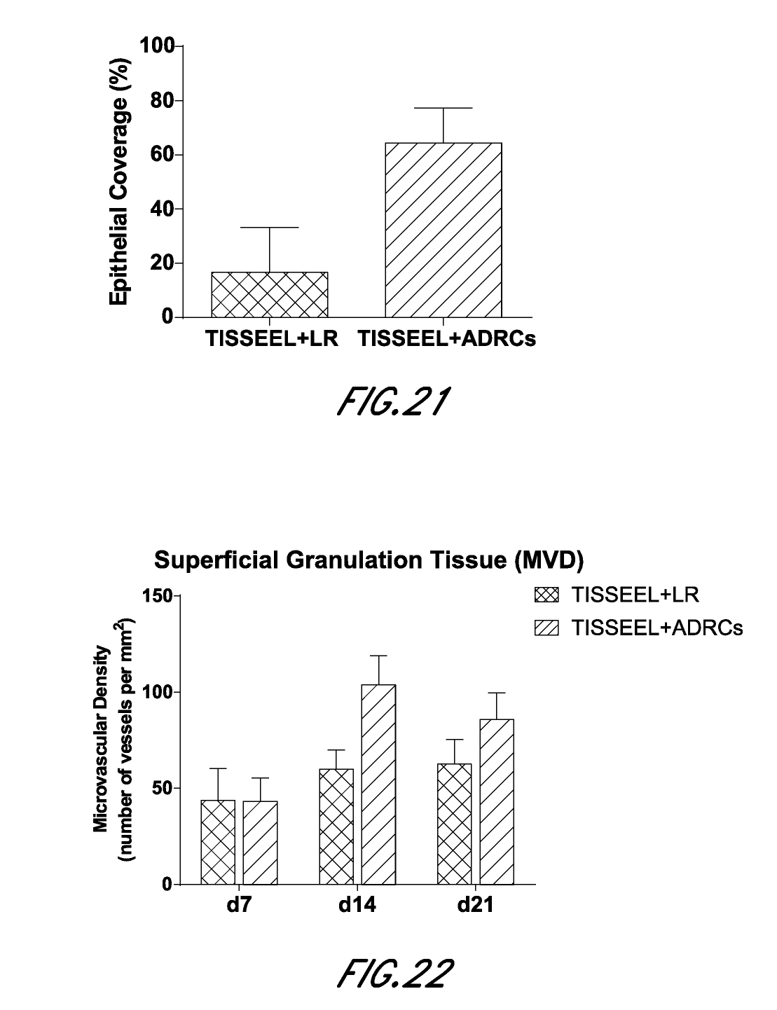

Digital image analysis of biopsies stained with Masson Trichrome, Hematoxylin & Eosin, and CD31 (blood vessel marker) was performed using color deconvolution, nuclear, and vessel density algorithms, respectively (Aperio) to assess the relative contribution of each of the biological processes to matrix filling. Digital quantification revealed that ADRCs loaded onto INTEGRA® matrix increased INTEGRA® matrix cellularity at day 21 post-injury. FIG. 20A-B. INTEGRA® matrix filling and cellularity on study day 21 (n=3-4 animals per group; 6 wounds per treatment). Qualitatively greater cellularity was observed in ADRC loaded Integra compared to INTEGRA® alone. The observed filling effect (FIG. 20A) is statistically significant (p=0.026 by one-tailed T-test), as is the amount of cells within the inter-matrix volume (FIG. 20B) with a higher level of cell nuclei found in ADRC-loaded INTEGRA® (p=0.09 by one-tailed T-testA trend toward increased CD31 positive vessel density was observed in ADRC loaded INTEGRA® matrix relative to INTEGRA® matrix that received no cells (n=3-4 animals per group; 6 wounds treated) at day 21. See, FIG. 20C.

Overall, the data with INTEGRA® matrix scaffold show that compositions comprising adipose-derived regenerative cells improve graft healing. Specifically, regenerative cells improve the vascularization, lumen size, and vessel density of INTEGRA® matrix. Furthermore, the ADRC treated scaffolds exhibit increased cellularity. The data showed accelerated maturation of vessels based upon increased mean lumen size in ADRC-treated wounds, suggesting that ADRCs can favorably modulate vascular stability and blood flow to the new tissue. Indeed, the lumen of a blood vessel is essential for providing blood to the site of injury (Axnick J and Lammert E, Curr Opin. Hematol., 2012). Key dynamic interactions occur between endothelial cells and mural cells (for example, pericytes) to affect vessel remodeling, diameter, and vascular basement membrane matrix assembly, a fundamental process necessary for vessel maturation and stabilization. These processes are critical to control the development of the functional microcirculation. Our findings suggest that ADRCs loaded onto INTEGRA® matrix may orchestrate the complex process of neovascularization by not only promoting angiogenesis but also blood vessel maturation.

c. TISSEEL is an appropriate scaffold for adipose-derived regenerative cell delivery The next set of experiments demonstrate that compositions comprising regenerative cells beneficially enhance healing in the context of other scaffolds, such as TISSEEL®.

Histological assessment of biopsies collected at day 7 reveals the presence of TISSEEL/Fibrin above the growing granulation tissue (data not shown). Furthermore, in ADRCs-treated animals, the presence of migrating cells was observed at the interface granulation tissue/TISSEEL (data not shown).

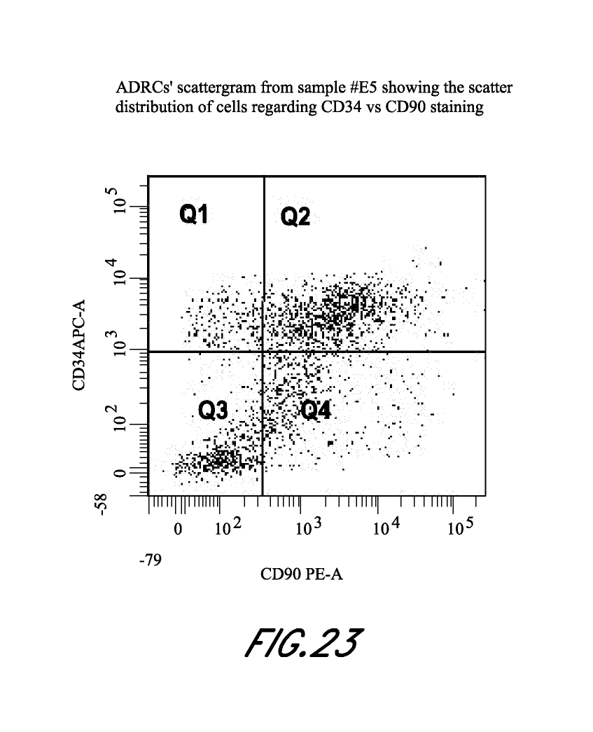

As shown in FIG. 21, supplementation of TISSEEL® with adipose-derived regenerative cells significantly enhanced the epithelial coverage of the recipient site at day 21 post-injury. As shown in FIG. 22, microvascular density was significantly increased (1.72-fold) within the superficial granulation tissue of animals receiving local ADRC treatment compared to LR-treated animals at day 14 (103.7±15.25 versus 60.3±9.9 vessels per mm², respectively; p=0.0325). At day 21, a trend of increased vascularization was observed in ADRCs-treated animals versus LR treatment, (85.8±13.7 versus 62.5±12.9 vessels per mm², respectively) (FIG. 22).

These data demonstrate that fibrin glue scaffolds such as TISSEEL® are appropriate delivery vehicles for administration of adipose-derived regenerative cells to wound sites, e.g., full thickness burn sites.

### Example 4

### Adipose-Derived Regenerative Cells Mitigate Burn Progression in Pigs

This example illustrates the use of adipose-derived regenerative cells as disclosed herein for the prevention or mitigation of burn progression in an animal model.

Animals are randomized to receive treatment with adipose-derived regenerative cells (approximately 1×10⁵-1×10⁷ nucleated cells) or PBS alone (buffer control) administered via subcutaneous injection adjacent to the zone of coagulation approximately 1 hour after injury.

Briefly, four comb burns are created on the back of each animal, using a brass comb preheated in an oven to 100° C., for 5 minutes. This brass comb produces four distinctive burns sites separated by three “interspaces” of unburned skin, which were to undergo progressive injury. (See, e.g., Singer, et al (2007) Acad. Emergency Med. 14:1125-1129. Two full-thickness excisional wounds per pig with the dimensions identical to the comb burns were included as controls.

Full-thickness biopsies from the interspaces 7 days after injury are performed and evaluated for evidence of necrosis after H&E staining. The percentages of interspaces that progress to necrosis are compared with chi-squared (χ2) tests.

At the seventh day, the number of interspaces that processed to full thickness necrosis is significantly lower for the burns treated with adipose-derived regenerative cells compared to the control group, as determined by histologic analysis and macroscopic evaluation at days 2, 5, and 7.

Treatment with compositions comprising adipose-derived regenerative cells (e.g., a concentrated population of adipose-derived cells including stem and precursor cells as disclosed herein), significantly reduces the progression of burn injury in a pig comb burn model.

### Example 5

### Adipose-Derived Regenerative Cells Mitigate Burn Progression in Human

A subject presents with a mid partial-thickness thermal burn over 15% of the subject's total body surface area (TBSA). A unit of adipose tissue is obtained from the subject, and processed according to the methods disclosed in U.S. Pat. No. 7,390,484, whereby a population of adipose-derived regenerative cells is obtained.

Within 24 hours of the burn insult, the subject is administered a composition comprising approximately 1×10⁵-1×10⁷ adipose-derived regenerative cells via intravenous injection.

The mid-partial thickness burn does not progress to a full thickness burn, and the surface area of the zone of coagulation decreases, and does not increase, over time.

### Example 6

### Eschar Tissue-Derived Regenerative Cells Mitigate Burn Progression in Human

A subject presents with a full-thickness thermal burn over 10% of the subject's total body surface area (TBSA). Devitalized (necrotic and/or apoptotic) tissue of the burn is identified and the devitalized tissue is removed via escharectomy.

The escharectomized tissue is mechanically disaggregated by mincing the tissue. The minced tissue is subsequently subjected to enzymatic digestion, to produce a cell suspension. The cell suspension is centrifuged, the resulting cell pellet is resuspended in a physiologic solution (e.g. Lactated Ringer's solution), and passed through a 100 μm filter, thereby providing a concentrated population of regenerative cells. Approximately 1×10⁵-1×10⁷ regenerative cells derived from escharectomized tissue is administered to the subject via subcutaneous injection around the site of escharectomy within 48 hours of the initial burn insult.

The surface area of the full thickness burn and the surface area of the zone of coagulation decreases, and does not increase, over time.

### Example 7

### Adipose-Derived Regenerative Cells Enhance Engraftment and Healing of Autografts

The following example demonstrates that adipose-derived regenerative cells enhance graft take.

Pigs are individually housed under standardized conditions with controlled temperature, humidity and a 12-12 hour day-night light cycle, and are provided free access to water and standard mouse chow.

On day 0, the pigs are randomly divided into a “control” group, and a “treatment” group. Pigs are anesthetized using isoflorane inhalation anesthesia. The dorsum of the pig is shaved, and a circular area with a diameter of 20 mm is outlined on the dorsum at the midline An incision is made along the marking using a scalpel, and the outlined skin is harvested as a full-thickness graft by separating it from the deep dorsal muscular fascia layer. In order to simulate the removal of excess fat from undersurface of the harvested full-thickness skin grafts in clinical conditions, panniculus carnosus layer is removed from the undersurface of the skin graft. For the control group, the grafts are treated with 0.5 ml of a physiological saline solution. For the treatment group, 1×10⁶-8×10⁶ regenerative cells obtained according to the methods disclosed herein are applied to the graft in a 5 ml volume. The grafts are placed back into its donor site by securing the edges with interrupted non-absorbable sutures. The pigs are then caged individually as an additional measure to minimize the trauma to the surgical site.

On day 14 following surgery, the skin grafted areas are macroscopically assessed by using planimetry. Areas with healthy graft tissue and areas that have healed by secondary intention after graft failure are identified. Regions with hair and/or follicles are considered to be healthy graft tissue and areas with a smooth, whitish appearance without hair or follicles are considered to be areas that had healed by secondary intention due to full thickness loss. In order to calculate the size of the healthy regions and the regions healed by secondary intention, these areas are outlined on a transparent paper that was placed on the skin-grafted dorsum. The transparency paper is digitally scanned and the ratio of healthy area to the entire skin graft area was calculated by using computer software (Image Pro Plus, Silver Spring, Md.) for each graft.

On day 14, after the macroscopic assessment of the skin grafts, the pigs are euthanized. The skin grafted area is removed en bloc including the recipient bed and fixed in methanol-Carnoy's solution (methanol:choloroform:glacial acetic acid, 6:3:1). Following this, representative parts composed of healthy graft areas and secondary intention healing are cut out of the main specimens and 4-micron sections were obtained from each specimen for histopathological evaluation.

Standard hematoxylin-eosin staining is performed on representative sections for histopathological evaluation of epithelialization and granulation tissue formation. Each of these parameters was semi-quantitatively evaluated for each representative slide under low power (100×) light microscopy by the pathologist (C.Y.F.) blinded to the source of specimens, in a four-point scale scoring system (0: absent, 1: mild, 2: moderate, 3: abundant). Comparison of data obtained by planimetry for percentage of healthy graft areas as well as the data obtained by semi-quantitative scoring for epithelialization and granulation tissue among control and treated pigs is performed.

The data show that treatment of the skin graft with adipose-derived regenerative cells (e.g., to create a fortified graft), enhances graft take. PIgs with fortified grafts exhibit enhanced granulation tissue formation and enhanced epithelialization scores compared to the control grafts.

### Example 8

### Regenerative Cells Prevent Hypertrophic Scar Formation

A subject presents with a deep partial thickness thermal burn between 5%-30% of the subject's total body surface area (TBSA). Devitalized (necrotic and/or apoptotic) tissue of the burn is identified and the devitalized tissue is removed via escharectomy, in order to crease a recipient site.

The escharectomized tissue is mechanically disaggregated by mincing the tissue. The minced tissue is subsequently subjected to enzymatic digestion, to produce a cell suspension. The cell suspension is centrifuged, the resulting cell pellet is resuspended in a physiologic solution (e.g. Lactated Ringer's solution), and passed through a 100 μm filter, thereby providing a concentrated population of regenerative cells. Approximately 1×10⁵-1×10⁷ regenerative cells derived from escharectomized tissue is administered to the recipient site within 50 days after escharectomy.

The extent and severity of hypertrophic scarring in regions treated with regenerative cells as assessed by the Vancouver Scar Scale (VSS), is lower than that of regions of equivalent injury that were not treated with regenerative cells. For subjects in which the entire region at risk was treated with regenerative cells, the subject does not develop hypertrophic scars, as assessed by the Vancouver Scar Scale (VSS), to the same extent and severity as would be expected in similar patients not treated with regenerative cells.

1-50. (canceled)

51. A method for enhancing incorporation of a skin graft into a recipient wound site in a subject in need thereof, comprising:
providing a skin graft;
administering to the subject a composition comprising adipose-derived regenerative cells; and
applying the skin graft to the recipient wound site thereby enhancing incorporation of the skin graft into the recipient wound site of said subject.

52-55. (canceled)

56. The method of claim 51, wherein the recipient wound site is a burn site.

57. The method of claim 51, wherein the recipient wound site is a non-healing ulcer.

58-59. (canceled)

60. The method of claim 56, wherein the burn is a full thickness burn.

61. The method of claim 51, wherein the subject is human.

62. The method of claim 51, wherein the composition comprises an additive selected from the group consisting of cells, tissue, and tissue fragments.

63. (canceled)

64. The method of claim 51, wherein the skin graft is a split-thickness skin graft.

65-104. (canceled)

105. The method of claim 51, wherein the administering step comprises intravenous administration.

106. A method of accelerating healing of a skin graft at a recipient wound site in a subject in need thereof, comprising:
providing a skin graft;
administering to the subject a composition comprising adipose-derived regenerative cells; and
applying the skin graft to the recipient wound site thereby accelerating healing of the skin graft into the recipient wound site of said subject.

107. The method of claim 106, wherein the recipient wound site is a burn site.

108. The method of claim 106, wherein the recipient wound site is a non-healing ulcer.

109. The method of claim 106, wherein the skin graft is a split-thickness skin graft.

110. The method of claim 51, wherein the skin graft is a meshed autograft.

111. The method of claim 106, wherein the skin graft is a meshed autograft.

112. The method of claim 56, wherein the burn is a thermal burn.

113. The method of claim 107, wherein the burn is a thermal burn.

114. The method of claim 56, wherein the burn is a chemical burn.

115. The method of claim 107, wherein the burn is a chemical burn.

116. The method of claim 106, wherein the administering comprises intravenous administration.

117. The method of claim 56, wherein the burn is a partial thickness burn.

118. The method of claim 107, wherein the burn is a partial thickness burn.
